(19) United States
(12) Patent Application Publication
(10) Pub. No.: US 2024/0268379 A1
(43) Pub. Date: Aug. 15, 2024

(54) BIODEGRADABLE PVA/PVP HYDROGELS, USES AND PREPARATION THEREOF

(71) Applicant: Bar-Ilan University, Ramat Gan (IL)

(72) Inventors: Shlomo MARGEL, Rehovot (IL); Eyal MALKA, Ra’anana (IL); Yulia KABALIN MIZRAHI, Bat-Yam (IL)

(21) Appl. No.: 18/570,313

(22) PCT Filed: Jun. 16, 2022
(86) PCT No.: PCT/IL2022/050651

§ 371 Date: Dec. 14, 2023

Related U.S. Application Data
(60) Provisional application No. 63/211,009, filed on Jun. 16, 2021.

(51) Int. Cl. A01N 25/34; A01N 25/04; A01P 1/00; A01P 21/00

(52) U.S. Cl. CPC A01N 25/34; A01N 25/04; A01P 1/00; A01P 21/00

(57) ABSTRACT

A hydrogel comprising a cross-linked or a non-crosslinked PVA/PVP mixed polymer hydrogel is provided, Further, articles comprising the hydrogel optionally including an active agent incorporated therewithin are also provided.

### CROSS REFERENCE TO RELATED APPLICATIONS

This application claims the benefit of priority under 35 USC § 119(e) of U.S. Provisional Patent Application No. 63/211,009, filed Jun. 16, 2021 titled “BIODEGRADABLE PVA/PVP HYDROGELS, USES AND PREPARATION THEREOF”, the content of which is incorporated herein by reference in its entirety.

### FIELD OF THE INVENTION

The invention relates generally to the field of polymeric hydrogels and articles comprising same.

### BACKGROUND

Polyvinyl pyrrolidone (PVP), also commonly called polyvidone or povidone, is a water-soluble polymer made from the monomer N-vinylpyrrolidone. PVP is also soluble in other polar solvents, e.g., various alcohols, such as methanol and ethanol. When dry it is a light flaky hygroscopic powder, readily absorbing up to 40% of its weight in atmospheric water. In solution, it has excellent wetting properties and readily forms films. This makes it good as a coating or an additive to coatings. PVP like albumin is well known as a physiological carrier for many materials (e.g., hydrogen peroxide, metal ions, essential oils, polymers such as polyvinyl alcohol and polystyrene, iodine, methylene blue, drugs, etc.). PVP also binds to polar molecules exceptionally well, owing to its polarity. PVP is therefore used for many applications such as plasma volume expander, a binder in many pharmaceutical tablets, wetting agent, contact lenses, emulsifier, membranes, a food additive, etc.

Poly(vinyl alcohol) (PVA) is a water-soluble synthetic polymer. It has the idealized formula [CH2CH(OH)]n. It is used in papermaking, textile warp sizing, as a thickener and emulsion stabilizer in polyvinyl acetate (PVAc) adhesive formulations and a variety of coatings. It is colorless (white) and odorless.

PVA is a semi crystalline hydrophilic polymer soluble in water and is the largest volume synthetic resin produced in the world. The excellent chemical resistance, biocompatibility, physical properties, and biodegradability of PVA have led to the development of various commercial products based on this polymer. PVA is a biodegradable polymer with the degradation products being water and carbon dioxide.

Hydrogels are three-dimensional hydrophilic cross-linked polymer networks with characteristically very high swelling ratios in water (they can contain over 90% water). The high-water content and elastic characteristics of hydrogels give them the ability to mimic human tissue better than any other class of synthetic biomaterials. As a result, hydrogels have been considered for a wide range of applications, particularly in drug delivery and tissue engineering.

Currently, there is a great need for development of environmentally friendly polymeric films characterized by low human toxicity and having the capability to carry an agriculturally active agent and control its release over time, such as to combat plant pests or to stimulate plant growth.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

In one aspect, there is a film comprising a coated substrate, wherein the coated substrate comprises a polymeric substrate, wherein at least one surface of the substrate is in contact with a coating layer comprising a hydrogel, wherein the hydrogel comprises PVA and PVP at a w/w PVA:PVP ratio between about 15:1 and about 1:1; a water content of the hydrogel is between 50 and 90% w/w.

In one embodiment, substrate comprises at least partially oxidized surface, comprising a plurality of surface modifications selected from hydroxy, carbonyl, and carboxy, including any combination thereof.

In one embodiment, coating layer is characterized by a dry thickness between 1 μm and 500 μm.

In one embodiment, the coating layer further comprises an active agent, optionally wherein the active agent has an antimicrobial activity, a plant stimulating activity, a pesticidal activity or any combination thereof.

In one embodiment, the active agent comprises hydrogen peroxide (HP), or a precursor thereof, halogen gas precursor, an essential oil, a fertilizer, a pesticide, and a plant hormone, or any combination thereof.

In one embodiment, a weight portion of the active agent within the coating is between 0.01 and 20%.

In one embodiment, the article is characterized by a sustained release of the active agent from the article, wherein the sustained comprises a substantial release of the active agent within a time period between 1 day and 12 months.

In one embodiment, the substrate is stably adhered to the coating layer.

In one embodiment, coating layer is characterized by a visible light transmission (VLT) between 60% and 99%.

In one embodiment, polymeric substrate comprises a polymer selected from polyolefin (e.g. polyethylene (PE), polypropylene (PP), high-density polyethylene (HDPE), low-density polyethylene (LDPE), very low-density polyethylene (VLDPE)), polyethylene terephthalate (PET), PET derivatives, polymethylmethacrylate(PMMA), polystyrene (PS), polyvinyl alcohol (PVA), polycarbonate (PC), silicon rubber polyester, polyvinyl chloride (PVC), polyacetal, cellulose, cellulose derivatives, poly(2-hydroxyethyl methacrylate) (pHEMA), nylon, including any copolymer and any combination thereof.

In one embodiment, the polymeric substrate comprises a polyolefin, the w/w PVA:PVP ratio is between about 4:1 and about 2:1, and wherein the active agent comprises (i) the essential oil and (ii) HP, or a precursor thereof.

In one embodiment, a w/w concentration of the active agent is between about 1 and 10%; and wherein a w/w ratio between (i) and (ii) is between 2:1 and 1:2.

In one embodiment, the article is an antimicrobial article.

In one embodiment, the article is selected from: a film, a ribbon, a package, a wound dressing, a container, and a plant article.

In another aspect, there is an article comprising a hydrogel comprising PVA and PVP at a w/w PVA:PVP ratio between about 15:1 and about 1:1; wherein: a water content of the hydrogel is between 50 and 90% w/w; the hydrogel is cross-linked via a crosslinking agent, the hydrogel further comprises an active agent incorporated within the hydrogel; and a w/w concentration of the active agent within the hydrogel is between 0.1 and 20%.

In one embodiment, the article is manufactured by molding or by casting an aqueous composition comprising the hydrogel.

In one embodiment, a w/w concentration of the cross-linking agent within the hydrogel is between 0.5 and 20%.

In one embodiment, the cross-linking agent is (i) a polyfunctional cross-linking agent comprising a plurality of moieties capable of reacting with PVA, optionally the cross-linking agent comprises a dialdehyde, or a polyaldehyde; or (ii) is a complexation agent capable of forming complexation interaction with PVA.

In one embodiment, the article is an agricultural article, a pharmaceutical article, an antimicrobial article, or an injectable pharmaceutical composition, comprising an effective amount of the active agent.

In another aspect, there is a composition comprising a plurality of beads, each of the plurality of beads comprises a hydrogel comprising PVA and PVP at a w/w PVA:PVP ratio between about 15:1 and about 1:1; wherein: a water content of the hydrogel is between 10 and 90% w/w; the hydrogel further comprises an active agent incorporated within the hydrogel a w/w concentration of the active agent within the hydrogel is between 0.5 and 20%; the hydrogel is cross-linked via a cross-linking agent; and

In one embodiment, a w/w concentration of the cross-linking agent within the hydrogel is between 0.5 and 10%.

In one embodiment, the cross-linking agent is selected from boric acid and a dialdehyde, or any combination thereof, and wherein a w/w PVA:PVP ratio between about 3:1 and about 1:1

In one embodiment, each of the plurality of beads further comprises between 0.1 and 5% w/w of alginate.

In one embodiment, the active agent comprises HP, or a precursor thereof, halogen gas precursor, an essential oil, a fertilizer, a pesticide, and a plant hormone, a viable organism, or any combination thereof.

In one embodiment, an average cross-section of the plurality of beads is between 1 and 50 mm.

In one embodiment, the composition is an agricultural composition comprising an agriculturally effective amount of the active agent, and optionally comprises an agriculturally acceptable carrier, an additive, or both.

In another aspect, there is provided a method comprising applying a pesticide effective amount of the composition of the invention to a plant, to a plant part, or to an area under cultivation, thereby (i) reducing or eradicating a pest, (ii) for preventing or reducing a plant disease associated with the pest, or both (i) and (ii).

In one embodiment, reducing comprises at least 10% reduction, as compared to a control plant or to an area under cultivation, which has not been treated by the composition of the invention.

In one embodiment, the pesticide effective amount comprises between 100 g and 1000 kg of the composition per 1 ha of the area under cultivation.

In another aspect, there is provided a method comprising applying an agricultural effective amount of the composition of the invention to a plant, to a plant part, or to an area under cultivation, thereby (i) controlling plant growth or ripening; (ii) delivering an agricultural agent to the plant.

In one embodiment, applying is performed at any one of the cultivation stages selected from, plating, seeding, harvesting, pre-planting, post planting, pre-seeding, post-seeding, pre-harvesting, post-harvesting, or at the storage of the plant or a plant part, optionally wherein the plant part comprises a fruit, a seed, a leave, a stem, a root, or a combination thereof.

In one embodiment, applying comprises (i) providing the composition in close proximity to the plant, to the plant part; (ii) applying the composition to a soil; or both (i) and (ii).

In addition to the exemplary aspects and embodiments described above, further aspects and embodiments will become apparent by reference to the figures and by study of the following detailed description.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is an image representing the structure of povidone-iodine complex, a common antiseptic.

FIG. 2 is a scheme showing the preparation procedure of PVA/PVP/H₂O₂ hydrogels by a swelling method.

FIG. 3 is a scheme showing the preparation procedure of PVA/PVP/UHP hydrogel.

FIGS. 4A-4B are images of (FIG. 4A) PVA/PVP and (FIG. 4B) PVA/PVP/UHP hydrogel coatings on PE films.

FIG. 5 is an image of PVA/PVP (left) and PVA/PVP/UHP (right) hydrogels nursery tray for plant cultivation.

FIGS. 6A-6B are images of (FIG. 6A) PVA/PVP/UHP hydrogel chips for soil disinfection, and (FIG. 6B) PVA/PVP/UHP hydrogel sheet as a pest control for cuttings.

FIGS. 7A-7B are images of (FIG. 7A) Quantofix® peroxide test stick and color scale (mg/L), and (FIG. 7B) sealed beaker with peroxide test stick and PVA/PVP/UHP hydrogel sample placed on the bottom.

FIGS. 8A-8B are images of (FIG. 8A) inverted 50 mm petri dish with the hydrogel on the top, the virus solution and the peroxide test stick on the bottom and covered with 80 mm petri dish, and (FIG. 8B) 50 mm petri dish with the hydrogel on the bottom with the virus solution and covered with 80 mm petri dish.

FIG. 9 is a line graph demonstrating the effect of thickness of the PVA/PVP hydrogel on the H₂O/H₂O₂ swelling kinetics. Thin layer (0.1 cm, orange), medium layer (0.25 cm, grey) and thick layer (1.2 cm, blue)

FIG. 10 is a line graph showing the release kinetics of H₂O₂ from PVA/PVP/H₂O₂ hydrogel to water. The H₂O₂ percentage (w/w) released from the swollen hydrogen was about 73% within the first 5 min. and 100% (8.6%) within about 15 min. The hydrogel is capable of binding and diffusing the H₂O₂ solution from the surface throughout the 3D matrix of the hydrogels. The relatively slower H₂O₂ kinetics release from the hydrogel is due to the bound H₂O₂ molecules caged within the hydrogel 3D matrix. The matrix bound H₂O₂ molecules need to diffuse to the surface and only then can be released to the aqueous environment.

FIG. 11 is a line graph showing the release kinetics of H₂O₂ from PVA/PVP/UHP hydrogel to water with 0 (blue), 1 (orange), 3 (grey) and 6 (yellow) freeze/thaw cycles.

FIG. 12 is a line graph showing the release kinetics of H₂O₂ vapor from PVA/PVP/UHP hydrogels under different storage temperatures: refrigeration (blue, 3-4° C.), room (grey) and warm (orange, 30° C.) temperatures.

FIG. 13 is a line graph showing % H₂O₂ release kinetics to water of the PVA/PVP/H₂O₂ hydrogel and PVA/PVP/UHP hydrogel.

FIG. 14 is a histogram demonstrating FTIR/ATR spectrum of PVA/PVP hydrogel. The corbonyl stretching, originating from PVP, shifted from 1645 to 1641 cm⁻¹ while the C—O stretching, originating from PVA, shifted from 1084 to 1088 cm⁻¹. These shifts are evidence for the formation of hydrogen bonds forming due to the interaction between hydroxyl groups of PVA and carbonyl groups of PVP.

FIG. 15 is a histogram demonstrating FTIR/ATR spectra of PVA/PVP/H₂O₂ hydrogel versus PVA/PVP hydrogel.

FIG. 16 is a histogram demonstrating FTIR/ATR spectra of PVA/PVP/UHP.

FIG. 17 is a histogram demonstrating FTIR spectrum of urea and hydrogen peroxide released from the PVA/PVP/H₂O₂ hydrogel to the water. The peaks belonging to hydrogen peroxide and urea in the aqueous solution were observed at 2830 and 1160 cm⁻¹ respectively (solid line). H₂O was measured for comparison (dashed line).

FIGS. 18A-18B are light microscope images of (FIG. 18A) PVA/PVP hydrogel, and (18B) PVA/PVP/UHP hydrogel, (magnification ×10).

FIGS. 19A-19D are AFM images of (FIG. 19A) PVA/PVP hydrogel, (FIG. 19B) PVA/PVP/UHP hydrogel, (FIG. 19C) 3D surface morphology image of PVA/PVP hydrogel and (FIG. 19D) 3D surface morphology image of PVA/PVP/UHP.

FIGS. 20A-20H are images showing broccoli seedlings phytotoxic test of PVA/PVP/UHP (left of each image) and PVA/PVP (control, right of each image) hydrogels. Images of the seedlings taken at (FIG. 20A) time zero—planting of the broccoli seedlings, (FIG. 20B) 1 day, (FIG. 20C) 2 days, (FIG. 20D) 3 days, (FIG. 20E) 1 week and (FIG. 20F) 3 weeks. Broccoli seedlings and exposed root system were also imaged after 2 days (FIG. 20G) and 1 week (FIG. 20H).

FIG. 21 is an image of peroxide stick appearance following one day in cooling storage room.

FIGS. 22A-22H are images of (FIGS. 22A-22D) initial seedling growth stage after the cuttings were planted, and (FIGS. 22E-22H) one week after the seedlings were planted. FIGS. 22A and 22E are plants grew from cuttings which were exposed to a PVA/PVP control hydrogel sheet. FIGS. 22B-22D and 22F-22H are plants grown from cuttings which were exposed to PVA/PVP/UHP hydrogel sheet.

FIGS. 23A-23B are images of (FIG. 23A) tobacco seedling, infected with the virus after being inoculated in a PVA/PVP hydrogel and (FIG. 23B) tobacco seedling infected with virus after being inoculated in a PVA/PVP/UHP hydrogel.

FIG. 24 is an image of PVA/PVP hydrogel (left), PVA/PVP/thymol hydrogel (right).

FIG. 25 is a line graph demonstrating FTIR/ATR of thymol in toluene (solid line) against pure toluene (dashed line).

FIG. 26 is a line graph demonstrating FTIR/ATR of PVA/PVP (dashed line) and PVA/PVP/thymol (solid line), hydrogels treated with toluene.

FIG. 27 is an image of the experimental system, including a beaker with plants, insects and hydrogel sheets attached to the wall of the box without any contact with the leaves.

FIGS. 28A-28H are SEM images of exemplary coatings of the invention on a PE film. FIGS. 28A-28D are ×2,000 and FIGS. 28E-28H are ×10,000 magnifications of PVA/PVP unloaded (FIGS. 28A, E,), loaded with: HP (FIGS. 28B, F), thymol (FIGS. 28C, G), thymol and HP (FIGS. 28D, H) hydrogel coatings.

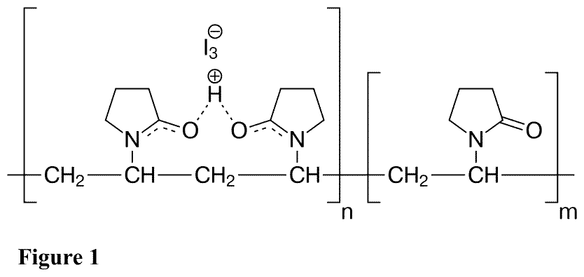

### DETAILED DESCRIPTION

According to one aspect there is provided a composition comprising a polyvinyl alcohol (PVA)-polyvinyl pyrrolidone (PVP) based hydrogel comprising an active agent (e.g., an antimicrobial agent, a plant protecting agent, or both) incorporated therewithin, wherein a weight per weight (w/w) ratio between PVP and PVA within the hydrogel and/or within the composition is between 1:20 and 1:1, between 1:20 and 1:15, between 1:15 and 1:1, between 1:15 and 1:5, between 1:10 and 1:1, between 1:10 and 1:5, between 1:7 and 1:1, between 1:20 and 1:7, between 1:7 and 1:1, between 1:20 and 1:8, between 1:15 and 1:8, between 1:8 and 1:7, between 1:7 and 1:6, between 1:7 and 1:1, between 1:6 and 1:1, including any range or value therebetween.

In some embodiments, the hereindisclosed PVA-PVP hydrogel comprises an active agent bound thereto, wherein bound is via physical interactions (e.g. hydrogen bonding, dipol-dipol interactions, electrostatic interactions, etc.), and wherein a w/w concentration of the active agent within the hydrogel is between 0.01 and 20%, between 0.01 and 10%, between 0.01 and 5%, between 0.01 and 10%, between 0.1 and 20%, between 0.1 and 10%, between 1 and 5%, between 5 and 20%, between 5 and 10%, between 10 and 20%, between 0.01 and 1%, between 10 and 20%, including any range or value therebetween.

In some embodiments, the active agent is adsorbed to the any one of the polymeric constituents of the hydrogel (without being limited to a specific theory, mainly to the PVP). In some embodiments, the active agent is bound to any one of the polymeric constituents of the hydrogel. In some embodiments, the active agent is substantially bound to PVP. In some embodiments, the active agent is further bound to PVA. In some embodiments, the active agent comprises an antimicrobial inorganic compound (e.g. hydrogen peroxide (HP) and/or to a precursor thereof); an essential oil; a pesticide, or any combination thereof. The inventors surprisingly found that PVP has a significantly greater affinity to HP and/or to a precursor thereof, as compared to PVA. Thus, it is postulated that HP and/or precursor thereof incorporated within the hydrogel is primarily bound to PVP.

The hydrogel of the invention, and/or any of the articles/compositions comprising thereof is/are substantially biocompatible or biodegradable, and/or bioerodible. In some embodiments, the term “biodegradable” describes a substance which can decompose under environmental condition(s) into breakdown products. Such environmental conditions include, for example, exposure to open field cultivation conditions such as soil microbiome, irrigation, moisture, rhizosphere, temperature of between 0 and 50° C., UV radiation, hydrolysis (decomposition via hydrolytic cleavage), enzymatic catalysis (enzymatic degradation), and mechanical interactions. As used herein, the terms “biodegradable” and/or “bioerodible” typically refers to materials/articles which are capable of decomposition under these conditions, such that at least 50, at least 70, at least 90 weight percent of the material decomposes within a time period less than 2 years(y), less than 1 y, less than 0.5 y, less than 1 moth, including any range between.

In some embodiments, the term “biodegradable” as used in the context of embodiments of the invention, also encompasses the term “bioerodible”, which describes a material/composition/article which decomposes under environmental conditions into smaller fractions, thus substantially losing its structure and/or mechanical properties. In some embodiments, the term “bioerosion” refers to erosion of the polymeric hydrogel material initiated by water (e.g. by dissolution), microorganisms, enzymes, etc., and resulting in at least partial degradation of the composition/article comprising the bioerodible material.

### Coated Polymer

In one aspect of the invention, there is provided a coated substrate, wherein the coated substrate comprises a polymeric substrate, wherein at least one surface of the substrate is in contact with a coating layer comprising a hydrogel, wherein the hydrogel comprises PVA and PVP at a w/w PVA:PVP ratio between about 15:1 and about 1:1; a water content of the hydrogel is between 10 and 85%, between 50 and 85%, between 10 and 90%, between 10 and 50%, between 20 and 50%, between 10 and 40%, between 20 and 90%, between 40 and 85%, between 60 and 85%, between 10 and 60%, between 10 and 50%, between 60 and 70%, between 70 and 85%, between 50 and 70%, between 60 and 90% w/w, including any range or value therebetween.

In some embodiments, the hydrogel further comprises an active agent incorporated therewithin, wherein the active agent and the concentration thereof within the hydrogel is as described herein. In some embodiments, the active agent is embedded within the hydrogel. In some embodiments, the active agent is enclosed by the hydrogel (e.g. the active agent is located between the hydrogel layers). In some embodiments, the active agent is homogenously distributed within the hydrogel (e.g. as assessed by testing the concentration of the active agent at least 3 different locations on the coated substrate). In some embodiments, the active agent is substantially bound to the polymer, wherein bound is as described herein.

In some embodiments, at least one surface of the polymeric substrate is in contact with the coating layer. In some embodiments, at least one surface of the polymeric substrate is at least partially coated by the coating layer. In some embodiments, between 50 and 99%, between 60 and 99%, between 70 and 99%, between 80 and 99%, between 80 and 90%, between 90 and 99%, between 60 and 99.9%, between 80 and 99.9% of the at least one surface of the polymeric substrate is coated by the coating layer, including any range between. In some embodiments, the terms “in contact with” and “coated by” are used herein interchangeably.

In some embodiments, the polymeric substrate is a layered substrate, comprising at least one polymeric layer. In some embodiments, (i) the polymeric substrate, (ii) the coated substrate of the invention, or both (i) and (ii) is/are water impermeable.

In some embodiments, the polymeric substrate is in a form of a film (e.g. a polymeric film). In some embodiments, the coated substrate of the invention is in a form of a film (e.g. a polymeric film). In some embodiments, the coated substrate of the invention is in a form of a layered film, comprising a first layer in contact with the second layer (e.g. the second layer is on top of the first layer), wherein the first layer is or comprises the polymeric substrate and the second layer is or comprises the coating layer. In some embodiments, any one of the first layer and the second layer is a single layer film, or a multi-layered film comprising a plurality of layers. In some embodiments, any one of the polymeric substrate, the coated substrate, and/or the coating layer, as described herein is/are in a form of a continuous layer, such as a film.

In some embodiments, the term “layer”, refers to a substantially homogeneous substance of substantially uniform-thickness. In some embodiments, each layer has a different physical structure and/or a different chemical composition. In some embodiments, each layer has the same physical structure and/or the same chemical composition. In some embodiments, the term “layer”, refers to a polymeric layer.

In some embodiments, the polymeric substrate is or comprises a polymer characterized by a melting temperature (Tm) between 50 and 300° C., between 50 and 55° C., between 55 and 60° C., between 60 and 70° C., between 70 and 80° C., , between 80 and 90° C., between 90 and 100° C., between 100 and 110° C., between 110 and 120° C., between 120 and 130° C., between 130 and 150° C., between 150 and 200° C., between 200 and 220° C., between 220 and 250° C., between 250 and 270° C., between 270 and 300° C., including any range or value therebetween.

In some embodiments, the polymeric substrate is or comprises a thermoplastic polymer. In some embodiments, the coated substrate of the invention is a thermoplastic polymer.

Non-limiting examples of thermoplastic polymers (e.g. composing the polymeric substrate) include but are not limited to: polyethylene, polypropylene, polyvinyl acetate, polyvinyl chloride, polyvinyl alcohol, polyamide, polyester including any mixture or a copolymer thereof.

Other non-limiting examples of thermoplastic polymers include but are not limited to: polybutadiene, polypropylene-ethylene copolymer, polyethylene, linear low density polyethylene (LLDPE), low density polyethylene (LDPE), medium density polyethylene (MDPE), high density polyethylene (HDPE), isotactic polypropylene, random polypropylene including any mixture or a copolymer thereof.

In some embodiments, the polymeric substrate comprises a polymer selected from polyolefin (e.g. polyethylene (PE), polypropylene (PP), high-density polyethylene (HDPE), low-density polyethylene (LDPE), very low-density polyethylene (VLDPE)), polyethylene terephthalate (PET), PET derivatives, polymethylmethacrylate(PMMA), polystyrene (PS), polyvinyl alcohol (PVA), polycarbonate (PC), silicon rubber polyester, polyvinyl chloride (PVC), polyacetal, cellulose, cellulose derivatives, poly(2-hydroxyethyl methacrylate) (pHEMA), nylon, including any copolymer and any combination thereof.

In some embodiments, the thermoplastic polymer comprises a polyolefin or a co-polymer thereof. In some embodiments, the polyolefin comprises a polyethylene, a polypropylene, polymethylpentene (PMP), polybutene-1 (PB-1); ethylene-octene copolymer, stereo-block polypropylene, propylene butane copolymer, or any combination thereof. In some embodiments, polyolefin is polyethylene.

In some embodiments, the polymeric substrate comprises at least partially oxidized surface. In some embodiments, at least partially oxidized surface is a corona treated surface., wherein the term corona treatment is well-known in the art (exemplary corona treatment is as described in the examples section). In some embodiments, at least one surface of the polymeric substrate (e.g. a surface in contact with the coating layer) is at least partially oxidized surface. In some embodiments, oxidized surface comprises a plurality of surface modifications covalently bound to the outer portion of the polymeric substrate. In some embodiments, the surface modifications are hydrophilic or polar moieties. In some embodiments, the surface modifications comprises any one of: hydroxy, alkoxy, carbonyl (aldehyde and/or ketone), and carboxy (e.g. carboxylic group, and/or, ester group) including any salt and/or any combination thereof.

In some embodiments, the polymeric substrate, and/or the coated substrate is characterized by a thickness between 1 μm and 10 mm, between 10 and 20 μm, between 20 and 40 μm, between 40 and 50 μm, between 50 and 60 μm, between 60 and 70 μm, between 70 and 80 μm, between 80 and 90 μm, between 90 and 100 μm, between 10 and 500 μm, between 100 and 200 μm, between 200 and 500 μm, between 10 and 1000 μm, between 1000 μm and 10 mm, including any range or value therebetween.

In some embodiments, the polymeric substrate is characterized by a thickness between 1 μm and 10 mm, between 10 and 20 μm, between 20 and 40 μm, between 40 and 50 μm, between 50 and 60 μm, between 60 and 70 μm, between 70 and 80 μm, between 80 and 90 μm, between 90 and 100 μm, between 10 and 500 μm, between 100 and 200 μm, between 200 and 500 μm, between 10 and 1000 μm, between 1000 μm and 10 mm, including any range or value therebetween.

In some embodiments, the coating layer is characterized by a thickness between 1 μm and 500 μm, 1 μm and 200 μm, 1 μm and 100 μm, 2 μm and 500 μm, 2 μm and 200 μm, 5 μm and 500 μm, 5 μm and 200 μm, between 10 and 20 μm, between 10 and 100 μm, between 10 and 200 μm, between 20 and 40 μm, between 40 and 50 μm, between 50 and 60 μm, between 60 and 70 μm, between 70 and 80 μm, between 80 and 90 μm, between 90 and 100 μm, between 10 and 500 μm, between 100 and 200 μm, between 200 and 500 μm, including any range or value therebetween. Each possibility represents a separate embodiment of the invention.

In some embodiments, the term “thickness” refers to the dry thickness. As used herein, the term “dry thickness” refers to the thickness of the dried coating layer (e.g. upon substantial evaporation or removal of water). Dried coating layer (or solidified coating layer) refers to a coating layer in a solid state (e.g. non-flowable layer, substantially retaining it's shape and/or dimensions upon tilting thereof). In some embodiments, a water content of the dried coating layer is between10 and 85%, between 50 and 85%, between 10 and 90%, between 10 and 50%, between 20 and 50%, between 10 and 40%, between 20 and 90%, between 40 and 85%, between 60 and 85%, between 10 and 60%, between 10 and 50%, between 60 and 70%, between 70 and 85%, between 50 and 70%, between 60 and 90% w/w, including any range or value therebetween.

In some embodiments, the coating layer is a continuous layer. In some embodiments, the polymeric substrate is in a form of a continuous layer.

In some embodiments, the coating layer is in a form of strips or bands. In some embodiments, the coating layer is in a form of a net. In some embodiments, the coating layer is in a form of intertwined yarns, threads, fibers or strips.

In some embodiments, the coating layer further comprises an active agent, wherein a w/w concentration of the active agent within the coating layer and/or within the coated substrate is between 0.01 and 20%, between 0.01 and 10%, between 0.01 and 5%, between 0.01 and 10%, between 0.1 and 20%, between 0.1 and 10%, between 1 and 5%, between 5 and 20%, between 5 and 10%, between 10 and 20%, between 0.01 and 1%, between 10 and 20%, including any range or value therebetween.

In some embodiments, the active agent is volatile compound. Various volatiles compounds with high vapor pressure under ambient conditions and temperatures between 20 and 30 C are well-known in the art. In some embodiments, the active agent is a viable organism (e.g. a biopesticide for example a Bacillus specie optionally comprising Bacillus thuringiensis; entomopathogenic organism such as entomopathogenic nematode, etc.). In some embodiments, the viable organism is characterized by a pesticidal activity. As used herein, the term “viable” encompasses being capable of: replicating a genome or DNA, cell proliferation or replication, RNA synthesis, protein translation, fermentation or any equivalent energy production process, secretion of active compounds, or any combination thereof.

In some embodiments, the active agent has an antimicrobial activity (e.g. a biocide), a plant stimulating activity (e.g. a plant hormone, a fertilizer, etc.), a pesticidal activity (e.g. a pesticide, a fungicide, a herbicide, an insecticide) or any combination thereof. In some embodiments, the active agent comprises hydrogen peroxide (HP), urea, HP precursor (e.g. urea-hydrogen peroxide adduct, a percarbonate, etc.), a hypochlorite salt, a hypobromite salt, trisodium phosphate-Cl (TSP-Cl), metal ions, ammonium phosphite, methyl orange, cibacron blue, trichloro acetic acid, dichloro acetic acid, mono-chloro acetic acid, trifluoro acetic acid, and/or water insoluble materials, e.g., a plant hormone, an essential oil such as thymol, pheromones such as dodecyl aldehyde or dodecyl alcohol, pesticides such as fluazinam, benzoyl peroxide, or biological pest control agent, an antimicrobial metal ion, or any one of: chlorine (Cl), bromine (Br) and iodine (I) being either in the elemental state or in a form of halide salt or halide ions, or any combination thereof.

In some embodiments, the hydrogen peroxide source is selected from liquid hydrogen peroxide sources (i.e. aqueous solutions of hydrogen peroxide) and solid hydrogen peroxide sources (i.e. solid compounds that upon heating or exposure to water release hydrogen peroxide). Examples of solid hydrogen peroxide sources are, e.g., hydrogen peroxide bound in chemical compounds (e.g. a solid compound of hydrogen peroxide bound in polyvinylpyrrolidone (PVP)) and compounds with the potential of developing hydrogen peroxide, e.g. by reaction with water, such as perborates (e.g. sodium perborate), percarbonates (e.g. sodium percarbonate), peroxyphosphates (e.g. sodium peroxyphosphate), persulfates (e.g. potassium persulfate), peroxymonosulfates, peroxydisulfates, urea peroxide, etc. It should be understood that the hydrogen peroxide source referred to herein may consist of one or more of the species of sources, and possibly also a solid source combined with a liquid hydrogen peroxide source. In some embodiments, the hydrogen peroxide source is selected from sodium peroxide, calcium peroxide, sodium percarbonate, sodium periodate, sodium persulfate, ammonium persulfate, sodium perborate, silver (II) oxide, chlorine dioxide, benzoyl peroxide, a ketone peroxide, a peroxydicarbonate, a peroxyester, a dialkyl peroxide, a hydroperoxide, a peroxyketal or any combination thereof.

In some embodiments, the active agent (e.g. a biocide) comprises a percarboxylic acid (e.g. peracetic acid, peroctanoic acid, perlactic acid, perpropionic acid, percitric acid, and persalicylic acid, performic acid, including any mixture or a derivative thereof), hydrogen peroxide, urea hydrogen peroxide, sodium peroxide, calcium peroxide, silver, silver salt and hydrogen peroxide (HP), sodium percarbonate, sodium periodate, sodium persulfate, ammonium persulfate, perchloric acid, sodium perborate, silver (II) oxide, chlorine dioxide, benzoyl peroxide, a ketone peroxide, a peroxydicarbonate, a peroxyester, a dialkyl peroxide, a hydroperoxide, and a peroxyketal or any combination or salt thereof.

As used herein, the term “essential oil (EO)” refers to a product obtained from a natural raw material of plant origin, by steam distillation, by mechanical processes from the epicarp of citrus fruits, or by dry distillation, after separation of the aqueous phase (if any) by physical processes. EOs are well-known and documented in the art and will be apparent to those skilled in the art. Essential oils suitable according to the present invention, are any essential oil characterized by biocidal activity against a microorganism, such as bacteria, yeast and/or molds. In some embodiments, the essential oil is selected from the group comprising thymol, limonene, cinnamon oil, origanum oil, sage oil tea tree oil, carvacrol oil, or any combination thereof. In some embodiments, the metal ion is any antimicrobial metal ion.

In some embodiments, the metal ion is any biocidal metal ion. As used herein “biocidal metal ion” refers to a metal ion characterized by a biocidal activity. In some embodiments, the metal ion is selected from the group comprising Zn2+, Cu2+or Ag+.

In some embodiments, “biocide” refers to a combination of two or more biocide. In some embodiments, biocide refer to a combination of two biocide. In some embodiments, the coating layer comprises two or more biocides. In some embodiments, the two or more biocides act in synergy. In some embodiments, the two or more biocides comprise (i) an essential oil and (ii) HP, or a precursor thereof. In some embodiments, a w/w ratio between (i) and (ii) is between about 2:1 and about 1:2, between about 2:1 and about 1:1, between about 1:1 and about 1:2, including any range between.

In some embodiments, the active agent described herein is the only active agent within the coated substrate of the invention, within the hydrogel of the invention, and/or within the article comprising thereof.

In some embodiments, any of the composition or article disclosed herein (e.g., polymeric films, beads, and hydrogels) is substantially devoid of an active agent which is not incorporated within the hydrogel, as described hereinabove.

In some embodiments, a w/w concentration of the active agent within the coating layer or within the coated substrate is between about 1 and 10%, between about 1 and 8%, between about 2 and 10%, between about 1 and 5%, between about 5 and 10%, including any range between.

In some embodiments, a w/w concentration of the active agent (e.g. a biocide comprising a single biocide specie) within the coating layer or within the coated substrate is between about 1 and 10%, between about 1 and 8%, between about 2 and 10%, between about 1 and 5%, between about 5 and 10%, including any range between.

In some embodiments, a w/w concentration of the active agent (e.g. a biocide comprising a plurality of biocide species) within the coating layer or within the coated substrate is between about 1 and 10%, between about 1 and 8%, between about 2 and 10%, between about 1 and 5%, between about 5 and 10%, including any range between.

In some embodiments, the active agent comprises (i) the essential oil and (ii) HP, or a precursor thereof and a w/w concentration of the active agent within the coating layer or within the coated substrate is between about 1 and 10%, between about 1 and 8%, between about 2 and 10%, between about 1 and 5%, between about 5 and 10%, including any range between; and wherein a w/w ratio between (i) and (ii) within the coating layer or within the coated substrate is between about 2:1 and about 1:2, between about 2:1 and about 1:1, between about 1:1 and about 1:2, including any range between.

In some embodiments, the coating layer comprises an antimicrobial effective amount (or loading) of the active agent (e.g. biocide). In some embodiments, the antimicrobial effective amount (or loading) of the active agent (e.g. biocide) is between 0.1 μmoles/1cm² and 1000 μmoles/cm², between 0.01 μmoles/1 cm² and 0.1 μmoles/cm², between 0.1 μmoles/1 cm² and 0.5 μmoles/cm², between 0.5 μmoles/1 cm² and 1 μmoles/cm², between 0.1 μmoles/1 cm² and 10 μmoles/cm², between 0.1 μmoles/1cm² and 20 μmoles/cm², between 0.1 μmoles/1 cm² and 40 μmoles/cm², between 1 μmoles/1 cm² and 10 μmoles/cm², between 1 μmoles/1 cm² and 20 μmoles/cm², between 1 μmoles/1 cm² and 40 μmoles/cm², between 10 μmoles/1 cm² and 40 μmoles/cm², between 40 μmoles/1 cm² and 100 μmoles/cm², between 0.1 μmoles/1 cm² and 100 μmoles/cm^{2,} between 0.5 μmoles/1 cm² and 100 μmoles/cm², between 0.5 μmoles/1 cm² and 10 μmoles/cm², between 100 μmoles/1 cm² and 200 μmoles/cm², between 200 μmoles/1 cm² and 500 μmoles/cm², between 500 μmoles/1 cm² and 1000 μmoles/cm², including any range between. Each possibility represents a separate embodiment of the invention.

In some embodiments, the coating layer comprises between 0.1 μmoles/1 cm² and 100 μmoles/1 cm², between 2 μmoles/14 cm² and 100 μmoles/14 cm², between 3 μmoles/1 4cm² and 100 μmoles/14 cm², between 4 μmoles/14 cm² and 100 μmoles/14 cm², between 5 μmoles/14 cm² and 100 μmoles/14 cm², between 2 μmoles/14 cm² and 90 μmoles/14 cm², between 3 μmoles/14 cm² and 90 μmoles/14 cm², between 4 μmoles/14 cm² and 90 μmoles/14 cm², between 5 μmoles/14 cm² and 90 μmoles/14 cm², between 2 μmoles/14 cm² and 50 μmoles/14 cm², between 3 μmoles/14 cm² and 50 μmoles/14 cm², between 4 μmoles/14 cm² and 50 μmoles/14 cm², or between 5 μmoles/14cm² and 50 μmoles/14 cm² of H₂O₂, and/or a precursor thereof, including any range therebetween. Each possibility represents a separate embodiment of the invention.

In some embodiments, the antimicrobial effective amount refers to a minimum amount (or loading) of the biocide sufficient for exhibiting an antimicrobial activity such as the reduction of the microbial load (or CFU of the microbe) on or within the coating layer by at least 2 times, at least 10 times, at least 100 times, at least 1000 times, at least 10.000 times, at least 100.000 times, at least 1000.000 times, including any range between, as compared to a similar coated substrate, wherein the coating layer is devoid of the biocide. Each possibility represents a separate embodiment of the invention.

In some embodiments, the polymeric substrate is a polyolefin; the w/w PVA:PVP ratio within the hydrogel (or within the coating layer) is between about 10:1 and about 2:1, or between about 6:1 and about 2:1, including any range between, and optionally wherein the active agent comprises (i) the essential oil and (ii) HP, or a precursor thereof.

In some embodiments, the term “hydrogel” refers to a solid comprising a supramolecular structures of self-assembled polymeric molecules (i.e. PVA and PVP macromolecules) and water. In some embodiments, the supramolecular structures physically bind water molecules. In some embodiments, the supramolecular structures are in a form of a three-dimensional network of polymeric molecules. In some embodiments, the polymers are homogenously distributed (e.g., dispersed) within the hydrogel, and are substantially devoid of precipitation. In some embodiments, the hydrogel is in a form of a biphasic mixture, or in a form of a polymeric matrix stably bound to the water molecules. In some embodiments, the hydrogel is in a form of a bi-continuous phase (e.g. comprising a layered polymeric matrix and water molecules located in the interphase between the layers), or in a form of a dispersion, such as a colloidal mixture (e.g., polymers distributed within the aqueous phase). In some embodiments, the hydrogel is in a form of amorphous gel, semi crystalline gel, crystalline gel, or hydro colloid gel.

In some embodiments, the average molecular weight (e.g. number average and/or weight average) of PVA is between 10.000 and 200.000, between 15.000 and 200.000, between 20.000 and 100.000, between 50.000 and 200.000, between 50.000 and 150.000 Da, including any range between.

In some embodiments, the average molecular weight (e.g. number average and/or weight average) of PVP is between 10.000 and 100.000, between 15.000 and 80.000, between 20.000 and 100.000, between 20.000 and 50.000, between 10.000 and 50.000 Da, including any range between.

In some embodiments, the hydrogel is substantially amorphous. In some embodiments, the hydrogel is characterized by a crystallinity between 1 and 80%, between 1 and 10%, between 10 and 80%, between 10 and 50%, between 1 and 30%, between 1 and 20%, between 1 and 30%, including any range between. Polymer crystallinity can be determined by various methods, known in the art (e.g. by X-Ray diffraction, such as by XPS).

In some embodiments, the hydrogel or the article comprising thereof is water absorbable, or water swellable. In some embodiments, the hydrogel is configured to absorb up to 300%, up to 250%, up to 200%, up to 100%, up to 70% w/w water including any range between, relative to the initial weight of the hydrogel (e.g. wherein the initial weight refers to the weight of the dried hydrogel), or relative to the dry polymeric content of the hydrogel.

In some embodiments, the polymers within the hydrogel (e.g. PVA) are at least partially cross-linked. In some embodiments, crosslinked is via a covalent cross-link. In some embodiments, crosslinked is via a physical (non-covalent) cross-link. In some embodiments, crosslinked is via a cross-linking agent. In some embodiments, the cross-linking agent is covalently bound to two or more polymeric chains (e.g. PVA chains), also referred to herein as a covalent cross-linking agent. In some embodiments, the cross-linking agent is non-covalently (e.g. via complexation interactions, via electrostatic interaction, etc.) bound to two or more polymeric chains (e.g. PVA chains), also referred to herein as a non-covalent cross-linking agent.

In some embodiments, the term “cross-linking” as used herein refers to the formation of a chemical bond between two chemical moieties or groups. In some embodiments, cross-linking comprises inter cross-linking (e.g. wherein the chemical moieties are distinct polymeric chains). In some embodiments, cross-linking comprises intra cross-linking (e.g. wherein the chemical moieties are within the same polymeric chain).

In some embodiments, the cross-linking agent (e.g. the covalent cross-linking agent) is a polyfunctional cross-linking agent comprising a plurality of moieties capable of reacting with PVA (e.g. with hydroxy groups of PVA). In some embodiments, the plurality of moieties is capable of forming covalent bond with the hydroxy groups of PVA. In some embodiments, the plurality of moieties are selected from carbonyl, active ester (e.g. NHS-ester), sulfonyl chloride, or any combination thereof. In some embodiments, the cross-linking agent (e.g. the covalent cross-linking agent) comprises a dialdehyde, or a polyaldehyde (e.g. polyglutaraldehyde). In some embodiments, the cross-linking agent is selected from boric acid and glutaraldehyde.

In some embodiments, the cross-linking agent (e.g. the non-covalent cross-linking agent) comprises a plurality of moieties capable of forming a non-covalent bond, such as a complexation interaction, electrostatic interaction, etc. In some embodiments, the cross-linking agent (e.g. the non-covalent cross-linking agent) is or comprises boric acid.

In some embodiments, the cross-linked hydrogel comprises at least partially cross-linked PVA. In some embodiments, w/w concentration of the cross-linking agent within the cross-linked hydrogel is between 0.1 and 20%, between 0.1 and 10%, between 0.5 and 20%, between 0.5 and 10%, between 0.1 and 1%, between 0.5 and 3%, between 0.5 and 5%, between 5 and 20%, between 5 and 10%, including any range between.

In some embodiments, w/w ratio between the cross-linking agent and PVA is between 0.1:1 and 1.5:1, between 0.1:1 and 0.5:1, between 0.1:1 and 0.3:1, between 0.1:1 and 0.8:1, between 0.1:1 and 1:1, between 1:1 and 1.5:1, including any range between.

In some embodiments, the hydrogel consists essentially of the polymers (also referred to herein as the polymeric content), water, and optionally comprises the active agent and/or the crosslinking agent.

In some embodiments, the polymeric content of the hydrogel is essentially composed of PVA and PVP. In some embodiments, the w/w ratio of the polymeric content relative to the total weight of the hydrogel or the coating layer is between 10 and 80%, between 10 and 30%, between 10 and 20%, between 10 and 50%, between 10 and 40%, between 10 and 60%, between 10 and 70%, between 5 and 40%, between 5 and 30%, between 5 and 20%, between 5 and 50%, between 5 and 60%, between 5 and 70%, including any range between.

In some embodiments, the polymeric substrate and/or the coated substrate further comprises an additive. In some embodiments, a weight per weight (w/w) ratio of the additive within the polymeric substrate and/or the coated substrate is between 0.1 and 50%, between 0.1 and 0.5%, between 0.5 and 1%, between 1 and 10%, between 1 and 3%, between 3 and 5%, between 5 and 10%, between 10 and 20%, between 20 and 30%, between 30 and 40%, between 40 and 50%, including any range or value therebetween.

In some embodiments, the additive comprises any of: a tackifier, a filler (e.g. a clay particle), a plastomer, an elastomer, a pigment, a dye, an antioxidant (such as a radical scavenger, an antiozonant), a light stabilizer (such as a UV stabilizer), a heat stabilizer, a flame retardant and a biocide or any combination thereof.

Non-limiting examples of additives include but are not limited to 2,4-dihydroxybenzophenone, 2-hydroxy-4-N-(octyl) benzophenone, a derivative of 2-hydroxyphenyl-s-triazine, a hindered amine light stabilizer (HALS), benzotriazole-based UV absorber (such as Tinuvin), or a combination thereof.

In some embodiments, the coated substrate is characterized by a water contact angle on the outer surface of the coating layer (i.e. the surface facing the ambient, as opposed to the inner surface facing the polymeric substrate) between 40° and 100°, between 50° and 100°, between 60° and 100°, between 71° and 100°, between 72° and 100°, between 75° and 100°, between 78° and 100°, between 80° and 100°, between 71° and 98°, between 72° and 98°, between 75° and 98°, between 78° and 98°, between 80° and 98°, between 71° and 90°, between 72° and 90°, between 75° and 90°, between 78° and 90°, or between 80° and 90°, including any range therebetween. Each possibility represents a separate embodiment of the invention.

In some embodiments, the outer surface of the coating layer is characterized by a surface roughness between 1 and 100 nm, between 1 and 1000 nm, between 1 and 50 nm, between 1 and 40 nm, between 1 and 30 nm, between 1 and 20 nm, including any range between. In some embodiments, the surface roughness is predetermined by the chemical composition and/or concentration of the active agent incorporated within the coating layer.

As used herein, the term “ambient” refers to an immediate surroundings. In some embodiments, the ambient refers to the surrounding atmosphere. In some embodiments, the ambient refers to a liquid or a solution. In some embodiments, the ambient refers to the area under cultivation, storage facility, storage container, and/or immediate surrounding of the edible matter (e.g. plant or plant part, such as fruit).

In some embodiments, the coated substrate of the invention is a molded or a casted film or article. In some embodiments, the coating layer is applied on the polymeric substrate by a method selected from coating, spreading, casting, and molding, or any combination thereof.

In some embodiments, the coated substrate of the invention is in a form of a chip, a flock, a sheet, a film article, a packaging article, an agricultural article, a plant-protecting article, or any combination thereof. In some embodiments, the coated substrate or article of the invention (e.g. a film article) is stretched in at least one direction. In some embodiments, the film of the invention is stretched along a longitudinal axis of the film (also used herein as Machine Direction Orientation). In some embodiments, stretching ratio is between 1:2 to 1:7, between 1:2 to 1:3, between 1:3 to 1:7, between 1:4 to 1:7, between 1:5 to 1:7, including any range between.

In some embodiments, the coating layer is an antimicrobial coating, synergistic antimicrobial coating, antibiofilm coating, bacteriostatic coating, fungicidal coating, fungistatic coating, pesticide coating, antiviral coating, or any combination thereof.

In some embodiments, the coating layer is configured to release an effective amount of the active agent to an ambient. In some embodiments, the coating layer is any of: an antimicrobial coating, synergistic antimicrobial coating, antibiofilm coating, bacteriostatic coating, fungicidal coating, fungistatic coating, pesticide coating, antiviral coating, plant controlling coating, growth or ripening stimulating coating, growth or ripening delaying coating, or any combination thereof.

In some embodiments, the coated substrate further comprises an additional layer on top of the coating layer. In some embodiments, the additional layer provides a release barrier for the active agent. In some embodiments, the additional layer reduces or prevents release of the active agent from the coating layer, and/or from the article of the invention. In some embodiments, the additional layer is gas impermeable.

In some embodiments, the outer surface of the coating layer is further bound to the additional layer, wherein the additional layer is substantially gas impermeable. In some embodiments, the additional layer is removable. In some embodiments, the additional layer is degradable/biodegradable, and/or bioerodible. In some embodiments, the additional layer (gas impermeable layer) protects/covers at least a part of the outer surface of the coating layer. In some embodiments, the gas impermeable layer forms a contiguous layer covering essentially all the surface of the coating layer, substantially preventing a release of the active agent to the ambient.

As used herein “gas impermeable coating” refers to a coating layer capable of preventing the release or decreasing the rate of release of a gas. As used herein “gas” refers to any gas including water vapor. In some embodiments, the gas impermeable layer is a gas and/or water barrier, for reduction or prevention of gas and/or water diffusion from the coated substrate.

In some embodiments, the additional layer is characterized by a transmission rate of the active agent of at most 5, at most 2, at most 1, at most 0.5, at most 0.1 [g/m²/24 h], including any range between.

In some embodiments, the additional layer comprises a polymer, a protein, a wax, a resin, a metal, and any derivative or combination thereof.

As used herein, “wax” refers to a low melting organic mixture, or a compound of high molecular weight that is a solid at lower temperatures and a liquid at higher temperatures, and when in solid form can form a barrier (e.g., to water). Examples of waxes include animal waxes, vegetable waxes, mineral waxes, petroleum waxes, and synthetic waxes. In some embodiments, the additional coating layer comprises paraffin wax. In some embodiments, the additional coating layer comprises paraffin wax and a surfactant.

In some embodiments, the additional layer comprises PVA. In some embodiments, the additional layer comprises a biopolymer, such as polypeptides, proteins, polysaccharides and fatty acids (and esters thereof), including fibrin, fibrinogen, collagen, elastin, chitosan, gelatin, starch, glycosaminoglycans such as hyaluronic acid; copolymers of the above, or any combination thereof.

In some embodiments, the additional layer comprises a polymer selected from a polyolefin, a polyol, or any combination or a co-polymer thereof. In some embodiments, the polyol comprises polyvinyl alcohol (PVA), ethylene vinyl alcohol copolymer (EVOH), or both. In some embodiments, polyethylene comprises low-density polyethylene (LDPE), linear low-density polyethylene (LLDPE), very-low-density polyethylene (VLDPE), ultra-low-density polyethylene (ULDPE), medium-density polyethylene (MDPE), high-density polyethylene (HDPE) or any combination thereof. In some embodiments, the additional coating layer comprises polyethylene, polypropylene, polyvinyl fluoride, polyvinylidene polyfluoride, ethylene tetrafluoride, propylene hexafluoride copolymer, saponified ethylene-vinyl acetate copolymers, or any combination thereof. Hydrogel-based article

In one aspect of the invention, there is provided an article comprising the coated substrate of the invention. In some embodiments, the article is selected from a film, a ribbon, a package, a wound dressing, a plant article (e.g. plant pot, plant tray). In some embodiments, the article comprises one or more walls, wherein at least one wall comprising the coated substrate of the invention.

In one aspect of the invention, there is provided an article comprising the hydrogel of the invention. In some embodiments, the article comprises one or more walls, wherein at least one wall comprising the hydrogel of the invention.

In some embodiments, the article is selected from a film, a ribbon, a package, a wound dressing, a plant article (e.g. plant pot, plant tray) is a container. In some embodiments, the article is a plant article.

In some embodiments, any one of the articles disclosed herein is selected from transparent plastic surfaces, lenses, a package (e.g., food package, medical device package, agricultural package, and biological sample package), microelectronic device, a microelectromechanic device, a photovoltaic device, a microfluidic device, a medical device, a textile, a construction element (e.g., paints, walls, windows, handles). In some embodiments, the article according to the invention may be any optical article, such as a screen, a glazing for the automotive industry or the building industry, a mirror, an optical lens, or an ophthalmic lens. Exemplary articles include, but are not limited to, medical devices, organic waste processing device, fluidic device, an agricultural device, a package (e.g., a food packaging), a sealing article, a fuel container, a water and cooling system device and a construction element.

In some embodiments, the article is a planting container. In some embodiments, the article is a pharmaceutical composition comprising the hydrogel, a therapeutically effective amount of the active agent incorporated therewithin, and optionally a pharmaceutically acceptable carrier. In some embodiments, the article is a pharmaceutical composition being in a form of an injectable composition (e.g. a flowable composition suitable for injection to a subject, wherein the flowable composition undergoes cross-linking in situ, so as to result in a solid composition upon application thereof); a solid composition formulated for local or topical administration to a subject (e.g. in a form of a suppository for rectal, a vaginal, or urethral administration). In some embodiments, the injectable composition is for topical administration or for sub-cutaneous administration, to a subject in need thereof. In some embodiments, the injectable composition is for administration on top of a damaged tissue (e.g., a wound a lesion, etc.) of the subject (e.g. for wound treatment or prevention, and or for sealing or healing a damaged tissue of the subject). In one embodiment, a tissue sealant is formed in-situ upon cross-linking of the injectable composition on or within the application site. A tissue sealant, in some embodiments thereof, is a protectant or palliative to a local tissue at the point of introduction. In some embodiments, the sealant is a carrier of a therapeutic agent for systemic and/or local action.

As used herein, the term “suppository” relates to a solid body of various weights and shapes, adapted for introduction on a skin, or on a damaged tissue (e.g., a wound a lesion, etc.), or into the rectal, vaginal, or urethral orifice of the human body. In some embodiments, the suppository softens, and/or at least partially degrades upon exposure to a tissue of the subject at the application site. A suppository, in some embodiments thereof, is a protectant or palliative to a local tissue at the point of introduction. In some embodiments, the suppository is a carrier of a therapeutic agent for systemic and/or local action.

In some embodiments, a suppository is a rectal suppository, a vaginal suppository, or urethral suppository.

In some embodiments, the article comprising the hydrogel of the invention, wherein the hydrogel is in a form of a layer and comprises PVA and PVP at a w/w PVA:PVP ratio between about 15:1 and about 1:1; a water content of the hydrogel is between 1 and 85%, between 1 and 90%, between 10 and 85%, between 50 and 85%, between 10 and 90%, between 10 and 50%, between 20 and 50%, between 10 and 40%, between 20 and 90%, between 40 and 85%, between 60 and 85%, between 10 and 60%, between 10 and 50%, between 60 and 70%, between 70 and 85%, between 50 and 70%, between 60 and 90% w/w, including any range or value therebetween.

In some embodiments, the hydrogel of the invention and/or the article/composition comprising thereof is substantially devoid of unbound or un-complexed water.

In some embodiments, the hydrogel further comprises an active agent incorporated therewithin, wherein the active agent and the concentration thereof within the hydrogel is as described herein. In some embodiments, the hydrogel comprises a single layer, or a plurality of layers. In some embodiments, the hydrogel is in a form of a continuous layer, such as a film. In some embodiments, the term “layer”, refers to a substantially homogeneous substance of substantially uniform-thickness. In some embodiments, each layer has a different physical structure and/or a different chemical composition. In some embodiments, each layer has the same physical structure and/or the same chemical composition. In some embodiments, the term “layer”, refers to a polymeric layer. In some embodiments, the hydrogel is a continuous layer. In some embodiments, the article is a continuous film.

In some embodiments, the hydrogel or at least one wall of the article comprising thereof (e.g. a film or a sheet) is characterized by a thickness between 1 μm and 500 μm, 1 μm and 200 μm, 1 μm and 100 μm, 2 μm and 500 μm, 2 μm and 200 μm, 5 μm and 500 μm, 5 μm and 200 μm, between 10 and 20 μm, between 10 and 100 μm, between 10 and 200 μm, between 20 and 40 μm, between 40 and 50 μm, between 50 and 60 μm, between 60 and 70 μm, between 70 and 80 μm, between 80 and 90 μm, between 90 and 100 μm, between 10 and 500 μm, between 100 and 200 μm, between 200 and 500 μm, including any range or value therebetween. Each possibility represents a separate embodiment of the invention.

In some embodiments, the hydrogel or at least one wall of the article comprising thereof (e.g. a plant article, or a container) is characterized by a thickness between 0.1 and 50 mm, between 0.1 and 20 mm, between 0.5 and 50 mm, between 0.5 and 20 mm, between 0.1 and 10 mm, between 0.5 and 10 mm, including any range or value therebetween.

In some embodiments, the term “thickness” refers to the dry thickness, as described herein. In some embodiments, the hydrogel further comprises an active agent, wherein the active agent is as described herein, and a w/w concentration of the active agent within the and/or within the article is between 0.01 and 20%, including any range between as described hereinabove.

In some embodiments, the term “hydrogel” refers to a solid comprising a supramolecular structures of self-assembled polymeric molecules (i.e. PVA and PVP macromolecules) and water. In some embodiments, the supramolecular structures physically bind water molecules. In some embodiments, the supramolecular structures are in a form of a three-dimensional network of polymeric molecules. In some embodiments, the polymers are homogenously distributed (e.g., dispersed) within the hydrogel, and are substantially devoid of precipitation. In some embodiments, the hydrogel is in a form of a biphasic mixture, or in a form of a polymeric matrix stably bound to the water molecules. In some embodiments, the hydrogel is in a form of a bi-continuous phase (e.g. comprising a layered polymeric matrix and water molecules located in the interphase between the layers), or in a form of a dispersion, such as a colloidal mixture (e.g., polymers distributed within the aqueous phase). In some embodiments, the hydrogel is in a form of amorphous gel, semi crystalline gel, crystalline gel, or hydro colloid gel.

In some embodiments, the pH of the hydrogel is at a range from 5 to 8, from 5 to 7, from 5.5 to 7.5, from 6 to 7.5, from 6.5 to 7, from 7 to 8, from 5.5 to 6, from 5 to 6.5 including any range or value therebetween. Each possibility represents a separate embodiment of the invention.

In some embodiments, the polymers within the hydrogel (e.g. PVA) are at least partially cross-linked. In some embodiments, crosslinked is via a covalent cross-link. In some embodiments, crosslinked is via a physical (non-covalent) cross-link. In some embodiments, crosslinked is via a cross-linking agent. In some embodiments, the cross-linking agent is covalently bound to two or more polymeric chains (e.g. PVA chains), also referred to herein as a covalent cross-linking agent. In some embodiments, the cross-linking agent is non-covalently (e.g. via complexation interactions, via electrostatic interaction, etc.) bound to two or more polymeric chains (e.g. PVA chains), also referred to herein as a non-covalent cross-linking agent.

In some embodiments, the term “cross-linking” as used herein refers to the formation of a chemical bond between two chemical moieties or groups. In some embodiments, cross-linking comprises inter cross-linking (e.g. wherein the chemical moieties are distinct polymeric chains). In some embodiments, cross-linking comprises intra cross-linking (e.g. wherein the chemical moieties are within the same polymeric chain).

In some embodiments, the cross-linking agent (e.g. the covalent cross-linking agent) is a polyfunctional cross-linking agent comprising a plurality of moieties capable of reacting with PVA (e.g. with hydroxy groups of PVA). In some embodiments, the plurality of moieties is capable of forming covalent bond with the hydroxy groups of PVA. In some embodiments, the plurality of moieties are selected from carbonyl, active ester (e.g. NHS-ester), sulfonyl chloride, or any combination thereof. In some embodiments, the cross-linking agent (e.g. the covalent cross-linking agent) comprises a dialdehyde, or a polyaldehyde (e.g. polyglutaraldehyde). In some embodiments, the cross-linking agent is selected from boric acid and glutaraldehyde.

In some embodiments, the cross-linking agent (e.g. the non-covalent cross-linking agent) comprises a plurality of moieties capable of forming a non-covalent bond, such as a complexation interaction, electrostatic interaction, etc. In some embodiments, the cross-linking agent (e.g. the non-covalent cross-linking agent) is or comprises boric acid.

In some embodiments, the cross-linked hydrogel comprises at least partially cross-linked PVA. In some embodiments, w/w concentration of the cross-linking agent within the cross-linked hydrogel is between 0.1 and 20%, between 0.1 and 10%, between 0.5 and 20%, between 0.5 and 10%, between 0.1 and 1%, between 0.5 and 3%, between 0.5 and 5%, between 5 and 20%, between 5 and 10%, including any range between.

In some embodiments, w/w ratio between the cross-linking agent and PVA is between 0.1:1 and 1.5:1, between 0.1:1 and 0.5:1, between 0.1:1 and 0.3:1, between 0.1:1 and 0.8:1, between 0.1:1 and 1:1, between 1:1 and 1.5:1, including any range between.

In some embodiments, the hydrogel consists essentially of the polymers (also referred to herein as the polymeric content), water, and optionally comprises the active agent and/or the crosslinking agent.

In some embodiments, the polymeric content of the hydrogel is essentially composed of PVA and PVP. In some embodiments, the w/w ratio of the polymeric content relative to the total weight of the hydrogel is between 10 and 80%, between 10 and 30%, between 10 and 20%, between 10 and 50%, between 10 and 40%, between 10 and 60%, between 10 and 70%, between 5 and 40%, between 5 and 30%, between 5 and 20%, between 5 and 50%, between 5 and 60%, between 5 and 70%, including any range between.

In some embodiments, the hydrogel is a cross-linked hydrogel comprising PVA (e.g. at least partially cross-linked PVA), PVP, and the cross-linking agent, wherein the cross-linking agent and a w/w concentration thereof within the hydrogel are as disclosed herein; and wherein the hydrogel comprises a w/w ratio between PVA and PVP of between about 15:7 and about 15:4, of between about 15:7 and about 15:5, of between about 15:7 and about 15:6, or about 15:6, including any range between. Without being limited to any particular thereof, it is postulated (based on the experimental data obtained by the inventors) that an article (e.g. a layered article such as a sheet or a film) having the w/w PVA:PVP ratio between about 15:7 and about 15:4, of between about 15:7 and about 15:5, of between about 15:7 and about 15:6, or about 15:6 is characterized by enhanced mechanical strength, as compared to a similar article having a different w/w PVA:PVP ratio.

In some embodiments, the hydrogel further comprises an additive, as described herein.

In some embodiments, the article is an antimicrobial article, comprising an antimicrobial effective amount of the active agent, as described herein.

In some embodiments, the article is substantially stable (e.g. substantially retains its physical properties, mechanical strength, and/or substantially retains the initial content of the active agent) upon prolonged storage under conditions comprising (i) a temperature ranging between −40° C. and 70° C., between −40° C. and 40° C., between −40° C. and 30° C., between −40° C. and 0° C., between 0° C. and 70° C., between 0° C. and 30° C., between 30° C. and 70° C., 30° C. and 50° C., between 50° C. and 70° C., including any range between; (ii) moisture between 0 and 90%, or between 0 and 50%, including any range between and (iii) exposure to atmospheric conditions, such as an ambient atmosphere, and ambient pressure for a time period of at least 1 m, at least 2 m, at least 3 m, at least 4 m, at least 5 m, at least 7 m, at least 10 m, at least 1 y, at least 2 y, including any range between.

In some embodiments, the article is configured to substantially release the active agent incorporated therewithin. In some embodiments, the release rate is substantially constant, or is characterized by an initial burst release, and a subsequent release characterized by a substantially constant release rate. In some embodiments, the release rate is predetermined by: (i) crosslinking degree; (ii) concentration of the active agent; and/or by the crystallinity degree of the hydrogel. In some embodiments, the release rate is reduced by increasing the concentration of the cross-linking agent (or by applying any other mean for increasing cross-linking degree, such as pH, temperature, reaction time, etc.), or by performing repetitive free-thawing cycles, during the molding/shaping of the hydrogel.

In some embodiments, the article is configured to substantially release the active agent incorporated therewithin upon exposure to the ambient, wherein substantially is between 50 and 99%, between 50 and 70%, between 70 and 80%, between 80 and 90%, between 90 and 95%, between 95 and 99%, between 99 and 99.9%, between 90 and 100% by weight of the initial active agent content including any range between. In some embodiments, the hydrogel or the article of the invention comprising thereof is characterized by a gradual or sustained release profile, as opposed to a burst release profile. The terms sustained and burst release are well-known in the art. Exemplary release profiles of the articles of the invention are described in the Examples section.

In some embodiments, the release of the active agent is induced by a trigger, such as by contacting the article with soil and/or water. In some embodiments, the trigger comprises irrigation, exposure to the ambient including ambient temperature, rain, moisture, UV and/or visible light irradiation or any combination thereof. In some embodiments, the trigger is or comprises open field conditions, such as a growing plant, soil and/or area under cultivation, soil microbiome or a combination thereof.

In some embodiments, the article of the invention is configured to substantially release the active agent within a time ranging between 1 day(d) and 1 month, between 1 and 5 d, 1 and 10 d, 5 and 10 d, 10 and 20 d, 1 and 20 d, 1 h and 5 d, 1 h and 10 d, 20 and 30 d, 30 and 60 d, from 0.5 to 12 months, from 0.5 to 1 month, from 1 to 2 month, form 2 to 3 month, from 3 to 4 month, from 4 to 5 month, from 5 to 7 month, from 7 to 10 month, from 10 to 12 months, from 12 to 24 months(m), at least 1 m, at least 2 m, at least 6 m, including any range between.

In some embodiments, the article is a plant stimulator configured to control (e.g. reduce or increase) the growth of the plant and/or a plant part (such as fruit, foliage, root, stem, etc.). In some embodiments, the article is configured to release an effective amount of the plant stimulator to an ambient, so as to control the growth of the plant and/or a plant part. In some embodiments, the effective amount is sufficient for stimulating (or increasing) or inducing plant growth.

In some embodiments, the effective amount is sufficient for any one of: increasing, inducing, accelerating, or delaying ripening. In some embodiments, the plant stimulator comprises a ripening accelerator (e.g. ethylene, etc.), or a ripening inhibitor. In some embodiments, the plant stimulator comprises a natural or synthetic plant stimulating agent (e.g. dormancy breaking agent, etc.). In some embodiments, the plant stimulator comprises a plant growth hormone. In some embodiments, reducing, increasing, accelerating, or delaying as used herein, refers to at least 10%, at least 20%, at least 30%, at least 50%, at least 80%, at least 100%, at least 200%, at least 300%, at least 500%, at least 1000%, at least 10.000% increase or decrease in the growth, yield, and/or ripening of the plant or of the plant part, including any range between, as compared to a similar plant which has not been exposed to the active agent or to the article of the invention.

In some embodiments, the article is stable at a temperature between −25 and 80° C., between −25 and 75° C., between −25 and 0° C., between 0 and 10° C., between 0 and 80° C., between 0 and 50° C., between 0 and 75° C., between 10 and 80° C., between 10 and 75° C., including any range or value therebetween.

In some embodiments, the article is stable upon exposure to UV and/or visible light radiation. In some embodiments, the article is stable for at least 12 months, for at least 15 months, for at least 18 months, for at least 20 months, at least 24 months upon exposure to UV radiation of 180 kilo Langley per year (KLy p.a.). In some embodiments, UV stability of the article is measured according to ISO 4892-2.

As used herein the term “stable” refers to the capability of the article to maintain its structural and/or mechanical integrity. In some embodiments, the article is referred to as stable, if the article is characterized by a mechanical integrity sufficient to be used as a packaging material. In some embodiments, the article is referred to as stable, if the article substantially maintains its structural and/or mechanical integrity under outdoor conditions such as a temperature -25 and 75° C., rain, moisture, UV and/or visible light irradiation for a time period of at least 12 months, as described hereinabove. In some embodiments, the stable article is rigid under outdoor conditions. In some embodiments, the stable article at least 50% of its initial tensile strength and/or elasticity. In some embodiments, the term “initial” is immediately after the manufacturing of the article prior to exposure to the storage conditions and/or outdoor conditions. In some embodiments, substantially is as described hereinbelow.

In some embodiments, the article (e.g. in a form of a sheet, a ribbon, a thread or a film) is characterized by elongation at break between 10 and 1000%, between 10 and 20%, between 20 and 30%, between 30 and 40%, between 40 and 50%, between 50 and 60%, between 50 and 100%, between 10 and 100%, between 60 and 100%, between 70 and 100%, between 80 and 100%, between 100 and 1000%, between 100 and 200%, between 200 and 300%, between 300 and 400%, between 400 and 500%, between 500 and 1000%, between 100 and 500%, between 500 and 700%, between 700 and 1000%, including any range or value therebetween.

In some embodiments, the article (e.g. in a form of a sheet, a ribbon, a thread or a film) is characterized by tensile strength at a break between 5 and 50 N/10 mm, between 5 and 10 N/10 mm, between 10 and 50 N/10 mm, between 10 and 20 N/10 mm, between 20 and 30 N/10mm, between 30and 35 N/10 mm, between 35 and 40 N/10 mm, between 40 and 45 N/10 mm, between 45 and 50 N/10 mm, including any range or value therebetween.

In some embodiments, the article (e.g. in a form of a sheet, a ribbon, a thread or a film) is characterized by tensile strength (e.g. abrasion stability, tear stability, peel off stability, etc.) sufficient for use thereof as a plant article, a packaging article, or both. In some embodiments, the article (e.g. in a form of a sheet, a ribbon, a thread or a film) is characterized by tensile strength up to about 5 MPa, up to about 10 MPa, up to about 50 MPa, up to about 1 MPa, up to 0.5 MPa, up to 0.85 MPa, up to about 5 MPa, including any range between.

In some embodiments, the article (e.g. in a form of a sheet, a ribbon, a thread or a film) is characterized by tensile strength up to about 5 MPa, up to about 10 MPa, up to about 50 MPa, up to about 1 MPa, up to 0.5 MPa, up to 0.85 MPa, up to about 5 MPa, including any range between, wherein the article consists essentially of the hydrogel of the invention (e.g. devoid of the polymeric substrate in contact with the hydrogel). In some embodiments, the article comprising a w/w ratio between PVA and PVP of between about 15:7 and about 15:4, of between about 15:7 and about 15:5, of between about 15:7 and about 15:6, or about 15:6, including any range between, is characterized by enhanced mechanical strength, as compared to a similar article having a w/w ratio between PVA and PVP of below 15:4.

In some embodiments, the article (e.g. in a form of a sheet, a ribbon, a thread or a film) is characterized by elasticity sufficient for use thereof as a plant article, a packaging article, or both. In some embodiments, the article is characterized by sufficient elasticity so as to obtain any predefined shape. In some embodiments, the article (e.g. in a form of a sheet, a ribbon, a thread or a film) is characterized by elasticity sufficient for obtaining the shape of the wrapped matter (such as an edible matter, a package, a crop material, etc.). In some embodiments, the article (e.g. in a form of a sheet, a ribbon, a thread or a film) substantially retains its shape for a time period disclosed herein.

In some embodiments, the article of the invention is characterized by visible light transmission (VLT) between 80% and 99%, between 82% and 99%, between 85% and 99%, between 89% and 99%, between 90% and 99%, between 95% and 99%, between 80% and 95%, between 82% and 95%, between 85% and 95%, between 89% and 95%, between 90% and 95%, between 80% and 90%, between 82% and 90%, or between 85% and 90%, including any range therebetween. Each possibility represents a separate embodiment of the invention. As used herein “visible light transmission (VLT)” refers to a measurement of the amount of visible light waves that transmit through a material.

In some embodiments, the article of the invention is characterized by a haze between 6% and 20%, between 8% and 20%, between 10% and 20%, between 12% and 20%, between 15% and 20%, between 8% and 16%, between 10% and 16%, or between 6% and 10%, including any range therebetween. Each possibility represents a separate embodiment of the invention. In some embodiments, the article of the invention e is characterized by a haze between 6% and 20%, between 8% and 20%, between 10% and 20%, between 12% and 20%, between 15% and 20%, between 8% and 16%, between 10% and 16%, or between 6% and 10%, including any range therebetween. Each possibility represents a separate embodiment of the invention. As used herein, “haze” refers to the fraction of light transmission which deviates greater than 2.5°.

In some embodiments, the article of the invention is characterized by a shrinkage between 1% and 40%, between 1% and 35%, between 1% and 30%, between 1% and 25%, between 1% and 20%, 5% and 40%, between 5% and 35%, between 5% and 30%, between 5% and 25%, between 5% and 20%, 10% and 40%, between 10% and 35%, between 10% and 30%, between 10% and 25%, or between 10% and 20%, including any range therebetween, obtained by thermal shrinkage process. Each possibility represents a separate embodiment of the invention. As used herein, “thermal shrinkage” refers to the process of reheating a plastic film or sheet, thereby changing its linear dimension.

In some embodiments, the article of the invention substantially retains (e.g. at least 90%, at least 95% retention, or more) its properties such as physical and/or structural stability and/or light transparency, upon deformation (e.g. shrinkage up to 30% or more), as compared to the undeformed article.

In some embodiments, the article of the invention comprising the coating layer bound to the polymeric layer substantially retains the mechanical properties of the polymeric layer. In some embodiments, the article of the invention comprising the coating layer stably bound to the polymeric layer, wherein stably bound refers to the adhesion strength of the coating layer to the polymeric layer being in a range between 0.1 and 10N, between 0.2 and 10N, between 0.3 and 10N, between 0.5 and 10N, between 0.1 and 1N, between 0.5 and IN, between 1 and 10N, between 1 and 5N, including any range between.

Adhesion strength may be determined by any one of the well-known peel strength tests.

In some embodiments, stably bound refers to the adhesion strength sufficient for preventing separation of the coating layer from the polymeric layer upon exposure thereof to (i) regular stress conditions applied during utilization of the article (e.g. upon wrapping an edible matter, wrapping a bale, wrapping a container, mounting or installing an agricultural plant protecting film); and/or (ii) upon exposure thereof to the ambient conditions, as described herein.

In some embodiments, the article of the invention comprising the coating layer bound to the polymeric layer, increases a mechanical strength (such as tensile strength) of the polymeric layer by at least 10%, by at least 20%, by at least 30%, by at least 50%, by at least 70%, by at least 100%, between 10 and 100%, or any value therebetween.

In some embodiments, there is provided a method comprising applying an effective amount of the article of the invention to a plant, to a plant part, or to an area under cultivation, thereby (i) reducing or eradicating a pest, (ii) preventing or reducing a plant disease associated with the pest, or both (i) and (ii). In some embodiments, the effective amount is a pesticide effective amount. In some embodiments, the effective amount is a growth stimulating effective amount. In some embodiments, the effective amount is a ripening acceleration or ripening delaying effective amount.

In some embodiments, reducing comprises at least 10% reduction, at least 50% reduction, at least 100% reduction, at least 1000% reduction, at least 10.000% reduction, as compared to a control plant or to an area under cultivation, which has not been treated by the composition of the invention.

In some embodiments, the effective amount comprises between 10 g and 1000 kg, between 10 g and 100 g, between 10 g and 1000 g, between 100 g and 10 kg, between 100 g and 1000kg, between 1 and 100 kg, between 10 and 100 kg, between 10 and 1000 kg of the active agent per 1 ha of the area under cultivation.

In some embodiments, applying is performed one or more times during the cultivation cycle (e.g. 1, 2, 3, 4, 5, 10, or any range or value between), at any one of the cultivation stages selected from, plating, seeding, harvesting, pre-planting, post planting, pre-seeding, post-seeding, pre-harvesting, post-harvesting, or at the storage of the plant or a plant part, optionally wherein the plant part comprises a fruit, a seed, a leave, a stem, a root, or a combination thereof.

In some embodiments, the article of the invention is applying by providing the article of the invention in close proximity to the plant, to the plant part, and/or to the area under cultivation.

Beads

In another aspect, there is provided a composition comprising a plurality of beads, each bead comprises the cross-linked hydrogel of the invention, wherein the cross-linked hydrogel of the invention further comprises an active agent incorporated within the hydrogel; a w/w concentration of the active agent within the hydrogel is between 0.5 and 20%; and wherein a w/w concentration of the cross-linking agent within the hydrogel or within the bead is between 0.5 and 10%. In some embodiments, the active agent and the concentration thereof within the hydrogel is as described herein. In some embodiments, the water content of the hydrogel is between 70 and 90%, between 1 and 90%, between 1 and 10%, between 10 and 85%, between 50 and 85%, between 10 and 90%, between 10 and 50%, between 20 and 50%, between 10 and 40%, between 20 and 90%, between 40 and 85%, between 60 and 85%, between 10 and 60%, between 10 and 50%, between 60 and 70%, between 70 and 85%, between 50 and 70%, between 60 and 90% w/w or any value therebetween.

In some embodiments, the composition is substantially devoid of unbound or un-complexed water.

In some embodiments, the cross-linking agent is as described herein.

In some embodiments, the beads and/or the composition comprise a w/w PVA:PVP ratio between about 5:1 and about 1:1, between about 4:1 and about 1:1, between about 3:1 and about 1:1, between about 3:1 and about 2:1, between about 2:1 and about 1:1, including any value therebetween. In some embodiments, the beads comprise a w/w PVA:PVP ratio between about 3:1 and about 1:1, and between 0.5 and 5%, or between 0.5 and 10% w/w of boric acid as a cross-linking agent, and further comprising the active agent as described herein.

In some embodiments, the beads and/or the composition further comprise between 0.1 and 5%, between 0.1 and 1%, between 1 and 5% w/w of alginic acid and/or a salt thereof (e.g. Na-alginate). In some embodiments, the beads and/or the composition further comprise between 0.01 and 3% of calcium ions, and/or a calcium salt (e.g. CaCl₂).

In some embodiments, the beads are substantially spherically shaped. In some embodiments, the beads are solid.

In some embodiments, the beads are characterized by an average cross-section between 0.1 and 100 mm, between 0.1 and 50 mm, between 1 and 100 mm, between 1 and 50 mm, between 50 and 100 mm, between 1 and 10 mm, between 5 and 100 mm, between 5 and 50 mm, including any range or any value therebetween.

In some embodiments, the plurality of beads is characterized by a particle size distribution (PDI) between 1 and 1.9, between 1 and 1.5, between 1.2 and 1.9, between 1.2 and 1.5, between 1.2 and 1.8, between 1 and 1.3, between 1 and 1.4, between 1 and 1.2, including any range or any value therebetween.

In some embodiments, the beads have a spherical geometry or shape. In some embodiments, the beads have an inflated or a deflated shape. In some embodiments, a plurality of beads is devoid of any characteristic geometry or shape. In some embodiments, the beads have a spherical shape, an elliptical shape, a quasi-spherical shape, a quasi-elliptical sphere, a deflated shape, a concave shape, an irregular shape, or any combination thereof.

In some embodiments, the plurality of beads are substantially spherically shaped, wherein substantially is as described herein. In some embodiments, the plurality of beads are substantially elliptically shaped, wherein substantially is as described herein. One skilled in the art will appreciate that the exact shape of each of the plurality of beads may differ from one bead to another. Moreover, the exact shape of the beads may be derived from any of the geometric forms listed above, so that the shape of the bead does not perfectly fits to a specific geometrical form. One skilled in the art will appreciate that the exact shape of the beads may have substantial deviations (such as at least 5%, at least 10%, at least 20% deviation) from a specific geometrical shape (e.g., a sphere or an ellipse).

The “average cross-section” of a plurality of beads is the arithmetic average of the average cross-sections (e.g. diameters) of each of the beads. In some embodiments, average cross-section refers to the cross-section of the beads in a dry state. Those of ordinary skill in the art will be able to determine the average diameter (or other characteristic dimension) of a plurality of beads (e.g. using a caliper, or any other suitable technique).

In some embodiments, the beads or the composition comprising thereof is configured to substantially release the active agent incorporated therewithin. In some embodiments, the release rate is substantially constant, or is characterized by an initial burst release, and a subsequent release characterized by a substantially constant release rate. In some embodiments, the release rate is predetermined by: (i) crosslinking degree; (ii) concentration of the active agent; and/or by the crystallinity degree of the hydrogel. In some embodiments, the release rate is reduced by increasing the concentration of the cross-linking agent.

In some embodiments, the beads or the composition comprising thereof is configured to substantially release the active agent incorporated therewithin upon exposure to the ambient, wherein substantially is between 50 and 99%, between 50 and 70%, between 70 and 80%, between 80 and 90%, between 90 and 95%, between 95 and 99%, between 99 and 99.9%, between 90 and 100% by weight of the initial active agent content including any range between. In some embodiments, the beads or the composition comprising thereof is characterized by a gradual or sustained release profile, as opposed to a burst release profile. The terms sustained and burst release are well-known in the art. Exemplary release profiles of the articles of the invention are described in the Examples section.

In some embodiments, the release of the active agent is induced by a trigger, such as by contacting the article with soil and/or water. In some embodiments, the trigger comprises irrigation, exposure to the ambient including ambient temperature, rain, moisture, UV and/or visible light irradiation or any combination thereof. In some embodiments, the trigger is or comprises open field conditions, such as a growing plant, soil and/or area under cultivation, soil microbiome or a combination thereof.

In some embodiments, the beads or the composition comprising thereof is configured to substantially release the active agent within a time ranging between 1 day(d) and 1 month, between 1 and 5 d, 1 and 10 d, 5 and 10 d, 10 and 20 d, 1 and 20 d, 1 h and 5 d, 1h and 10 d, 20 and 30 d, 30 and 60 d, from 0.5 to 12 months, from 0.5 to 1 month, from 1 to 2 month, form 2 to 3 month, from 3 to 4 month, from 4 to 5 month, from 5 to 7 month, from 7 to 10 month, from 10 to 12 months, from 12 to 24 months(m), at least 1 m, at least 2 m, at least 6 m, including any range between.

In some embodiments, the coating layer is an antimicrobial coating, synergistic antimicrobial coating, antibiofilm coating, bacteriostatic coating, fungicidal coating, fungistatic coating, pesticide coating, antiviral coating, or any combination thereof.

In some embodiments, the coating layer is configured to release an effective amount of the active agent to an ambient. In some embodiments, the coating layer is any of: an antimicrobial coating, synergistic antimicrobial coating, antibiofilm coating, bacteriostatic coating, fungicidal coating, fungistatic coating, pesticide coating, antiviral coating, plant controlling coating, growth or ripening stimulating coating, growth or ripening delaying coating, or any combination thereof.

In some embodiments, the composition is a pesticide composition configured to control (e.g. reduce) or prevent plant pathogen related diseases; and/or plant and/or a plant part infestation by the pest, and/or pest loading on or within the plant and/or a plant part or at the are under cultivation. In some embodiments, the composition is configured to release a pesticide effective amount of the biocide to an ambient, so as to control or prevent any one of: pest loading, plant pathogen related diseases, plant and/or a plant part infestation by the pest.

In some embodiments, the composition is a plant stimulating composition configured to control (e.g. reduce or increase) the growth of the plant and/or a plant part (such as fruit, foliage, root, stem, etc.). In some embodiments, the composition is configured to release an effective amount of the plant stimulator to an ambient, so as to control the growth of the plant and/or a plant part. In some embodiments, the effective amount is sufficient for stimulating (or increasing) or inducing plant growth.

In some embodiments, the effective amount is sufficient for any one of: increasing, inducing, accelerating, or delaying ripening. In some embodiments, the plant stimulator comprises a ripening accelerator (e.g. ethylene, etc.), or a ripening inhibitor. In some embodiments, the plant stimulator comprises a natural or synthetic plant stimulating agent (e.g. dormancy breaking agent, etc.). In some embodiments, the plant stimulator comprises a plant growth hormone.

In some embodiments, the effective amount (e.g. antimicrobial effective amount, or plant stimulating effective amount) of the active agent is between 1 and 1000 ppm, between 1 and 10 ppm, between 10 and 20 ppm, between 15 and 20 ppm, between 20 and 30 ppm, between 10 and 30 ppm, between 10 and 100 ppm, between 10 and 50 ppm, between 50 and 100 ppm, between 50 and 70 ppm, between 70 and 100 ppm, between 100 and 1000 ppm, between 1 and 500 ppm, including any range or value therebetween.

In some embodiments, the effective amount refers to a concentration of the active agent (e.g. vapor concentration in the air, or w/w concentration in soil or in the plant or plant part) at a specific location such as at the immediate surroundings of the plant, plant part, and/or an edible matter, or at the area under cultivation (e.g. soil, plant or plant part).

In some embodiments, the terms “antimicrobial effective amount” and “biocide effective amount” are used herein interchangeably.

In some embodiments, reducing, increasing, accelerating, or delaying as used herein, refers to at least 10%, at least 20%, at least 30%, at least 50%, at least 80%, at least 100%, at least 200%, at least 300%, at least 500%, at least 1000%, at least 10.000% increase or decrease in the growth, yield, and/or ripening of the plant or of the plant part, including any range between, as compared to a similar plant which has not been exposed to the active agent or to the composition of the invention.

### Agricultural Compositions

In another aspect, there is provided a composition comprising an aqueous solvent and a plurality of colloidal particles dispersed therewithin, dispersed within the aqueous solution, wherein: each of the plurality of colloidal particles comprises PVA and PVP at a w/w PVA:PVP ratio between about 15:1 and about 1:1; each of the plurality of colloidal particles further comprises a surfactant and optionally an active agent.

In some embodiments, the colloidal particles or the composition comprises a w/w PVA:PVP ratio between about 15:1 and about 1:1, about 10:1 and about 1:1, about 8:1 and about 1:1, about 5:1 and about 1:1, between about 4:1 and about 1:1, between about 3:1 and about 1:1, between about 3:1 and about 2:1, between about 2:1 and about 1:1, including any value therebetween. In some embodiments, the colloidal particles comprise (i) a w/w PVA:PVP ratio between about 5:1 and about 1:1, or about 5:1 and about 1:1, (ii) between 0.5 and 5%, or between 0.5 and 10% w/w of boric acid as a cross-linking agent, and further comprising the active agent as described herein.

In some embodiments, the PVA and PVP are characterized by a MW as described herein.

In some embodiments, the composition is a liquid dispersion, a liquid emulsion, or a liquid suspension.

In some embodiments, a w/w concentration of the active agent within the composition is between 0.5 and 20%; and wherein a w/w concentration of the cross-linking agent (e.g. boric acid) within the composition is between 0.1 and 10%, between 0.3 and 5%, between 0.1 and 5%, between 5 and 10% including any range or any value therebetween.

In some embodiments, the active agent and the concentration thereof within the composition is as described hereinabove.

In some embodiments, an average particle size of the plurality of colloidal particles is between 0.2 and 20 μm, between 0.2 and 1 μm, between 0.2 and 10 μm, between 1 and 20 μm, between 1 and 10 μm, including any range or any value therebetween. The particle size can be established by any suitable means known in the art such as DLS.

In some embodiments, the plurality of colloidal particles is characterized by a particle size distribution (PDI) between 1 and 1.9, between 1 and 1.5, between 1.2 and 1.9, between 1.2 and 1.5, between 1.2 and 1.8, between 1 and 1.3, between 1 and 1.4, between 1 and 1.2, including any range or any value therebetween.

In some embodiments, the composition is substantially devoid of aggregates. In some embodiments, a w/w concentration of the surfactant within the composition is between 0.1 and 10%, between 0.2 and 5%, between 0.5 and 5%, between 0.3 and 5%, between 5 and 10% w/w, including any range or any value therebetween.

In some embodiments, the surfactant is selected from the group consisting of a non-ionic surfactant, an anionic surfactant, a cationic surfactant and an amphoteric surfactant or any combination thereof.

Non-limiting examples of anionic surfactants include but are not limited to: (C₆-C₈)alkyl-sulfate and/or sulfonate (e.g., sodium or potassium lauryl sulfate, sodium or potassium dodecyl sulfate), fatty alcohol ether sulfate salt, polyacrylate (e.g., sodium or potassium polyacrylates), or any combination thereof.

Non-limiting examples of non-ionic surfactants include but are not limited to: alkyl-polyglycoside (e.g., Triton CG 110, APG 810), polyethyleneglycol-(C₁₁-C₁₅)alkyl-ether, alkoxylated fatty alcohol, alkoxylated fatty acid, glucosyl dialkyl ether, polysorbate, span, tween, a polyether, a polyol, a polysaccharide, a polypeptide, a polyester, polyvinyl acetate, polyacrylamide, and polyacrylate, including any mixture or a copolymer thereof.

In one embodiment, the alkoxylated fatty acid comprises ethoxylated castor oil.

In one embodiment, the anionic surfactant is selected from the group consisting of alkyl benzene sulfonate, alcohol ether sulfate, secondary alkane sulfonates and alkyl sulfates including any combination thereof. In some embodiments, the surfactant of the invention is or comprises a non-ionic surfactant. In some embodiments, the non-ionic surfactant of the invention is or comprises alkoxylated fatty acid, glucosyl dialkyl ether, polysorbate, span, tween, a polyether, a polyol, a polysaccharide, a polypeptide, a polyester, polyvinyl acetate, polyacrylamide, and polyacrylate, including any mixture or a copolymer thereof. Other non-ionic surfactants are well-known in the art. In some embodiments, the non-ionic surfactant of the invention comprises a polymeric surfactant (e.g. a dispersant). Numerous dispersants are well-known in the art. In some embodiments, alkoxylated fatty acid comprises ethoxylated castor oil.

Non-limiting examples of nonionic surfactants include, but are not limited to, polysorbate (e.g., polysorbate 20, 40, 60, and 80), tween (e.g., tween 20, 40, 60, and 80), ethoxylated castor oil, narrow-range ethoxylate, octaethylene glycol monododecyl ether, pentaethylene glycol monododecyl ether, nonoxynols, triton X-100, polyethoxylated tallow amine, cocamide monoethanolamine, cocamide diethanolamine, poloxamers, glycerol monostearate, glycerol monolaurate, sorbitan monolaurate, sorbitan monostearate, sorbitan tristearate, decyl glucoside, lauryl glucoside, octyl glucoside, lauryldimethylamine oxide, dimethyl sulfoxide, phosphine oxide, and others. The term “anionic surfactant” refers to any surfactant containing an anionic functional group including sulfate, sulfonate, phosphate, and carboxylates.

Non-limiting examples of anionic surfactants include, but are not limited to, alkylbenzenesulfonate, ammonium lauryl sulfate, sodium lauryl sulfate (sodium dodecyl sulfate, SLS, or SDS), sodium laureth sulfate (sodium lauryl ether sulfate or SLES), sodium myreth sulfate, dioctyl sodium sulfosuccinate (Docusate), perfluorooctanesulfonate (PFOS), perfluorobutanesulfonate, alkyl-aryl ether phosphates, alkyl ether phosphates, sodium stearate, sodium lauroyl sarcosinate, perfluorononanoate, and perfluorooctanoate (PFOA or PFO). In some embodiments, the anionic surfactant is a linear alkylbenzenesulfonate. In some embodiments, the surfactant is or comprises Tween 80.

In some embodiments, the cross-linking agent is as described herein.

In some embodiments, the composition further comprises between 0.1 and 5%, between 0.1 and 1%, between 1 and 5% w/w of alginic acid and/or a salt thereof (e.g. Na-alginate). In some embodiments, the beads and/or the composition further comprise between 0.01 and 3% of calcium ions, and/or a calcium salt (e.g. CaCl₂).

In some embodiments, the colloids are substantially spherically shaped. In some embodiments, the colloids are substantially uniform, and wherein the entire colloidal particle is substantially solid (e.g. devoid of a liquid core).

In some embodiments, a w/w concentration of PVA within the composition is between 5 and 20%, between 5 and 10%, between 10 and 20%, between 10 and 15%, between 15 and 20%, or about 15% w/w, including any range or any value therebetween.

In some embodiments, the composition is characterized by dispersion stability of at least 3 months, at least 2 months, at least 6 months, at least 8 months, at least 12 months, between 1 and 12 months, or more including any range or any value therebetween.

In some embodiments, the composition is configured to substantially release the active agent incorporated therewithin. In some embodiments, the release rate is substantially constant, or is characterized by an initial burst release, and a subsequent release characterized by a substantially constant release rate. In some embodiments, the release rate is predetermined by: (i) crosslinking degree; (ii) concentration of the active agent. In some embodiments, the release rate is reduced by increasing the concentration of the cross-linking agent.

In some embodiments, the composition is configured to substantially release the active agent incorporated therewithin upon exposure to the ambient, wherein substantially is between 50 and 99%, between 50 and 70%, between 70 and 80%, between 80 and 90%, between 90 and 95%, between 95 and 99%, between 99 and 99.9%, between 90 and 100% by weight of the initial active agent content including any range between.

In some embodiments, the release of the active agent is induced by a trigger, such as by contacting the article with soil and/or water. In some embodiments, the trigger comprises irrigation, exposure to the ambient including ambient temperature, rain, moisture, UV and/or visible light irradiation or any combination thereof. In some embodiments, the trigger is or comprises open field conditions, such as a growing plant, soil and/or area under cultivation, soil microbiome or a combination thereof.

In some embodiments, the composition is configured to substantially release the active agent within a time ranging between 1 day(d) and 1 month, between 1 and 5 d, 1 and 10 d, 5 and 10 d, 10 and 20 d, 1 and 20 d, 1 h and 5 d, 1h and 10 d, 20 and 30 d, 30 and 60 d, from 0.5 to 12 months, from 0.5 to 1 month, from 1 to 2 month, form 2 to 3 month, from 3 to 4 month, from 4 to 5 month, from 5 to 7 month, from 7 to 10 month, from 10 to 12 months, from 12 to 24 months(m), at least 1 m, at least 2 m, at least 6 m, including any range between.

In some embodiments, the composition of the invention is an antimicrobial composition, synergistic antimicrobial composition, antibiofilm composition, bacteriostatic composition, fungicidal composition, fungistatic composition, pesticide composition, antiviral composition, or any combination thereof.

In some embodiments, the composition is configured to release an effective amount of the active agent to an ambient. In some embodiments, the composition is any of: an antimicrobial composition, synergistic antimicrobial composition, antibiofilm composition, bacteriostatic composition, fungicidal composition, fungistatic composition, pesticide composition, antiviral composition, plant controlling composition, growth or ripening stimulating composition, growth or ripening delaying composition, or any combination thereof.

In some embodiments, the composition is a pesticide composition configured to control (e.g. reduce) or prevent plant pathogen related diseases; and/or plant and/or a plant part infestation by the pest, and/or pest loading on or within the plant and/or a plant part or at the are under cultivation. In some embodiments, the composition is configured to release a pesticide effective amount of the biocide to an ambient, so as to control or prevent any one of: pest loading, plant pathogen related diseases, plant and/or a plant part infestation by the pest.

In some embodiments, the composition is a plant stimulating composition configured to control (e.g. reduce or increase) the growth of the plant and/or a plant part (such as fruit, foliage, root, stem, etc.). In some embodiments, the composition is configured to release an effective amount of the plant stimulator to an ambient, so as to control the growth of the plant and/or a plant part. In some embodiments, the effective amount is sufficient for stimulating (or increasing) or inducing plant growth.

In some embodiments, the effective amount is sufficient for any one of: increasing, inducing, accelerating, or delaying ripening. In some embodiments, the plant stimulator comprises a ripening accelerator (e.g. ethylene, etc.), or a ripening inhibitor. In some embodiments, the plant stimulator comprises a natural or synthetic plant stimulating agent (e.g. dormancy breaking agent, etc.). In some embodiments, the plant stimulator comprises a plant growth hormone.

In some embodiments, the effective amount (e.g. antimicrobial effective amount, or plant stimulating effective amount) of the active agent is between 1 and 1000 ppm, between 1 and 10 ppm, between 10 and 20 ppm, between 15 and 20 ppm, between 20 and 30 ppm, between 10 and 30 ppm, between 10 and 100 ppm, between 10 and 50 ppm, between 50 and 100 ppm, between 50 and 70 ppm, between 70 and 100 ppm, between 100 and 1000 ppm, between 1 and 500 ppm, including any range or value therebetween.

In some embodiments, the effective amount refers to a concentration of the active agent (e.g. vapor concentration in the air, or w/w concentration in soil or in the plant or plant part) at a specific location such as at the immediate surroundings of the plant, plant part, and/or an edible matter, or at the area under cultivation (e.g. soil, plant or plant part).

In some embodiments, the terms “antimicrobial effective amount” and “biocide effective amount” are used herein interchangeably.

In some embodiments, reducing, increasing, accelerating, or delaying as used herein, refers to at least 10%, at least 20%, at least 30%, at least 50%, at least 80%, at least 100%, at least 200%, at least 300%, at least 500%, at least 1000%, at least 10.000% increase or decrease in the growth, yield, and/or ripening of the plant or of the plant part, including any range between, as compared to a similar plant which has not been exposed to the active agent or to the composition of the invention.

In some embodiments, the composition is an agricultural composition comprising an agriculturally acceptable carrier. In some embodiments, the composition is formulated for spraying or fogging.

In some embodiments, there is provided a method comprising applying an effective amount of the composition (e.g. the agricultural composition) to a plant, to a plant part, or to an area under cultivation, thereby (i) reducing or eradicating a pest, (ii) preventing or reducing a plant disease associated with the pest, or both (i) and (ii). In some embodiments, the effective amount is a pesticide effective amount. In some embodiments, the effective amount is a growth stimulating effective amount. In some embodiments, the effective amount is a ripening acceleration or ripening delaying effective amount.

In some embodiments, reducing comprises at least 10% reduction, at least 50% reduction, at least 100% reduction, at least 1000% reduction, at least 10.000% reduction, as compared to a control plant or to an area under cultivation, which has not been treated by the composition of the invention.

In some embodiments, the effective amount comprises between 10 g and 1000 kg, between 10 g and 100 g, between 10 g and 1000 g, between 100 g and 10 kg, between 100 g and 1000 kg, between 1 and 100 kg, between 10 and 100 kg, between 10 and 1000 kg of the composition (e.g. a liquid composition, or a solid composition comprising the hereindisclosed beads) per 1 ha of the area under cultivation.

In some embodiments, applying is performed one or more times during the cultivation cycle (e.g. 1, 2, 3, 4, 5, 10, or any range or value between), at any one of the cultivation stages selected from, plating, seeding, harvesting, pre-planting, post planting, pre-seeding, post-seeding, pre-harvesting, post-harvesting, or at the storage of the plant or a plant part, optionally wherein the plant part comprises a fruit, a seed, a leave, a stem, a root, or a combination thereof.

In some embodiments, the composition is the liquid composition, and applying comprises any of immersion, coating, irrigating, dipping, spraying, fogging, scattering, painting, injecting, or any combination thereof.

In some embodiments, the composition is a solid composition (e.g. comprises the beads) and applying is performed (i) providing the composition in close proximity to the plant, to the plant part; (ii) applying the composition to a soil; or both (i) and (ii). In some embodiments, the composition is a solid composition and applying is by providing a container (e.g. a gas permeable container) comprising an effective amount of the composition in close proximity to the plant, to the plant part, and/or to area under cultivation.

In another aspect, there is provided a method comprising providing or exposing an edible matter to any one of the compositions and/or articles of the invention.

In some embodiments, providing or exposing is performed once. In some embodiments, providing or exposing is repeated one or more times. In some embodiments, providing or exposing is performed at one or more stages in a life-cycle of the edible matter (such as seeding, foliage, flowering, post-harvest, pre-harvest etc.). In some embodiments, the pesticidal composition is applied to a harvested fruit and/or vegetable. In some embodiments, any one of the compositions and/or articles is applied to a processed fruit and/or vegetable, wherein processed comprises any food processing technique, such as cooking, slicing, etc.

In some embodiments, the method is for (i) reducing edible matter decay; and/or (ii) reducing pathogen load of the edible matter. In some embodiments, the method is for controlling pathogen load (e.g. fungal load) on or within the edible matter.

In some embodiments, “reducing” as used herein throughout is as compared to a non-treated edible matter. In some embodiments, “controlling” as used herein throughout comprises reducing colony forming units (CFU) of the pathogen on or within the edible matter/plant or plant part/area under cultivation by a factor of at least 10,000, of at least 100,000, of at least 1,000,000, including any value or arrange therebetween, wherein reducing is as compared to a non-treated edible matter.

As used herein, the terms “controlling” and “reducing” are used interchangeably and are related to reduction of colony forming unit (CFU), as compared to a non-treated control, by a factor of between 2 and 10, between 10 and 100, between 100 and 1000, between 1000 and 10,000, between 10,000 and 100,000, between 100,000 and 1,000,000, including any range between.

In some embodiments, the method is for reducing pathogenic activity on or within the edible matter. As used herein, the term “reducing pathogenic activity” refers to the ability to inhibit, prevent, reduce or retard bacterial growth, fungal growth, biofilm formation or eradication of living bacterial cells, or their spores, or fungal cells or viruses in a suspension, on or within the edible matter, at the specific location, or any combination thereof. In some embodiments, inhibition or reduction or retardation of biofilm formation by a pathogen positively correlates with inhibition or reduction or retardation of growth of the pathogen and/or eradication of a portion or all of an existing population of pathogens.

In some embodiments, the method of the invention comprises reducing CFU/cm2 on the surface of the edible matter by at least by a factor of 10, at least by a factor of 30, at least by a factor of 50, at least by a factor of 60, at least by a factor of 65, at least by a factor of 70, at least by a factor of 100, at least by a factor of 200, at least by a factor of 400, at least by a factor of 800, at least by a factor of 1000, at least by a factor of 10,000, at least by a factor of 100,000, at least by a factor of 1,000,000, as compared to a non-treated edible matter surface.

In some embodiments, the method of the invention comprises reducing CFU on or within the edible matter at least by a factor of 10, at least by a factor of 30, at least by a factor of 50, at least by a factor of 60, at least by a factor of 65, at least by a factor of 70, at least by a factor of 100, at least by a factor of 200, at least by a factor of 400, at least by a factor of 800, at least by a factor of 1000, at least by a factor of 10,000, at least by a factor of 100,000, at least by a factor of 1,000,000, as compared to a non-treated edible matter.

In some embodiments, the method of the invention comprises inhibiting or eradicating pathogen load on or within the edible matter, wherein inhibiting or eradicating comprise complete arrest of pathogen growth and/or complete eradication of the initial pathogen load.

Colonies start as single pathogen (CFU) which multiplies and forms a colony. Given enough CFUs close by, eventually, neighboring colonies will fuse. Increasing the magnification allows detection of micro-colonies before they fuse. In some embodiments, “colony” as used herein, refer to a colony observed by the naked eye.

In some embodiments, the method is for preventing pathogen infection of the edible matter at a storage temperature of below 30° C., below 25° C., below 20° C., below 10° C., below 5° C., during a time period of at least 1 month (m), at least 1 month (m), at least 2 m, at least 3 m, at least 4 m, at least 5 m, at least 6 m, at least 7 m, at least 8 m, at least 10 m, at least 12 m, including any range or value therebetween.

In some embodiments, the method is for prolonging shelf life of the edible matter, compared to the untreated edible matter. In some embodiments, prolonging is for a time period ranging from 1 to 100 days, from 1 to 10 days, from 1 to 60 days, from 10 to 20 days, from 20 to 40 days, from 40 to 60 days, from 60 to 100 days, including any range between.

In some embodiments, the method is for selectively reducing fungal activity on or within the edible matter, wherein reducing is as described hereinabove. In some embodiments, the method is for selectively reducing or preventing fungal activity.

In some embodiments, the method of the invention is for reducing edible matter decay. In some embodiments, edible matter decay comprises decay related to the pathogen load of the edible matter. In some embodiments, edible matter decay comprises decay related to common biological processes occurring within the harvested edible mater, such as dehydration, cell death, etc. As used herein, the term “reducing” comprises decay reduction of the edible matter treated by a sanitizing composition of the invention, as compared to a non-treated edible matter, wherein reduction is by a factor of between 2 and 10, between 10 and 100, between 100 and 1000, between 1000 and 10,000, including any range between.

In some embodiments, the method is for enhancing or prolonging storage stability and/or extending shelf life, relative to untreated edible matter. In some embodiments, enhancing or prolonging is by at least 20%, at least 50%, at least 100%, at least 200%, at least 500%, at least 1000%, including any range between.

In some embodiments, edible matter decay is selected from the group consisting of: loss from pathogen load, decomposing, sprouting, loss from a disease, rotting, dehydration, and blackheart formation, loss from a higher organism or any combination thereof.

In some embodiments, the edible matter is selected from the group consisting of fruits, vegetables, grains, sprouts, nuts, seeds, meats, meat products, milk, milk products, fish, poultry, eggs, and mixtures thereof.

Non-limiting example of edible matter include but are not limited to: apple, avocado, citrus (e.g. clementine, orange, grapefruit, lemon), date, kiwi, lychee, mango, peach, pear, persimmon, pomegranate, pepper, asparagus, banana, broccoli, cabbage, carrot, cauliflower, celery, corn, kohlrabi, cucumber, eggplant, garlic, lettuce, onion, peanut, potato, strawberry, sweet pepper, sweet potato, tomato, watermelon, grains (e.g. wheat, barley, etc.), dry fruits (almonds, nuts, etc.) and grape or any combination thereof.

Definitions

As used herein, the term “pest” and the term “pathogen” are used herein interchangeably and refer herein to a microorganism. In some embodiments, the pathogen is or comprises one or more plant pathogens. In some embodiments, the pathogen comprises a pest (e.g., an insect, mite, a nematode and/or a gastropod mollusk). In some embodiments, the pathogen comprises a fungus, a bacterium, or both.

Non-limiting example of plant pathogens include but are not limited to: cryophiles, nematodes, mites, ticks, fungi, algae, mold, bacteria, virus, spores, yeast, and bacteriophages or any combination thereof.

In some embodiments, the pathogen is selected from the group consisting of: bacteria, a fungus, a yeast, a virus, an algae, a mold, protozoa, an amoeba, and spore-propagating microorganisms or any combination thereof.

In some embodiments, bacteria are selected from the group consisting of gram-positive bacteria. In some embodiments, the gram-positive bacteria are selected from the group consisting of Staphylococcus, Streptococcus, Enterococcus, Bacillus, Corynebacterium, Nocardia, Clostridium, Actinobacteria and Listeria or any combination thereof.

In some embodiments, bacteria are selected from the group consisting of gram-negative bacteria. In some embodiments, the gram-negative bacteria are selected from the group consisting of Escherichia, Salmonella, Shigella, Enterobacteriaceae, Pseudomonas, Moraxella, Helicobacter, Stenotrophomonas, Bdellovibrio, acetic acid bacteria, Legionella, cyanobacteria, spirochaetes, green sulfur bacteria, green non-sulfur bacteria, and respiratory symptoms Moraxella or any combination thereof.

In some embodiments, bacteria are selected from the group consisting of Escherichia coli, Staphylococcus aureus, Pseudomonas aeruginosa and Enterococcus hiraeor any combination thereof.

In some embodiments, the fungus is selected from the group consisting of Magnaporthe, Ophiostoma, Cryphonectria, Fusarium, Ustilago, Alternaria, Cochliobolus, Aspergillus, Candida, Cryptococcus, Histoplasma, and Pneumocytis or any combination thereof.

In some embodiments, the yeast is selected from the group consisting of Cryptococcus neoformans, Candida albicans, Candida tropicalis, Candida stellatoidea, Candida glabrata, Candida krusei, Candida parapsilosis, Candida guilliermondii, Candida viswanathii, Candida lusitaniae and Rhodotorula mucilaginosa or any combination thereof.

In some embodiments, the virus is selected from the group consisting of Adenoviruses, Herpesviruses, Poxviruses, Parvoviruses, Reoviruses, Picornaviruses, Togaviruses, Orthomyxoviruses, Rhabdoviruses, Retroviruses and Hepadnaviruses or any combination thereof.

The disclosed agricultural compositions set forth above may be formulated in any manner. Non-limiting formulation examples include but are not limited to Dried grains, Emulsifiable concentrates (EC), Wettable powders (WP), Soluble liquids (SL), Aerosols, Ultra-low volume concentrate solutions (ULV), Soluble powders (SP), Microencapsulation, Water dispersed granules (WDG), Flowables (FL), Microemulsions (ME), Nano-emulsions (NE), etc. In any formulation described herein, percent of the active ingredient is well within the skills of the artisan e.g., within a range of 0.01% to 99.99%.

In some embodiments, the composition is in the form of, but not limited to, a liquid, gel, solid or biofumigant. In some embodiments, the composition comprises a surfactant to be used for the purpose of emulsification, dispersion, wetting, spreading, integration, disintegration control, stabilization of active ingredients, and improvement of fluidity or rust inhibition. In some embodiments, the surfactant is a non-phytotoxic non-ionic surfactant. In one embodiment, the carrier is a perlite particle.

As used herein, the term “pharmaceutically acceptable” means approved by a regulatory agency or listed in the U.S. Pharmacopeia or other generally recognized pharmacopeia for use in animals, and more particularly in humans.

As used herein, the term “carrier” refers to a diluent, adjuvant, excipient, or a vehicle administered together with the active ingredient. In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

In some embodiments, carriers are sterile liquids such as water-based liquids; oils, including those of petroleum, animal, vegetable or synthetic origin, such as peanut oil, soybean oil, mineral oil, sesame oil and the like; solvents such as polyethylene glycols, glycerin, propylene glycol, ethanol or other synthetic solvents. In some embodiments, the composition further comprises sodium benzoate.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone.

In some embodiments, suitable pharmaceutical excipients include starch, glucose, lactose, sucrose, gelatin, malt, rice, flour, chalk, silica gel, salts (e.g., sodium chloride, sodium stearate, and glycerol monostearate), talc, dried skim milk, glycerol, propylene glycol, water, ethanol and the like. The composition may further comprise wetting and/or emulsifying agents, or pH buffering agents such as acetates, citrates or phosphates.

In some embodiments, preservatives such as benzyl alcohol, benzoic acid (including any salt thereof, such as sodium benzoate) and/or methyl parabens; antioxidants (such as ascorbic acid, propyl gallate, tocopherols, tertiary butylhydroquinone, butylated hydroxyanisole, sodium pyrosulfite, potassium pyrosulfite, and butylated hydroxytoluene, or sodium bisulfite); and agents for the adjustment of tonicity (such as sodium chloride or dextrose) are also envisioned.

In some embodiments, the carrier comprises, in total, from about 0.1% to about 99.99999% by weight of the pharmaceutical composition presented herein.

In some embodiments, the carrier comprises any one the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

Other non-limiting examples of carriers for include but are not limited to: cocoa butter (relates to a mixture of triglycerides of saturated and unsaturated fatty acids, such as stearic, palmitic, oleic, lauric, and linoleic acids), a cocoa butter substitute (relates to vegetable oils modified by esterification, hydrogenation, etc., including hydrogenated vegetable oil and hard fat), glycerinated gelatin, a polyethylene glycol-based carrier, and a surfactant (such as polyoxyethylene sorbitan fatty-acid esters and polyoxyethylene stearates) or any combination thereof.

Non-limiting examples of carriers for pharmaceutical compositions being in the form of a cream include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the pharmaceutical optionally comprises a thickener.

Non-limiting examples of thickeners include, but are not limited to microcrystalline cellulose, a starch, a modified starch, gum tragacanth, gelatin, and a polymeric thickener (e.g. polyvinylpyrrolidone) or any combination thereof.

In some embodiments, the pharmaceutical composition further comprises a pharmaceutical agent such as an anti-inflammatory agent, an analgesic, an antimicrobial agent or any combination thereof.

Non-limiting examples of analgesics include but are not limited to: methyl salicylate, menthol, camphor, eucalyptol, capsicum, ibuprofen, aspirin, paracetamol, and rofecoxib or any pharmaceutically acceptable salts or mixtures thereof.

Non-limiting examples of anti-inflammatory agents include but are not limited to: non-steroidal anti-inflammatory agents such as celecoxib, and etoricoxib, diclofenac, fenoprofen, flurbiprofen, ketoprofen, naproxen, etodolac, and diflunisal or any pharmaceutically acceptable salts or mixtures thereof.

Non-limiting examples of antimicrobial agents include but are not limited to: beta-lactam antibiotics, aminoglycoside antibiotics, tetracycline antibiotics, trimethoprim antibiotics, nitrofurantoin antibiotics and pharmaceutically acceptable salts thereof, and mixtures thereof.

In some embodiments, the pharmaceutical composition further comprises an additive. Non-limiting examples of additives include, but are not limited to: a thickener, a filler, a colorant, and an excipient or any combination thereof.

In some embodiments, the pharmaceutical composition is in a form of an enema composition. In some embodiments, an enema composition is an aqueous composition. In some embodiments, an aqueous composition is in a form of a solution, a dispersion, or a suspension.

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

### General

As used herein the term “about” refers to +10%. Further, all numerical values, e.g. when referring the amounts or ranges of the elements constituting the formulation are approximations which are varied (+) or (−) by up to 10% of from the stated values. It is to be understood, even if not always explicitly stated that all numerical designations are preceded by the term “about”.

The terms “comprises”, “comprising”, “includes”, “including”, “having” and their conjugates mean “including but not limited to”.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure. The term “consisting essentially of” is used to define formulations which include the recited elements but exclude other elements that may have an essential significance on the formulation.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict. The word “optionally” and the word “further” are used herein interchangeably.

The terms, film/films and layer/layers are used herein interchangeably. As used herein, the term “coat” refers to the combined layers disposed over the substrate, excluding the substrate, while the term “substrate” refers to the part of the composite structure supporting the disposed layer/coating. In some embodiments, the terms “layer”, “film” or as used herein interchangeably, refer to a substantially uniform-thickness of a substantially homogeneous substance.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In some embodiments, the terms “locus” and “area under cultivation” are used herein interchangeably meaning a habitat, plant, seed, material, or plant environment such as air, soil, rhizome, etc. In some embodiments, the terms “locus” refers to a plant and/or to a part of the plant (e.g., a leaf). In some embodiments, the term “reducing”, or any grammatical derivative thereof, indicates that at least 20%, 30%, 40%, 50%, 60%, 70%, 80%, 90%, or more, reduction of growth or even complete growth inhibition in a given time as compared to the growth in that given time of the pathogen not being exposed to the treatment as described herein. In some embodiments, the term “completely inhibited”, or any grammatical derivative thereof, refers to 100% arrest of growth in a given time as compared to the growth in that given time of the pathogen not being exposed to the treatment as described herein. In some embodiments, the terms “completely inhibited” and “eradicated” including nay grammatical form thereof, are used herein interchangeably.

Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1

In an exemplary non-limiting procedure, The PVA/PVP hydrogels were prepared by co-solution method and treated with repeated freezing-thawing recycle which improves their mechanical properties. In our lab. we prepared PVA/PVP hydrogel is by heating PVA aqueous solution, e.g., 80-95° C., followed by addition of PVP and then cooling down to room temperature. During the cooling process the hydrogel formation leads to gradual increased viscosity which enable to mold the formed hydrogel to any desired shape6. The mechanical properties of the final material may be improved, as mentioned previously by repeated freezing-thawing cycles and/or crosslinking. Through careful modification of the processing parameters during freeze-thaw cycling, the degree of crystallinity, or physical cross-linking, can be controlled allowing for tailoring of the hydrogel's mechanical properties. Phase separation into a PVA/PVP-rich phase and a water-rich phase is a crucial and unique mechanism that occurs during freeze-thawed PVA/PVP hydrogel. During exposure to cold temperatures water freezes, expelling PVA and PVP and forming regions of high PVA and PVP concentration. As the PVA and PVP chains come into close contact with each other, crystallite PVA formation and hydrogen bonding occur. These interactions remain intact following thawing and create a non-degradable three-dimensional hydrogel network.

The composite material was analyzed by infrared spectroscopy, X-ray diffraction, differential scanning calorimeter. Its water absorption, mechanical property and cytotoxicity were also tested. The results show that PVP can chemically bond with PVA and form composite hydrogel with PVA in molecular level. The content of PVP can affect the structure, crystallinity, glass transition temperature, water absorption and mechanical property of PVA hydrogel. The PVA/PVP hydrogel has high water content and good cytocompatibility. It is a permeable material similar to natural cartilage, which is a promising artificial cartilage material.

The general trend is that water content decreases with PVP content of the PVA/PVP hydrogel increasing. Water content of burns dressing should be about 60 percent, but that of PVA/PVP hydrogel, the highest reaches close to 90 percent; water absorption of PVA/PVP hydrogel increases with PVP content decreasing when PVA content is 15 percent, water absorption of all the hydrogels are up to 120%, the highest is close to 220%; pH value of PVA/PVP hydrogel is in the range from 6.5 to 6.8, it is higher than 5.5 and closes to 7, but also the highest value, 6.83, is most close to value 7. The tensile strength of PVA/PVP hydrogel notably increases with PVP content increasing, it reaches to the highest value, up to 0.85 MPa, when PVP content is 6 percent.

### MATERIALS AND METHODS

### Materials

Poly(vinyl alcohol), M_{w} 89,000-98,000, 99+% hydrolyzed, mono and diammonium phosphite, and trichloroacetic acid (TCA), benzoyl peroxide, thymol≥98.5%, hydrogen Peroxide 30-32% (w/w) and urea-hydrogen peroxide adduct 97% (UHP) from Sigma Aldrich, Polyvinylpyrrolidone K30, Mn 40,000 from Fluka, TREION double distilled water (DDW).

### Preparation of PVA/PVP/H₂O₂ Hydrogel by a Swelling Method

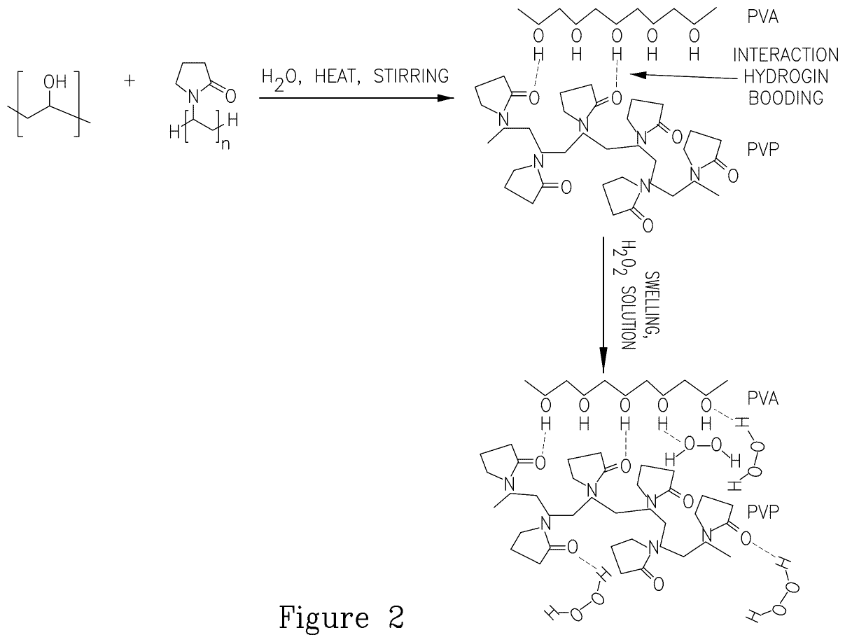

PVA/PVP/H₂O₂ hydrogel was prepared according to FIG. 2, by dissolving 7.5 gr of PVA in 39.5 gr of DDW in a 100 ml Erlenmeyer flask. The flask was placed in a silicon oil bath at 80-95° C. until complete dissolution. 3 gr of PVP was added to the PVA solution and was left to react for about 1.0 hour. The solution was then cooled to 70° C. (until the foam from the reaction disappeared). The solution was then poured into a desired mold. The obtained molded hydrogel was placed in a freezer (approximately −18° C.) overnight, then thawed (freeze/thaw cycle). The number of freeze/thaw cycles varied depending on the desired properties. Another option to improve the mechanical properties is by covalent crosslinking of the hydrogel by glutaraldehyde, or UV, or [symbol] -irradiation, or surface crosslinking with glutaraldehyde through the PVA, according to the literature²¹. The hydrogel was dried and then stored in 4° C. Swelling of the PVA/PVP with H₂O or H₂O/H₂O₂ was then accomplished by dipping the PVA/PVP hydrogel in a flask containing H₂O or 10% H₂O₂ aqueous solution, respectively for different time periods. The hydrogel was then stored in 4° C.

The water or H₂O/H₂O₂ swelling % was determined by Equation 1 where Ws denotes the sample weight after swelling and wa is the weight of the dry sample.

$$Swelling ( \% ) = 100 \times \frac{Ws - Wd}{Wd} \\ \% Swelling of PVA / PVP hydrogel . \qquad Equation 1$$

### In Situ Preparation of PVA/PVP/UHP Hydrogel

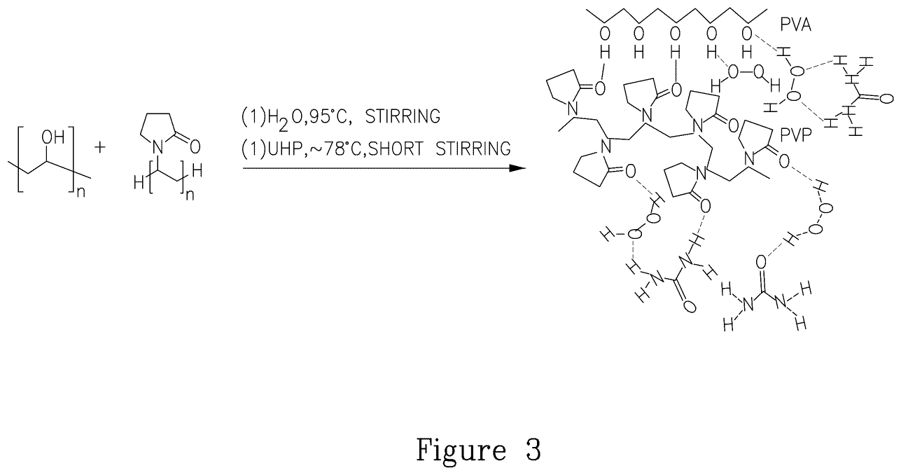

The PVA/PVP hydrogel was prepared according to FIG. 3, by dissolving 7.5 gr of PVA in 39.5 gr of DDW in a 100 ml Erlenmeyer flask. The flask was placed in a silicon oil bath at 80-95° C. until complete dissolution. 3 gr of PVP was added to the PVA solution and reacted for about 1.0 hour. The solution was cooled to 70-78° C., then urea-hydrogen peroxide (UHP) was added to the Erlenmeyer flask until complete dissolution (FIG. 3). The obtained molded hydrogel was then immediately poured into a desired mold. The obtained PVA/PVP/UHP mold was then placed in a freezer (approximately −18° C.) overnight and then thawed (freeze/thaw cycle). The number of freeze/thaw cycles varied depending on the desired hydrogel properties. The hydrogel was then stored in 4° C. If necessary, covalent cross-linking of the hydrogel to slow down the rate of H₂O₂ release will be accomplished with boric acid or glutaraldehyde through the PVA. Similar experiment was done to prepare PVA/PVP/H₂O₂ by substituting UHP for H₂O₂ and the temperature wherein UHP was added (70-78° C.) to 60-70° C. (in order to prevent significant decomposition of the H₂O₂)

PVA/PVP/UHP hydrogel water content (%) was calculated according to equation 2 where wo denotes the sample weight after preparation and wa is the weight of the dry sample.

$$Water content ( \% ) = 100 \times \frac{W 0 - Wd}{Wd} \\ Water content of PVA / PVP / UHP hydrogel . \qquad Equation 2$$

### In Situ Preparation of PVA/PVP/UHP hydrogel in Various Forms

### A. PVA/PVP/UHP Hydrogel Coatings on PE Films

7.5 gr of PVA was dissolved in 39.5 gr of DDW in a 100 ml Erlenmeyer flask. The flask was placed in a silicon oil bath at 80-95° C. until complete dissolution. 3 gr of PVP was added to the PVA solution and reacted for about 1.0 hour. The solution was cooled to 70-78° C., then the urea-hydrogen peroxide was added to the Erlenmeyer until complete dissolution. PE film surface was treated with corona instrument (350-watt·min/m², 20 scans). The hydrogel hot reaction solution was then poured over the oxidized PE film and was spread with Mayer rod technique. The coated PE was placed in a freezer (approximately −18° C.) overnight and thawed later (freeze/thaw cycle). The number of freeze/thaw cycles varied depending on the desired coating properties. The resulting coated film is demonstrated in FIG. 4.

### B. PVA/PVP/UHP Hydrogel Nursery Tray for Plants

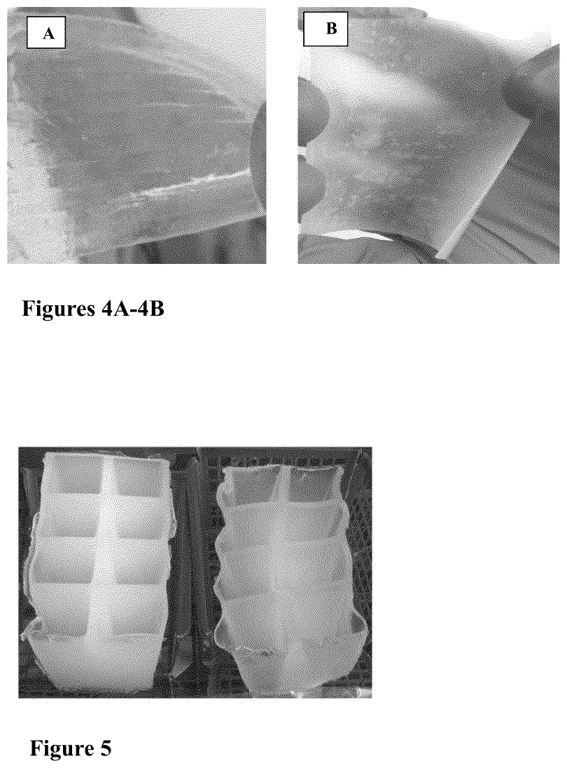

7.5 gr of PVA was dissolved in 39.5 gr of DDW in a 100 ml Erlenmeyer flask. The flask was placed in a silicon oil bath at 80-95° C. until complete dissolution. 3 gr of PVP was added to the PVA solution and reacted for 1.5 hours. The solution was cooled to 70-78° C., then the urea-hydrogen peroxide was added to the Erlenmeyer flask until complete dissolution. The hydrogel hot solution was poured into nursery trays for plant cultivation (FIG. 5). The filled pattern was kept in a freezer (approximately −18° C.) overnight and thawed later (freeze/thaw cycle), and then stored in 4° C.

### C. PVA/PVP/UHP Hydrogel Sheets and Chips

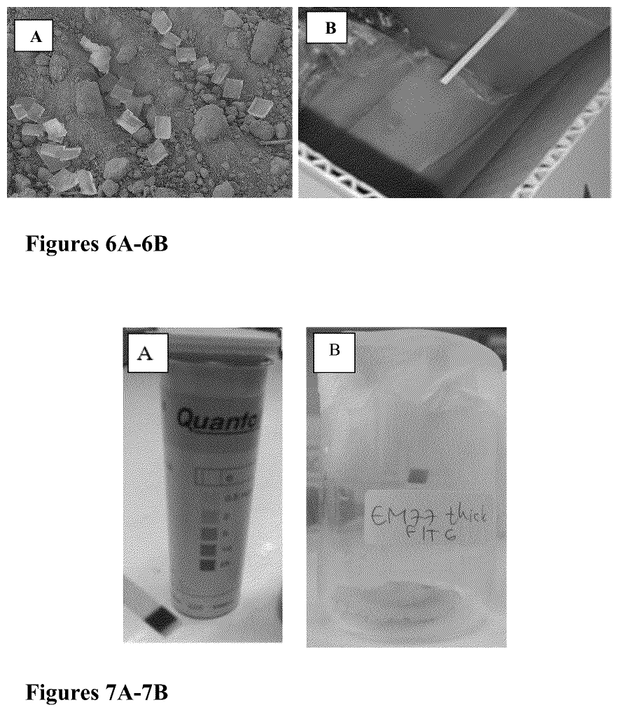

7.5 gr of PVA was dissolved in 39.5 gr of DDW in a 100 ml Erlenmeyer flask. The flask was placed in a silicon oil bath at 80-95° C. until complete dissolution. 3 gr of PVP was added to the PVA solution and reacted for 1.5 hours. The solution was cooled to 70-78° C., then the urea- hydrogen peroxide was added to the Erlenmeyer flask until complete dissolution. The hydrogel hot solution was poured into flat tray. The filled pattern was kept in a freezer (approximately −18° C.) overnight and thawed later (freeze/thaw cycle). The cured hydrogel sheet was then removed from the pattern and kept at 4° C. (FIG. 6B). These sheets were further cut into chips (FIG. 6A).

### D. PVA/PVP/UHP Hydrogel Beads

7.5 gr of PVA was dissolved in 39.5 gr of DDW in a 100 ml Erlenmeyer flask. The flask was placed in a silicon oil bath at 80-95° C. until complete dissolution. 3 gr of PVP was added to the PVA solution and reacted for 1.5 hours. The solution was cooled to 70-78° C., then the urea-hydrogen peroxide was added to the Erlenmeyer flask until complete dissolution. The PVA/PVP/UHP aqueous solution was then introduced into an heated syringe and dropwise injected into cold (4° C.) aqueous solution.

### In Situ Preparation of PVA/PVP/H₂O₂ Hydrogels in Various Forms

The previous processes describing the preparation of PVA/PVP/UHP hydrogels in various forms was repeated substituting the UHP for H₂O₂ and the cooling temperature of 70-78° C. for 60-70° C.

### CHARACTERIZATION METHODS

### Determination of Hydrogen Peroxide Concentration by KMnO₄ Titration

The method was used for both hydrogels and silica-urea coatings to determine the concentration of released H₂O₂ from the hydrogels/film to water, as follows

100-220 mg of the hydrogel samples (1×1 cm²) or 3.5×4 cm² PE/silica-urea films loaded H₂O₂ were added to a magnetic stirred vial containing 11.9 ml of DDW and 660 μl of sulfuric acid 96%. Pre-calibrated KMn04 (with sodium oxalate) titrant was then pippeted into the vial, until the solution was changed from transparent to purple.

### Determination of Hydrogen Peroxide Vapors Concentration

Mancherey-Nagel Quantofix® peroxides test sticks (FIG. 7) were used to evaluate hydrogen peroxide concentrations in gas phase. The test sticks are semi quantitative visual colorimetric indicators with wide measuring range (1-100 mg/L). The indicators react in the presence of hydrogen peroxide, in redox reactions and turns blue as immediate result. The indicator sticks are pre calibrated and accurate at the 2-9 pH range and temperature range 4-30° C. The obtained color result is compared to the color scale bar demonstrated in FIG. 7A. The indicator strips were tested on various system set ups:

A hydrogel sheet was placed on the bottom of a beaker. The Quantofixx peroxide test stick was taped to the side of the beaker (about half height) and immediately sealed with parafilm. The test was performed in various temperatures: Lab temperature (24° C.), refrigerator temperature (2-4° C.) and warm temperature in incubator at 30° C. The reading of the hydrogen peroxide vapors concentration was monitored at certain time intervals up to weeks.

A hydrogel sheet was placed on the bottom of a package containing plant cuttings. Quantofix® peroxide test sticks were taped to the package side wall. The package was stored in conditions simulating export transportation. The package was kept at 6° C. for the first 24 h then at room temperature (25° C.) for 3-4 h. The package was then place back at 6° C. for additional 24 h.

### Release Kinetics of H₂O₂ From the Hydrogels

### Release Kinetics of H₂O₂ From PVA/PVP/H₂O₂ Hydrogel and PVA/PVP/UHP Hydrogel to Water

For the experiments we used PVA/PVP/H₂O₂ hydrogel with initial size of 1×1 cm² and dry weight of 70.9 mg which was swollen with H₂O₂ aqueous solution (H₂O/H₂O₂) up to final weight of 306.5 mg containing 8.6 w % H₂O₂; PVA/PVP/UHP hydrogel with initial ize of 1×1 cm² and weight of 117 mg containing 92.4 ml urea/H₂O₂ aqueous solution (7.8% H₂O₂ and 84.6% H₂O); The thickness of the hydrogel samples was 0.2 cm, unless the thickness effect was studied. The above hydrogels were dipped in room temperature in vials containing 10 ml of DDW for different times: 5, 10, 15, 30, 60 and 90 min. For each time a different sample was used. The concentration of the released H₂O₂ in water at each time was then determined by addition to each vial 650 μl of H₂SO₄ (96%) and titrated with a pre-calibrated KMnO₄ solution as described previously.

### Release Kinetics of H₂O₂ Vapor From PVA/PVP/UHP Hydrogel

Peroxide test sticks were taped to the inside of a 100 ml beaker. A hydrogel sheet of 7×4 cm² was placed in the beaker which was then sealed with Parafilm tape. The peroxide stick color indicator (FIG. 9) was monitored over different time periods. The tests were performed in refrigerator (3° C.), room (24° C.) and warm (30° C.) temperatures. A similar procedure was performed substituting the sheets with coatings.

### Attenuated Total Reflectance (ATR) Measurements

The instruments used for ATR measurements are Bruker ALPHA-FTIR Quicksnap™ sampling module equipped with Platinum ATR diamond module (Bruker, Germany) and translated by the OPUS program.

### pH Measurements of PVA/PVP/UHP Hydrogels

pH measurements were performed using a pH test strip (MACHEREY-NAGEL pH-fix 0-14 pH) and a calibrated pH electrode (EUTECH INSTRUMENTS pH 700). The test strip is colorimetric and done by visual comparison to the compatible sequence chart.

### Light Microscope Surface Analysis

PVA/PVP/UHP hydrogel surface micro scale analysis was conducted by Images (×10) carried out by a light microscope apparatus integrated with FastScan scanning probe microscope (Bruker AXS). Image processing was done by Nanoscope analysis software.

### Atomic Force Microscopy Surface Roughness Analysis

The hydrogels surface roughness analysis was carried out using a Bio FastScan scanning probe microscope (Bruker AXS). All images were obtained using soft tapping mode with Fast Scan B (Bruker) silicon probe (spring constant of 1.8N/m). The resonance frequency of the cantilever was approximately 450 KHz (in air). The measurements were performed under environmental conditions. The images were captured in the retrace direction with a scan rate of 1.6 Hz. The resolution of the images was 512 samples/line. For image processing and roughness analysis we used Nanoscope analysis software. Before roughness analysis of the images, the “flatting” and “planefit” functions were applied to each image. The image scans were done on 5 micrometer scale. The roughness average (Ra) was used as the main parameter to distinguish between sample roughness. Sample preparation was done between pressed glass slides.

### Phytotoxicity and Activity Tests of PVA/PVP/UHP Hydrogels

To test the hydrogels phytotoxicity on plants and their efficiency against pests, different test methods were used. The tests were done in two modes:

Direct contact between the hydrogel and plant/pathogens.

Indirect contact—hydrogen peroxide vapors were released in an enclosed space with the plant/pathogens.

### Phytotoxicity Tests

Hydrogels phytotoxicity direct test patterned to a plant nursery seedling plate.

PVA/PVP/UHP and PVA/PVP/H₂O₂ hydrogel nursery plate pot trays (FIG. 5) were planted with broccoli seedlings (8 seedlings in each tray) and let grow under greenhouse. The seedlings root system and foliage were monitored daily by visual inspection for phytotoxicity. The test lasted for three weeks.

### Phytotoxicity Indirect Test of Hydrogels Patterned to Sheets

PVA/PVP/UHP and PVA/PVP/H₂O₂ hydrogel sheets (prepared as described in section 3.5) were cut to size of 19.5×23.5 cm² with a thickness of about 4 mm and covered on bottom with a protective nylon. The hydrogels were placed on the carton base with the protective nylon directed to the carton bottom and Quantofix™ peroxide test stick was added to the carton side wall (FIG. 6B). Wrapped cuttings in perforated nylon were placed on the exposed side of the hydrogel sheet. Two types of cuttings were tested: vinca and Calibrachoa. The cartons containing the cuttings, were stored in a cooling room set to 6° C. for 48 hours. The cartons were taken out of cooling at alternating times to room temperature. This was done to simulate real-world transportation logistics. After 24 hours, the cartons were opened to verify peroxide vapors concentration by comparing the stick color to the color scale (FIG. 7). After the 48 hours cooling storage, the cuttings were planted and let grow for two weeks. During growth, phytotoxic signs were monitored visually including plant roots, foliage, and plant growth density on the tray.

### Activity Against Pests

Effectiveness test of PVA/PVP/UHP and PVA/PVP/H₂O₂ hydrogels against the spread of Tomato brown rugose fruit virus (ToBRFV) on tobacco and tomato plants.

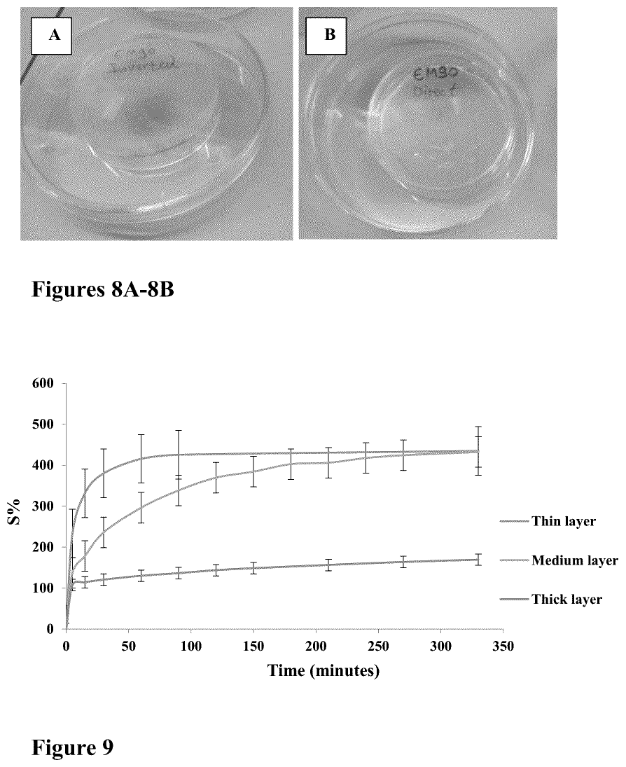

The tests were done by direct contact between the virus and hydrogel surface and by indirect contact using hydrogen peroxide vapors in an enclosed space with the virus. PVA/PVP/UHP and PVA/PVP/H₂O₂ hydrogels were poured into a 50 mm diameter petri dishes and molded to a 5 mm thickness. Five petri dishes were inoculated with 0.5 mL ToBRFV solution, in direct contact with the hydrogel and covered with an 80 mm petri dish. Another five petri dishes (50 mm diameter) were inverted with the hydrogel on the top part of each dish. 0.5 mL ToBRFV solution was pipetted onto the bottom part of each petri dish so that the virus is indirectly in contact with the hydrogel. A Quantofix™ peroxide stick was located near the virus solution. Each inverted petri dish was covered with an 80 mm petri dish top to create an enclosed environment. The test system is described in FIG. 8. Petri dishes containing PVA/PVP hydrogel were used as control samples. One Control duplicate were tested for the direct mode. The second control duplicate were tested for the inverted mode. The virus exposure time to the hydrogels liquid and gas medium was performed for a duration of 48 hours in room temperature.

1 mL of phosphate buffer (0.01M, pH˜7) extraction solution was then added to the exposed virus solution. The tomato and tobacco plants leaves were sprayed with silicon carbide (Carborundum). 3 leaves of each plant were rubbed in the direction of its arteries. This procedure injured the plants leaves enabling the penetration of the virus. 100 μl of the virus solution was introduced to each selected leaf and was rubbed on the direction of its arteries. Bioassay of the plants were measured via local lesions (LLs) count. LLs are formed due to the hypersensitivity of the tobacco plants to the ToBRFV. The virus causes the plant to activate apoptosis around the virus infection resulting in a programmed local cell death. The cell death is expressed by white lesions on the leaves. LL appearance occurred between 3-4 days after the virus infection. Each LL was counted as an infecting virus. The tomato plants ToBRFV infection is not visually conclusive through a bioassay; hence, Enzyme-Linked Immunosorbent Assay (ELISA) test was applied.

### ELISA Test Detection for ToBRFV in Tomato Plants Leaves

The tested leaves were picked from tomato plants infected with ToBRFV one month earlier. The leaves that appeared to have post disease symptom development were subjected to indirect ELISA tests. Samples were ground in a coating buffer (Agdia) and were incubated for 3 hours at 37° C. with a 1:5000 dilution of the ToBRFV antiserum. Detection was carried out by incubating the samples with AP-conjugated goat anti-rabbit (IgG)(Sigma, Steinheim, Germany) for 3 h at 37° C. P-nitro phenyl phosphate (Sigma) substrate was used at a concentration of 0.6 mg/mL. The developing color was recorded by ELISA reader (Thermo Fisher Multiskan FC) at 405 and 620 nm. The minimum ratio O.D values was determined as three times the value of the negative (healthy) controls to be considered positive.

### RESULTS

### % H₂O Absorption of the PVA/PVP/H₂O₂ Hydrogel

The absorbed water/H₂O₂ content of the PVA/PVP/H₂O₂ hydrogels was calculated for samples (1×1 cm²) with varied freeze/thaw cycles, according to the equation described in the methodology section. One sample was exposed to one freeze/thaw cycle and another sample to six freeze/thaw cycles. The % absorbed water according to equation 1 after one and six freeze/thaw cycles was 330% and 205%, respectively. The results show an inverse relation between the absorbed water content and the number of freeze/thaw (F/T) cycles. During the freeze/thaw process, phase separation was found to occur between the polymers produced the hydrogel (PVA and PVP) and the water forming regions. The polymer chains come into close contact with each other with each additional freeze/thaw cycle, forming increasing number of hydrogen bonds and possible crystallite structures which created a stronger three dimensional hydrogel network²³. These strong bonds restrict the water swelling abilities of the hydrogels. To further improve the absorbed water content of the hydrogel, different drying methods were applied prior to the swelling process: (1) Dry in an incubator set to 120° C. until the dry weight stabilizes and (2) lyophilization (freeze-drying). After each drying process, the samples were further swollen with hydrogen peroxide aqueous solution. The % absorbed water contents of the heated and lyophilized samples were 180 and 270%, respectively.

These results show a considerable increase in water swelling degree for the pre-lyophilized sample (180 to 270%) rather than high temperature heat drying. The initial maximum swelling process was performed overnight, but in order to understand the kinetics of the swelling as function of hydrogel thickness, hydrogel samples with different thickness (0.1, 0.25 and 1.2 cm) were prepared, as described in FIG. 9.

FIG. 9 exhibits kinetics dependence of the swelling process on hydrogel thickness. The swelling time saturation of thin and medium layer samples were reached at approximately 1.5 and 5.5 h, respectively. The thick layer sample did not reach swelling saturation during the test time range. Therefore, sample layer thickness has an impact on swelling saturation time in that thinner and medium hydrogel samples require shorter swelling saturation time. This can be explained by the hydrogel surface area exposed to the aqueous environment. Thinner layer samples have a higher surface area exposed to the aqueous environment allowing for easier excess of water molecules to penetrate into the hydrogel. These results are still not clear enough and further research is required.

### Release Kinetics of H₂O₂ From the Hydrogels to Water

### Release Kinetics of H₂O₂ From PVA/PVP/H₂O₂ Hydrogels

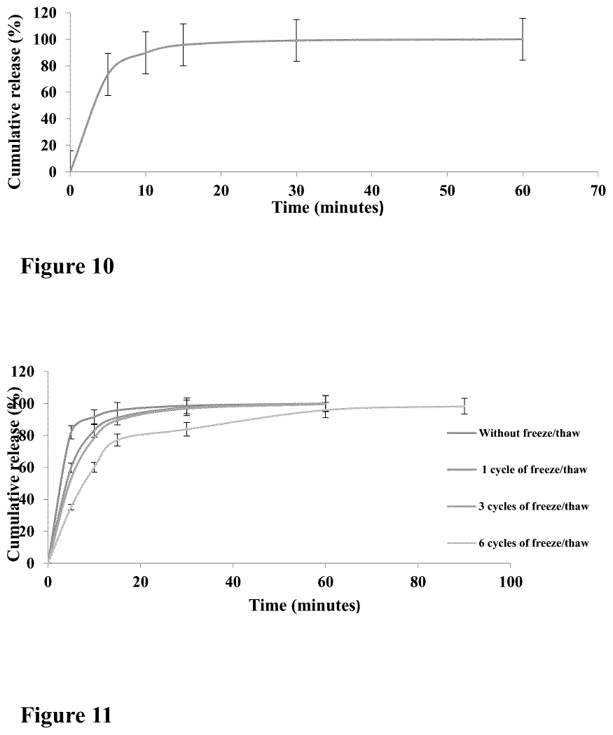

Release kinetics of bound H₂O₂ from PVA/PVP hydrogel to water was performed with PVA/PVP/H₂O₂ hydrogel with initial size of 1×1 cm², thickness of 0.25 cm and dry weight of 70.9 mg which was swollen with H₂O₂ aqueous solution up to final weight of 306.5 mg containing 8.6 w % entrapped H₂O₂. The above hydrogel was dipped in room temperature in vials containing 10 ml of DDW for different time periods, as described in the experimental part. Release kinetics of H₂O₂ from the hydrogel to the water is illustrated in FIG. 10.

The H₂O₂ percentage (w/w) released from the swollen hydrogen was about 73% within the first 5 min. and 100% (8.6%) within about 15 min. The hydrogel is capable of binding and diffusing the H₂O₂ solution from the surface throughout the 3D matrix of the hydrogels. The relatively slower H₂O₂ kinetics release from the hydrogel is due to the bound H₂O₂ molecules caged within the hydrogel 3D matrix. The matrix bound H₂O₂ molecules need to diffuse to the surface and only then can be released to the aqueous environment.

### Release Kinetics of H₂O₂ From PVA/PVP/UHP Hydrogel

Release kinetics of bound H₂O₂ from PVA/PVP/UHP hydrogel to water was performed with PVA/PVP/UHP hydrogel with initial size of 1×1 cm², thickness of 0.25 cm and wet weight of 117 mg containing 92.4 ml urea/H₂O₂ aqueous solution (7.8% H₂O₂ and 84.6% H₂O). Hereby, the PVA/PVP/UHP hydrogel was prepared in one step, excluding the water swelling step, while the H₂O₂ loaded PVA/PVP hydrogel was prepared in two steps: first the preparation of the PVA/PVP hydrogel followed by a water swelling step. This swelling process weakens the hydrogels 3D matrix strength²⁴ and, as a result, faster H₂O₂ Release kinetics may occur. The PVA/PVP/UHP hydrogel was dipped in room temperature in vials containing 10 ml of DDW for different time periods, as described in the experimental part. Release kinetics of H₂O₂ from the hydrogel to the water was then studied, as illustrated in FIG. 11.

The kinetic release tests were performed with hydrogels that underwent different freeze/thaw (FT) treatment cycles (1, 3 and 6 cycles). All samples demonstrated fast Release kinetics of H₂O₂ within the initial 15 min of the test after which the curve steadily increased until all H₂O₂ was released from all hydrogels (FIG. 11). This Figure demonstrates the effect of the number of freeze/thaw cycles on the Release kinetics of H₂O₂ to the water. Higher number of freeze/thaw cycles moderated and extend the release rate up to 90 min. As previously stated, the freeze/thaw process causes the formation of phase separation which may lead to a stronger 3D structure and longer H₂O₂ release rate and duration in the matrix.

C. Release Kinetics of H₂O₂ Vapor From PVA/PVP/UHP Hydrogels

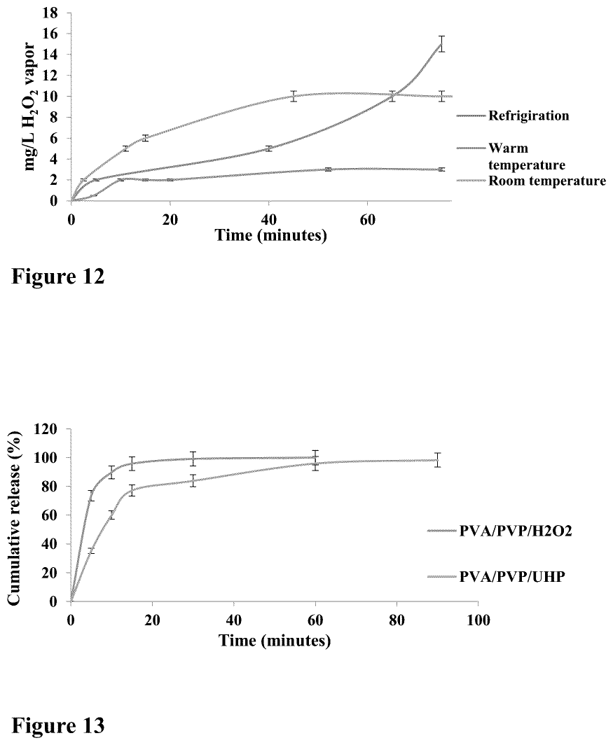

Release kinetics of H₂O₂ vapors from PVA/PVP/UHP hydrogel was performed as described in the experimental part. H₂O₂ vapors are much more sensitive to high temperatures than lower. With increasing temperature, H₂O₂ decomposes faster to water and oxygen, therefore the concentration reduces25 and evaporation accelerates26. Our trials indeed support this assumption. The sample stored in refrigeration, released, for the long run, the highest H₂O₂ concentration compared to the ones stored in room and warm temperatures, as shown in FIG. 12. Moreover, H₂O₂ vapor concentration equilibration time was depended on the temperature. The hydrogels which were stored in warm (30° C.) and room temperature reached H₂O₂ vapor concentration equilibration within 15 and 40 minutes, respectively. On the other hand, the hydrogel which was stored in cold (3-4° C.) its H₂O₂ vapors concentration did not reach the maximal concentration and equilibration time even after 75 min. Further work on this topic is ongoing in our lab.

### H₂O₂ Capacity and % Released to wWter of the PVA/PVP/H₂O₂ Hydrogel and PVA/PVP/UHP Hydrogel

Table 5 illustrates that the PVA/PVP/H₂O₂ hydrogel with initial size of 1×1 cm² and dry weight of 70.9 mg was swollen with H₂O₂ aqueous solution (H₂O/H₂O₂) up to final weight of 306.5 mg containing 8.6 w % H₂O₂ and PVA/PVP/UHP hydrogel with initial size of 1×1 cm² and weight of 117 mg containing 92.4 ml urea/H₂O₂ aqueous solution, 7.8% H₂O₂ and 84.6% H₂O. Thereby, under the experimental conditions the r H₂O₂ capacity of the PVA/PVP/H₂O₂ and PVA/PVP/UHP hydrogels is similar while the release kinetics is slightly slower for the PVA/PVP/UHP hydrogel as shown in FIG. 13.

TABLE 5
H₂O₂ capacity of the PVA/PVP/H₂O₂
hydrogel and PVA/PVP/UHP hydrogel

| [H₂O₂]/[(PVA/PVP | [H₂O₂]/[PVA/PVP/UHP] |
|---|---|
| H₂O₂)](w/w %) | (w/w %) |
| 8.6 ± 0.4 | 7.8 ± 0.4 |

### FTIR Characterization the PVA/PVP/H₂O₂ Hydrogel and PVA/PVP/UHP Hydrogel

### A. FTIR/ATR Spectrum of PVA/PVP Hydrogel

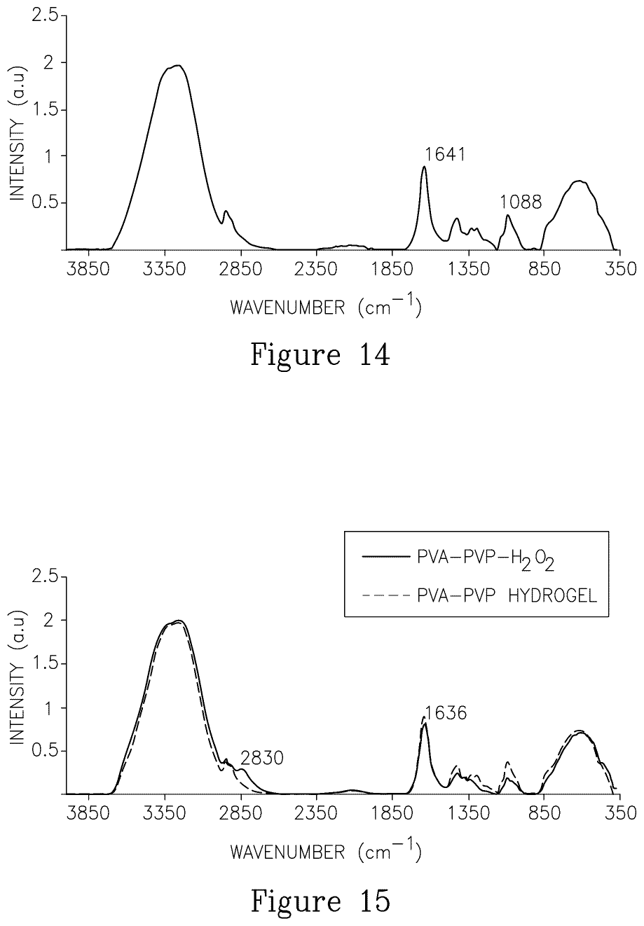

Experimental ATR analysis of PVA and PVP aqueous solutions resulted in C—O and carbonyl stretching bands at 1084 and 1645 cm⁻¹, respectively (FIG. 14).

B. FTIR/ATR Spectrum of PVA/PVP/H₂O₂ Hydrogel

PVA/PVP hydrogel, as described above, was swollen with hydrogen peroxide aqueous solution to produce PVA/PVP/H₂O₂ hydrogel (FIG. 15). As a consequence, the carbonyl assigned to PVP was shifted from 1641 c″m⁻1 to a lower frequency at 1636 cm⁻¹. This shift is probably due to the formations of PVP-H₂O₂ adducts¹⁶ where H₂O₂ is capable to form six possible hydrogen bond²⁸ with PVP in comparison to four possible hydrogen bonds with H₂O molecules²⁹. The appearance of the unique O—H stretch in 2830 cm-1 is assigned to H₂O₂ which confirms its presence in the hydrogel matrix.

### C. FTIR/ATR Spectrum of PVA/PVP/UHP Hydrogel

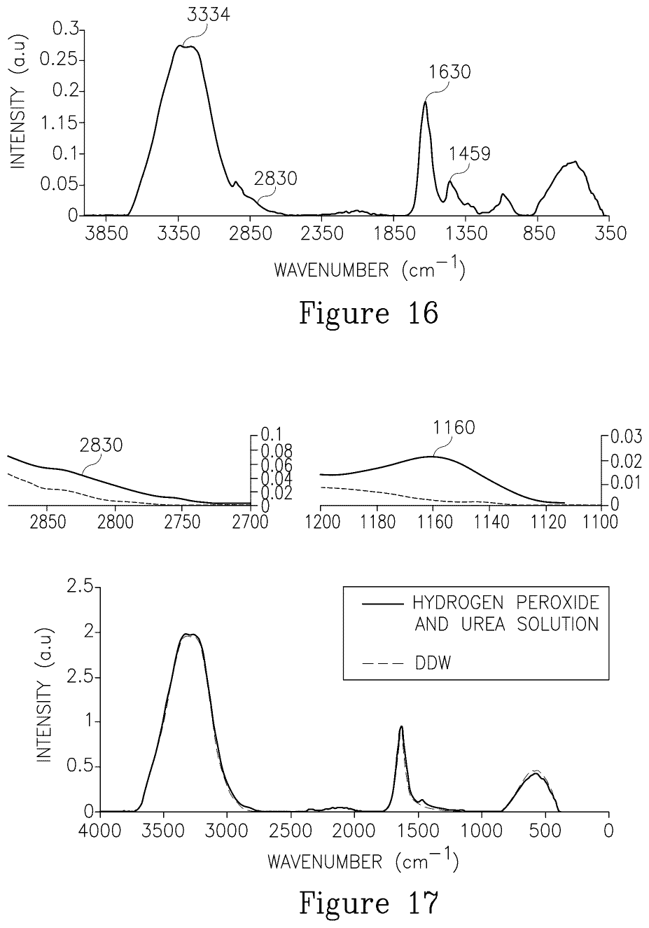

The preparation process of PVA/PVP/UHP hydrogels involves the in-situ addition of urea hydrogen peroxide (UHP) into the hydrogel, as previously described (FIG. 16). The addition of UHP may contribute to branched hydrogen bond interactions, since it is only capable of interacting with the two lone pairs electrons of the carbonyl group while donating the four hydrogens belonging to the primary amines (a total 6 bonds). As a consequence, the carbonyl stretching of PVP was shifted from 1641 c″m⁻¹ to a lower frequency at 1630 cm-⁻¹, a peak with a right shoulder. The sharp shift may have explained by dual contributors to the hydrogen bond caused by urea and hydrogen peroxide. The peak shoulder is due to the overlap of urea carbonyl stretching with the PVP carbonyl stretching. The shoulder at 2830 cm⁻¹, assigned to the O—H stretch of H₂O₂, confirms its appearance in the hydrogel matrix. 3334 cm⁻¹ and 1459 cm⁻¹ are N—H and C—N stretching assigned with urea, respectively.

D. FTIR/ATR Characterization of Urea and Hydrogen Peroxide Released From the PVA/PVP/UHP Hydrogel toWwater

When the hydrogels are dipped in water, as described previously, urea and hydrogen peroxide are released from the hydrogel to the aqueous phase environment. Their presence in the aqueous solution was determined by the detection of unique vibrations at 2830 and 1160 cm⁻¹ assigned to the O—H stretching of H₂O₂ and NH₂ rocking of urea, respectively (FIG. 17)³⁰. Both peaks were present in the solution and discernable in FIG. 17 when compared to the spectra of DDW.

### Light Microscope Surface Analysis of the PVA/PVP and PVA/PVP/UHP Hydrogels

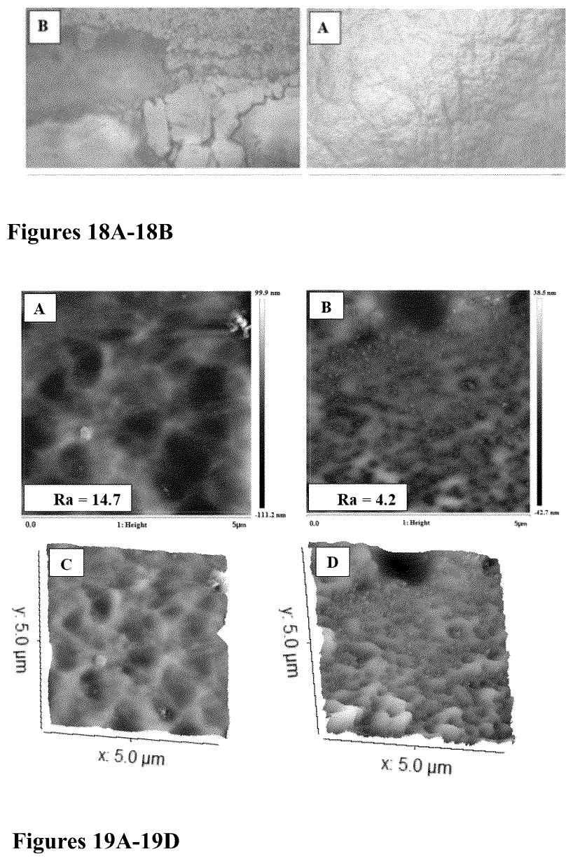

Surface analysis of the PVA/PVP and PVA/PVP/UHP hydrogels taken by our light microscope (magnification ×10) is demonstrated in FIG. 18. Morphological analysis of the hydrogels revealed significant different surface morphology. PVA/PVP hydrogel exhibited a homogenic coarse and amorphous morphology (FIG. 18A) while PVA/PVP/UHP showed heterogenic morphology of smooth, sharp and dotted textures (FIG. 18B). This difference morphology is probably due to the surface oxidation of the PVA/PVP by H₂O₂.

### Atomic Force Microscopy (AFM) Roughness Analysis the PVA/PVP and PVA/PVP/UHP Hydrogels

Surface morphology and roughness of the PVA/PVP and PVA/PVP/UHP hydrogels were studied with AFM using the Ra roughness average (FIG. 19). PVA/PVP (FIGS. 19A and C) exhibited a significantly higher roughness (14.7 nm) and clean texture compared to the lower roughness (4.2 nm) and dotted textures of the PVA/PVP/UHP hydrogel (FIGS. 19B and D). The dotted texture resembled those shown in the light microscope images of PVA/PVP/UHP (FIG. 18).

### Biological Activity of the PVA/PVP/UHP and PVA/PVP/H₂O₂ Hydrogels

### 4.8.1 pH Study

pH values are a significant environmental parameter in plant growth since the mobility of plant nutrients are directly influenced by it. The optimal pH range for plants is between 5.4-6.4³¹. The pH value of the hydrogel samples was 5.5, within the optimal pH range for plants. Two different biological tests were performed to assess the hydrogel activity on plants and pathogens. The hydrogels were tested either through direct contact with the plants and pathogens or through indirect contact where H₂O₂ was released from the hydrogel and transferred to the plant and pathogen through a liquid or gas state.

### 4.8.2 Phytotoxicity

### Phytotoxic Direct Test of PVA/PVP/UHP Hydrogel Patterned to Plant Nursery Seedling Plate

Broccoli seedlings were planted in the hydrogel plant nursery seedling plate (FIG. 5) and were grown for ˜3 weeks. The plant phytotoxic was monitored and logged during different time periods (FIG. 20). The seedlings in the left and right of each image were exposed to PVA/PVP/UHP and PVA/PVP hydrogel (control), respectively. Seedlings planted in the hydrogel plant nursery were compared to those untreated by hydrogel. PVA/PVP/UHP hydrogel treated seedlings exhibited: 1 day after planting, slight phytotoxic foliage signs were seen but no noticeable root system damage were discernable. Strong phytotoxic signs were detected after 2 days indicated by bleaching of the foliage, root system and soil. Images taken after 3 days of planting showed partial recovery indicated by a growth of young foliage. After 1 week, the broccoli seedlings deteriorated and showed severe signs of phytotoxic on the foliage, root system and soil (bleach effect). After 3 weeks, the seedlings had wilted.

During the test period, seedlings untreated by hydrogels exhibited no phytotoxic signs in the foliage and root system, as expected. Also, the second control group (PVA/PVP hydrogel) exhibited no phytotoxic signs in the foliage and root system. Slightly dehydration signs were noticed toward the final week of the test. Dehydration of the seedlings was caused by the dehydration of the hydrogel over time. When the hydrogel dehydrates, it absorbs water from the surrounding soil which deprives the plant from receiving a sufficient amount of water to continue growing. This was solved by an increase in watering regulations which led to the seedlings flourishing again. The PVA/PVP/UHP hydrogel caused bleaching of the soil and plants. This is due to the high concentration of H₂O₂ present in the hydrogel which reacted with the plant as well. This can be solved by adjusting the active material concentration and kinetics release to levels below the seedlings phytotoxic limit level.

Similar results to that of PVA/PVP/UHP hydrogel were observed for the PVA/PVP/H₂O₂ hydrogel.

### Phytotoxic Indirect Test of PVA/PVP/UHP Hydrogel Sheets

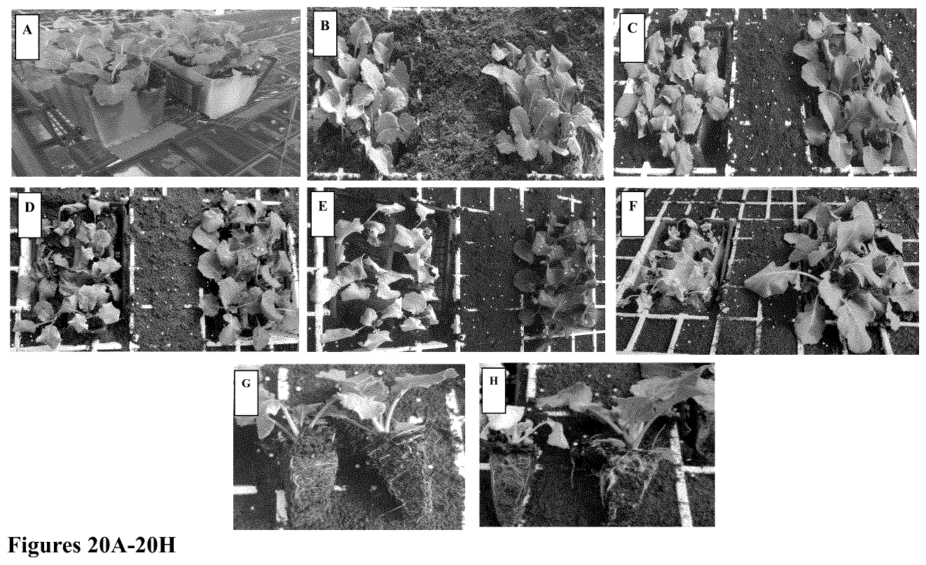

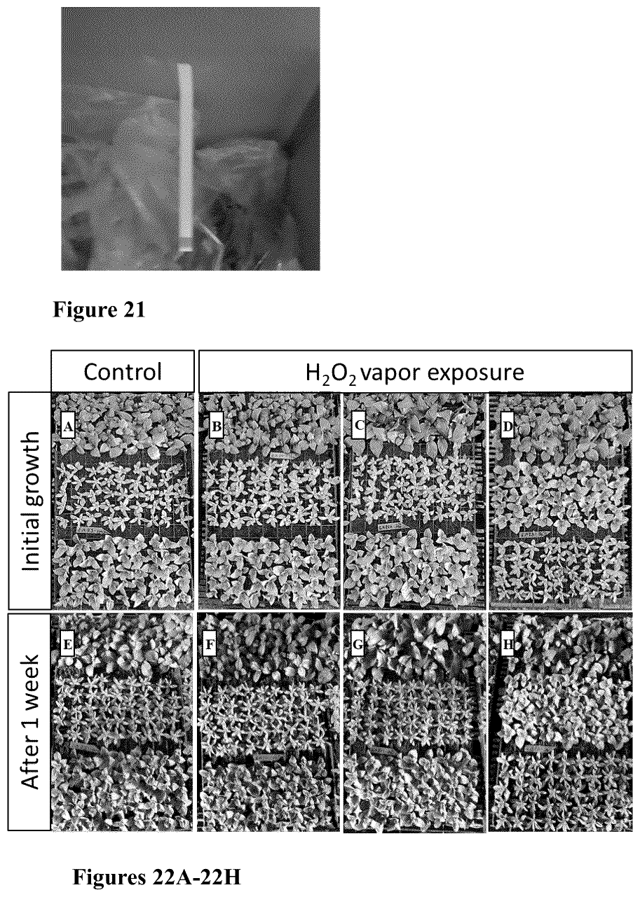

H₂O₂ vapor release hydrogel sheets placed in carton packages containing nylon wrapped plant cuttings and Quantofix peroxide strip (FIG. 21). The aim of this test was to determine if the H₂O₂ vapor released from the hydrogel sheets had phytotoxic effect on the wrapped cuttings. The carton package was opened following a one-day cooling period in the storage room to monitor the H₂O₂ vapor concentration (FIG. 6 B).

The strip color indicated H₂O₂ vapors concentration in the cuttings package were equivalent to ˜5-10 mg/L. About 48 h after cooling storage, the cuttings were planted and let grow for 2 weeks (FIG. 22). Both sets of cuttings exposed to PVA/PVP and PVA/PVP/UHP hydrogel sheets, grew completely without signs of phytotoxic effects.

Similar results to that of PVA/PVP/UHP hydrogel were observed for the PVA/PVP/H₂O₂ hydrogel.

### Activity Against Viruses

### Pesticide Effectiveness of PVA/PVP/UHP Hydrogel Against the Spread of Tomato Brown Rugose Fruit Virus (ToBRFV)

The test was conducted on tobacco and tomato plants. This was done through direct contact of the hydrogels with the virus and through indirect contact via diffusion of H₂O₂ from the hydrogels to the virus in their gas phase. The local lesions (LL) were counted on the tobacco plant leaves for the bioassay. The virus was exposed to the hydrogel via direct contact prior the bioassay (Table 6).

TABLE 6
ToBRFV bioassay of pre infected tobacco seedlings.
LLs were counted for each hydrogel sample

| ToBRFV- Bioassay No. | | Sample |
|---|---|---|
| local lesions (LL) | Sample type | number |
| >100 | PVA/PVP (control) | 1 |
| >100 | PVA/PVP (control) | 2 |
| 0 | PVA/PVP/UHP | 3 |
| 0 | PVA/PVP/UHP | 4 |
| 1 | PVA/PVP/UHP | 5 |
| 2 | PVA/PVP/UHP | 6 |
| 3 | PVA/PVP/UHP | 7 |

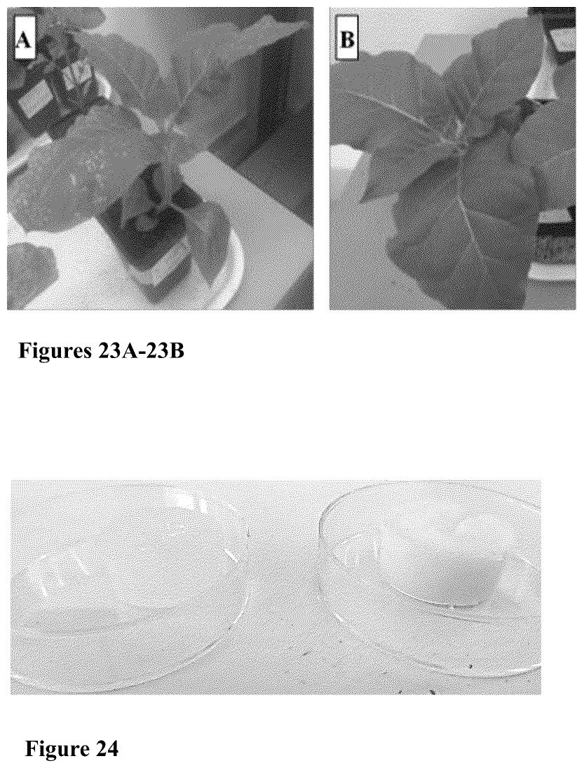

The results (Table 6 and FIG. 23) indicate almost complete killing effect of the virus exposed to the hydrogel containing UHP through direct contact, while PVA/PVP (control) did not affect the virus viability. The control tobacco seedling (FIG. 23A) exhibited expansive spreading of LLs. The seedling infected with the virus exposed to the PVA/PVP/UHP (FIG. 23B) exhibited healthy leaves with no signs of LLs. The LL bioassay for the tomato seedlings were inconclusive and could not be quantified visually. Therefore, ELISA serologic tests were applied instead (Table 7). The ELISA test exhibited ToBRFV infection in both control tomato seedlings infected with the virus exposed to the PVA/PVP hydrogel via direct and indirect modes. Therefore, the PVA/PVP hydrogel did not affect the virus viability. All infected seedlings infected with the virus directly exposed to the PVA/PVP/UHP hydrogel were negative to virus. The virus that was exposed indirectly (H₂O₂ vapors transferred via gas phase) to the PVA/PVP/UHP were also eliminated, except one repeat of the five. This confirms the PVA/PVP/UHP hydrogel was completely effective against the virus in the direct mode and was generally highly effective in the indirect mode.

TABLE 7
ELISA test results of ToBRFV in tomato seedlings

| Sample | | ToBRFV ELISA |
|---|---|---|
| number | Test mode | result |
| 1 | Indirect contact (control sample) | + |
| 2 | Indirect contact (control sample) | + |
| 3 | Direct contact (control sample) | + |
| 4 | Direct contact (control sample) | + |
| 5 | Indirect contact | + |
| 6 | Indirect contact | − |
| 7 | Indirect contact | − |
| 8 | Indirect contact | − |
| 9 | Indirect contact | − |
| 10 | Direct contact | − |
| 11 | Direct contact | − |
| 12 | Direct contact | − |
| 13 | Direct contact | − |
| 14 | Direct contact | − |

Similar results to that of PVA/PVP/UHP hydrogel were observed for the PVA/PVP/H₂O₂ hydrogel.

### In Situ Preparation of PVA/PVP/Thymol Hydrogels

Preparation procedure for 1% (W/W) thymol in PVA/PVP hydrogel: 15 gr of PVA were mixed in 79 gr of double distilled water at 80-90° C., until full dissolution. 6 gr of PVP were then added to the PVA solution until complete dissolution. The obtained solution was cooled down to 72 ºC, followed by addition of 61.5 mg of thymol. The obtained dispersion was stirred until homogeneous emulsion was obtained (about 5 min.). The suspension was then poured into the sample pattern and then placed in a freezer (−20° C.) for overnight. The casting was thawed in room temperature. The obtained PVA/PVP/thymol hydrogel was then sealed and stored in 4° C. Note: In addition, similar procedure, except the addition of thymol was done for the preparation of neat hydrogel (control), FIG. 24 illustrates that the PVA/PVP/thymol hydrogel is significantly more opaque than that of the PVA/PVP due to the encapsulated thymol.

### Detection of Thymol in PVA/PVP/Thymol Hydrogel by FTIR/ATR

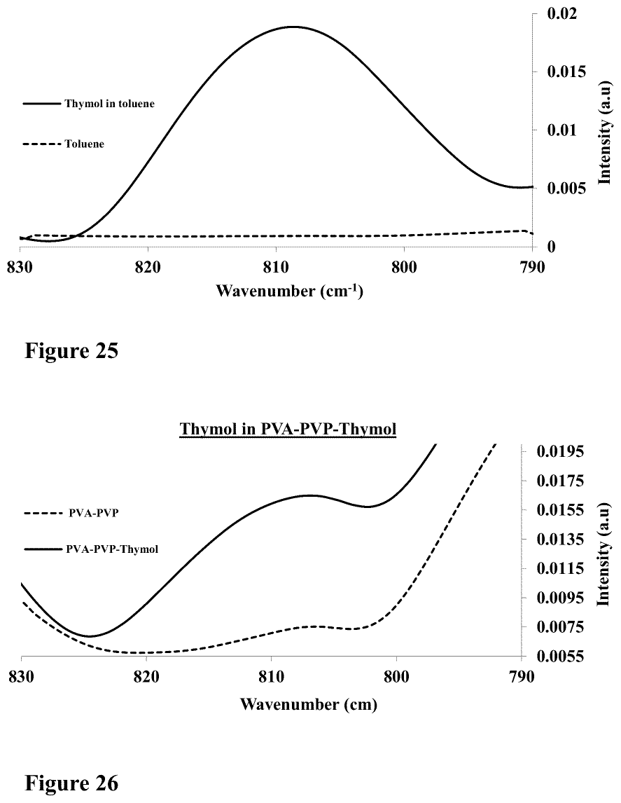

Pure thymol was detected by preparation of thymol (74 mg/ml) solution in toluene. The thymol solution was scanned against pure toluene solution and was differentiated on 809 cm⁻¹ C—H out of plane wagging vibration assigned with the thymol (FIG. 25). Direct scan of the PVA/PVP/thymol hydrogel was not sufficient for detection of the thymol unique signal at 809 cm⁻¹. Therefore, thymol was extracted from the hydrogel, by adding 851 mg of the hydrogel sample to100 μl of toluene. Similar procedure was done on control hydrogel (PVA/PVP hydrogel). FIG. 26 demonstrated the FTIR of the extracted thymol. The thymol extracted solution from the PVA/PVP/thymol hydrogel was scanned against extracted solution of the neat hydrogel. The appearance of 809 cm⁻¹ vibration in the PVA/PVP/thymol hydrogel confirmed the presence of thymol.

### In Situ Preparation of PVA/PVP/H₂O₂-Thymol Hydrogels

15 gr of PVA were mixed in 79 gr of double distilled water at 80-90° C., until full dissolution. 6 gr of PVP were then added to the PVA solution until complete dissolution. The obtained solution was cooled down to 60° C., followed by addition of the desired amount of thymol and H₂O₂. The obtained dispersion was stirred until homogeneous emulsion was obtained (about 5 min.). The suspension was then poured into the sample pattern and then placed in a freezer (−20° C.) for overnight. The casting was thawed in room temperature. The obtained PVA/PVP/H₂O₂-thymol hydrogels were then sealed and stored in 4° C.

### Activity Against Pests (Insects)

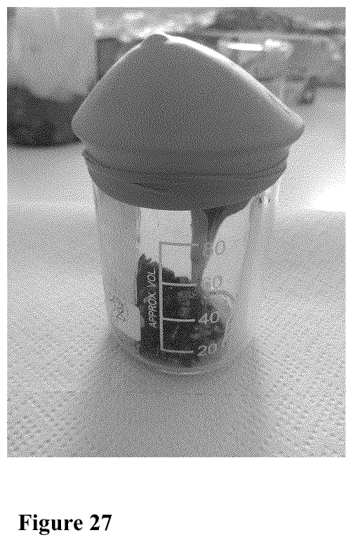

For the present experiment, small plants of cucumbers and zucchini were placed in a beaker (FIG. 27). Insects such as silverleaf whitefly (Bemisia tabaci), a leaf aphid, and pseudococcidae, a floury aphid, were placed on the plant leaves. PVA/PVP, PVA/PVP/thymol(10%), PVA/PVP/H₂O₂(10%) and PVA/PVP/H₂O₂(5%)-thymol(5%) hydrogel sheets were attached to the wall of the beakers without any contact with the leaves (FIG. 17). The beakers were hermetically closed and placed in refrigeration)(6° C. After 12 hours the beakers were placed in room temperature for 3 hours after which the viability of the insects were tested and determined. The results of this experiment clearly show that no harm was done to the insects by the PVA/PVP control hydrogel. On the other hand, both the PVA/PVP/thymol and PVA/PVP/H₂O₂ hydrogel demonstrated 90-100% killing of the leaf aphids but hardly damaged the floury aphids, while the PVA/PVP/H₂O₂-thymol hydrogel illustrated 100% killing of both the leaf and the floury aphids. These results indicate a clear synergism effect between H₂O₂ and the thymol oil.

### Preparation of Additional PVA/PVP Hydrogels

### In Situ Preparation of PVA/PVP/Pheromone, PVA/PVP/Pesticide and PVA/PVP/BP Hydrogels

Encapsulation of dodecyl alcohol (a pheromone model), fluazinam (a pesticide model), and benzoyl peroxide (BP) in the PVA/PVP hydrogel for medical/cosmetic/agriculture applications were done similarly substituting the thymol for dodecyl alcohol, or fluazinam, or BP.

### In Situ Preparation of PVA/PVP/TCA Hydrogel

### Preparation of 10% TCA in PVA-PVP Hydrogel

7.5 gr of PVA was mixed in 39.75 gr of double distilled water (DDW) at 90-95° C., until full dissolution. 3 gr of PVP was added to the solution until complete dissolution. Then, the PVA-PVP solution was let cool down to 65° C. 4.0 gr TCA was poured into the solution and mixed until homogeneous white emulsion was observed. The emulsion was mold into 50 mm petri dishes and were cooled down to room temperature. The castings were placed in freezer (−20° C.) for overnight furthered thaw (freeze-thaw cycle). The freeze-thaw procedure was repeated additional four cycles (total five freeze-thaw cycles), resulted in plate shape PVA/PVP/TCA hydrogels. The hydrogels were sealed in plastic with aluminum inner coating hermetic zip bags (13×13 cm²). Similar procedure, except for the addition of TCA, was done for the preparation of PVA-PVP hydrogel control samples.

### PVA-PVP-TCA Hydrogels Stability Test

The durability and samples integrity overtime was tested. The presence of TCA monitoring by pH test strips (MACHEREY-NAGEL, PH-Fix 0-14). The hydrogels integrity performed by visual and contact inspection. The hydrogels were tested in various storage temperatures: 23-25° C., −20° C., 2-4° C. PVA/PVP control hydrogels were monitored in a similar way.

### Results

PVA/PVP/TCA and PVA/PVP hydrogels initial (time zero-hydrogels after preparation) pH measurements indicated values between 0-1 and 6-7, respectively. All hydrogels integrity was confirmed, after the initial measurements. Each set of samples (hydrogel with and without TCA) were stored in different temperatures as was described above. pH measurements and integrity inspections were repeated after a month. The hydrogels pH values and integrity remained unchanged.

### Preparation of PVA/PVP/MO, PVA/PVP/CB and PVA/PVP/Mⁿ⁺ Hydrogels by a Swelling Method

PVA/PVP/MO (Methyl orange) hydrogel was prepared by dissolving 7.5 gr of PVA in 39.5 gr of DDW in a 100 ml Erlenmeyer flask. The flask was placed in a silicon oil bath at 90-95° C. until complete dissolution. 3 gr of PVP was added to the PVA solution and was left to react for 1.5 hours. The solution was then cooled to 70° C. (until the foam from the reaction disappeared). The solution was then poured into a desired mold. The obtained molded hydrogel was placed in a freezer (approximately −18° C.) overnight, then thawed (freeze/thaw cycle). The number of freeze/thaw cycles varied depending on the desired properties. The hydrogel was then stored in 4° C. Swelling of the PVA/PVP with H₂O containing MO was then accomplished by dipping the PVA/PVP hydrogel in a flask containing 0.5% MO aqueous solution (10 mL) for a few hours. All the aqueous solution was swollen by the PVA/PVP red hydrogel.

Similar results were obtained by substituting the MO aqueous solution for Cibacron blue (CB) dye aqueous solution (0.2%). Similar results were obtained by substituting the MO aqueous solution for metal ions aqueous solutions, e.g., Cu⁺².

### In Situ Preparation of PVA/PVP/MO, PVA/PVP/CB and PVA/PVP/Mⁿ⁺ Hydrogels

The PVA/PVP hydrogel was prepared according to FIG. 3, by dissolving 7.5 gr of PVA in 39.5 gr of DDW in a 100 ml Erlenmeyer flask. The flask was placed in a silicon oil bath at 80-95° C. until complete dissolution. 3 gr of PVP was added to the PVA solution and reacted for 1.5 hours. The solution was cooled to 70-78° C., then appropriate concentration of MO, or CB or metal ion e.g. Cu²⁺ was added to the Erlenmeyer flask until complete dissolution. The obtained molded hydrogel was then immediately poured into a desired mold. The obtained PVA/PVP/MO or PVA/PVP/CB or PVA/PVP/Mⁿ⁺ mold was then placed in a freezer (approximately −18° C.) overnight and then thawed (freeze/thaw cycle). The number of freeze/thaw cycles varied depending on the desired hydrogel properties. The hydrogel was then stored in 4° C. If necessary, covalent cross-linking of the hydrogels will be accomplished with glutaraldehyde through the PVA.

### Synthesis of Biodegradable PVA/PVP Polymeric Films

PVA/PVP hydrogels were prepared by heating PVA aqueous solution at approximately 95° C. until dissolution, followed by addition of PVP and subsequent cooling down to room temperature. Briefly, 7.5 gr of PVA were dissolved in 39.5 gr double-distilled water (DDW) at approximately 95° C. 3 gr of PVP were then added to the PVA solution and stirred for 1.5 h. The solution was then cooled to approximately 78 0 C (until the foam from the reaction disappeared) and poured into corona pretreated plastic sheet. The hydrogels were spread by Mayer rod technique, with 100, 200, 400 and 1000 μm blades. The hydrogel coated sheets were dried overnight at room temperature or freeze at −180 C followed with thaw. The selected Mayer rod blade size varied, depending on the desired hydrogel strength properties. The hydrogel film strength increased as the layer was thicker. Dried hydrogel obtained transparent sheet while the freeze-thaw films had opaque appearance.

### Example 2

### Materials and Methods

### Fabrication of Non-Loaded, HP-Loaded, Thymol-Loaded, HP and Thymol-Loaded PVA/PVP Hydrogel Solutions

PVA/PVP hydrogel was prepared by heating PVA aqueous solution at 95° C. until dissolution, followed by addition of PVP and subsequent cooling down to room temperature. Briefly, 3.75 gr of PVA (average M.W 89,000-98,000, 98% hydrolyzed, Sigma-Aldrich) were dissolved in 19.75 gr double-distilled water (DDW) at 95° C. 1.5 gr of PVP K-30 (average M.W 40,000-80,000 from ISP) were then added to the PVA solution and stirred for 1.5 h. The solution was then cooled to 78° C.(until the foam from the reaction disappeared). HP, thymol or HP and thymol-loaded PVA/PVP hydrogel solutions were prepared by addition of urea-hydrogen peroxide (97% purity, Alfa Aesar), or HP, and thymol (>98.5% purity, SIGMA) into the PVA/PVP hot preheated (78° C.) solution and short mixing until complete dissolution. HP and thymol content were varied depend on the desirable concentrations.

### Hydrogel Coatings and Gelation Process

The hydrogel coatings were prepared on polyethylene (PE) films. The polyethylene surfaces were first treated with corona (350 W·min/m2, 20 scans) for surface activation. Immediate after mixing the hydrogel solutions as described in chapter 2.2.1. The hot hydrogel solutions (78° C. ) then poured into the corona pretreated surfaces, followed by Mayer Rod coating method with blade sizes of 100,200 and 400 microns). The hydrogel coated surfaces gelation was done by the freezing-thawing method as follow. The coated films were refrigerated overnight at −18° C. then thawed (1 freezing-thawing cycle). Another possibility is to crosslinked the hydrogels with glutaraldehyde or Boric acid.

### Molecular Characterization of Hydrogel cCatings

Attenuated Total Reflectance Fourier Transform infrared (ATR-FTIR) spectra of thymol, HP, thymol and HP loaded and unloaded PVA/PVP hydrogels, were characterized at room temperature by using Bruker ALPHA-FTIR Quicksnap™ sampling module equipped with Platinum ATR diamond module (Bruker, Germany) and translated by the OPUS program. The absorbance measurements were conducted in the range of 500-4000 cm⁻¹.

### Determination of Thymol Content in Thymol, Thymol and HP-Loaded PVA/PVP Hydrogels

Hydrogel coatings with size of 4 cm², were peeled from the polyethylene coated surface. The peeled hydrogels were centrifuged (6000 rpm, 30 minutes) in tube with 20 ml distilled water, until complete thymol extraction. Thymol aqueous solution absorption of pre-calibrate curve (λ=273 nm max absorption) was carried out with CARY 1E uv-visible spectrophotometer varian. The hydrogel extracted thymol concentrations were calculated from the thymol calibration curve equation. Measuring thymol contents were done in triplicates. Each test was done in duplicate. Determination of hydrogen peroxide (HP) content in HP-loaded PVA/PVP hydrogel coating.

HP loaded hydrogel coatings with size of 4 cm², were peeled from the polyethylene coated surface. The peeled hydrogels were dipped in vials containing 10 ml of DDW and were shaken at room temperature until complete HP extraction into the water. The concentration of the released HP was then determined by adding 550 μl of H₂SO₄ (96%) to the vial and titrating with a pre-calibrated KMnO₄ solution. Measuring HP contents were done in triplicates. Determination of hydrogen peroxide content in HP and thymol-loaded PVA/PVP hydrogel coating.

Hydrogel coatings with size of 4 cm², were peeled from the polyethylene coated surface. The peeled hydrogels were centrifuged (6000 rpm, 30 minutes) in tube with 20 ml distilled water, until complete HP extraction. The extracted HP released from the hydrogels was quantified by peroxide test sticks (Mancherey-Nagel Quantofix™). The test sticks are semi-quantitative visual colorimetric indicators with a wide peroxide measuring range (1-100 mg/L). The indicator sticks are pre-calibrated and accurate at a pH range of 2-9 and a temperature range of 4-30° C. The observed color is compared to the color scale bar. Measuring HP contents were done in triplicates.

### Release Rate Tests of Thymol and Hydrogen Peroxide

Petri dishes were filled with 3 g hay further sprayed with 4 g water. In order to prevent direct contact between the hydrogel coatings and the hay, the prefilled petri dishes were wrapped with plastic net. The hydrogel coated PE films were cut to the size and shape of petri dish (100 mm). The pre-cut coated films were placed on the net with the hydrogel coating oriented to the hay direction, while the PE film side is in contact with the petri dish cover. The petri dishes were incubated in 27° C., 55% humidity.

### Release Rate of Thymol

Thymol, HP and thymol loaded PVA/PVP hydrogel coatings with size of 4 cm², were peeled from the polyethylene coated surface. The peeled hydrogels were centrifuged (6000 rpm, 30 minutes) in tube with 20 ml distilled water, until complete thymol extraction. Thymol aqueous solution absorptions (λ=273 nm max absorption) were carried out with CARY 1E uv-visible spectrophotometer varian. Each absorption representing the remain thymol concentration relative to the time after the initial count, as follows: 2, 5 and 12 days The hydrogel extracted thymol concentrations were calculated from the thymol calibration curve equation. Measuring thymol release rates were done in triplicates.

### Release Rate Test of Hydrogen Peroxide

HP and thymol loaded PVA/PVP hydrogel coatings. HP and thymol loaded PVA/PVP hydrogel coatings with size of 4 cm², were peeled from the polyethylene coated surface. The peeled hydrogels were centrifuged (6000 rpm, 30 minutes) in tube with 20 ml distilled water, until complete HP extraction. HP concentration in the aqueous solution, were determined by insert the peroxide test sticks (Mancherey-Nagel Quantofix™) into the HP extraction solutions and compare the obtained stick color to color scale bar. Each sampling representing the remain HP concentration relative to the time after to the initial count, as follows: 2, 5 and 12 days. Measuring HP release rates were done in triplicates.

### HP Loaded PVA/PVP Hydrogel Coatings

HP loaded hydrogel coatings with size of 4 cm², were peeled from the polyethylene coated surface. The peeled hydrogels were dipped in vials containing 10 ml of DDW and were shaken at room temperature until complete HP extraction into the water. The concentration of the released HP was then determined by adding 550 μl of H₂SO₄ (96%) to the vial and titrating with a pre-calibrated KMnO₄ solution. Each sampling representing the remain HP concentration relative to the time after to the initial count, as follows: 2, 5 and 12 days. Measuring HP contents were done in triplicates.

### Morphological Characterization

The effect of the entrapped thymol, HP and thymol combined with HP on the surface morphology of the hydrogel coatings were revealed using a scanning electron microscopy (SEM) with magnifications of 200, 2,000 and 10,000. Morphological characterization was conducted under low vacuum pressure, 9.5 mm work distance and under voltage of 5 kV. The entrapped thymol particles and the holes formed in the HP loaded sizes, were calculated by image J program.

### Thermal Stability

Thermal gravimetric analysis (TGA) was carried out using SETSYS, by Setaram. The released products during the measurements were obtained by using Hiden HPR-20 QIC mass spectrometer (TGA) coupled with mass spectroscopy (MS). The analysis was conducted to study the stability effect of the PVA/PVP hydrogel, under heat treatment, on thymol, entrapped in the hydrogel, relative to thymol pure form. The tested samples were heated from 30° C. up to 630° C. (20° C./min heat rate) under nitrogen inert atmosphere (20 ml/min nitrogen flow rate).

### Results and Discussion

### FTIR (ATR) Spectra of PVA/PVP Hydrogels Entrapped With HP, Thymol

FTIR (ATR) spectroscopy was used to assess the chemical composition differences between neat PVA/PVP and thymol or HP loaded hydrogels. PVA/PVP neat and HP entrapped FTIR (ATR) characteristics were profoundly investigated in previous works³². The addition of thymol to the PVA/PVP major contribution is hydrogen bond interaction , since the thymol phenolic hydroxyl group (O—H stretching in 3269 cm⁻¹) capable of interacting with the PVP carbonyl group or the PVA hydroxyl group. Thymol molecular structure analysis by FTIR (ATR) is well known. However, when the thymol was entrapped in the PVA/PVP hydrogel, the thymol characterized vibrations were shifted to higher wavenumber. The entrapped thymol vibrations 846 cm^{-1,} 1091 cm⁻¹, 1427 cm⁻¹, 1653 cm⁻¹, 2919 cm⁻¹ and 2944 cm⁻¹ are assigned with C—H waging out of plane, 1:3:4-substitution, isopropyl group symmetric and asymmetric bending, C═C stretching, C—H symmetric and asymmetric stretching, respectively. The sharp shift of thymol may be explained by the interaction of thymol with the hydrogel. The observed thymol vibrations confirmed its presence in the PVA/PVP hydrogel matrix.

HP, Thymol or HP Combined with Thymol Contents in the Hydrogels

The measured HP content in the HP entrapped PVA/PVP/ hydrogel (5% w/w HP precursor) was 15.5±0.61 (w %). The measured thymol content in the thymol entrapped PVA/PVP hydrogels (5% and 1.25% w/w thymol precursor) was 19.6±1.32 and 2.5±0.51 respectively. The actual HP and thymol contents in the coatings are more concentrated than in its precursors.

HP release rate from HP, thymol and HP loaded PVA/PVP hydrogel coatings.

The HP release rates were tested on HP loaded hydrogel coating samples with size and weight of 4 cm² and 25 mg respectively. The HP average content (w %) was 15.5±0.61. The hydrogel coatings demonstrated fast release rates of HP within the first 2 days of the test, after which the curve moderately increased until all HP was released from all hydrogels after 8 days Additional HP release rates were measured on HP and thymol loaded hydrogel coatings. The coatings contained varied contents of thymol and HP. The coatings size and average weight is 4 cm² and 80 mg respectively. The thymol and HP loaded hydrogel coatings with varied HP and thymol contents, present similar HP release rates. The hydrogel coatings demonstrated fast release rates of HP within the first 2 days of the test, after which the curve moderately increased until all HP was released from all hydrogels after 3 days. Loading the hydrogels with varied HP concentrations did not affect much the HP release rate. HP loaded hydrogels demonstrated moderated and extended HP release rate in relative to those which were loaded with HP and thymol. The addition of thymol accelerated the HP release rate.

Thymol release rates from thymol, thymol and HP loaded PVA/PVP hydrogel coatings.

The thymol release rates were tested on thymol (5% and 1.25% w thymol precursor) loaded PVA/PVP hydrogel coating samples with size and average weights of 4 m² and 37±58.72.5±13 mg respectively. The measured thymol content in the thymol entrapped PVA/PVP hydrogels (5% and 1.25% w/w thymol precursor) was 19.6±1.32 and 2.5±0.51 respectively. Both hydrogel coatings demonstrated fast release rates of thymol within the first 2 days of the test, while the 5% w thymol precursor coating released less thymol (˜70% thymol cumulative release) than the 1.25% w thymol precursor coating (˜90% thymol cumulative release). After which the 5% w thymol precursor coating curve moderately decreased until the fifth day followed with mild release rate up to the 12 day, while the 1.25% w thymol precursor coating curve moderately decreased until the ninth day, followed with increase than decrease release rates during the executive 2 days, respectively. Loading heavier thymol concentration result milder release rate.

PVA/PVP coatings loaded with low HP and thymol contents (0.625% HP and 0.625% thymol precursors), exhibited fast thymol release rate and complete release occurred within 2 days from the test beginning. However, the coatings loaded with the medium (1.25% HP and 1.25% thymol) and high contents (2.5%, HP and 2.5% thymol precursors respectively) show more moderate thymol release rate. The high and medium HP and thymol content coatings released about 95% of the thymol within 4 days from the test beginning followed with mild thymol reload into the coatings (˜10%), during the rest 8 days of the test. The loaded Coatings with high HP and thymol contents observed slightly milder release rate and higher total thymol release (%) in relative to the medium HP and thymol contents coatings. The thymol mid and high loaded concentrations do not affect much the release rate, while the low concentration loaded released the entire thymol earlier and without reload itself. Thymol loaded hydrogel coatings (5% thymol precursor) introduce fast thymol linear release rate during the 2 days followed with moderated rate until the 2.5 days from the test beginning with maxi thymol loss of more than 70%. The followed step introduced thymol reload (˜15%) into the hydrogel coating until the fifth day, furthered with mild thymol release until the test end (12 days). HP and thymol loaded hydrogel coatings (2.5% HP and 2.5% thymol precursor) introduce fast thymol linear release rate during the 2 days followed with moderated convex rate until the fifth day with maximal 95% thymol loss from the test beginning. The followed step introduced thymol reload (˜15%) into the hydrogel coating until the test end. The HP presence mainly decreased the % w of thymol released from the coating.

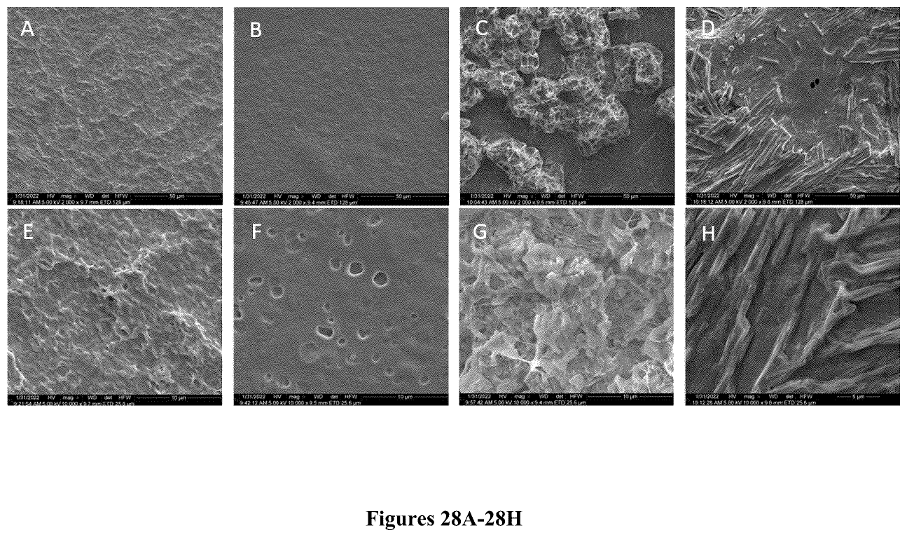

SEM images of the exemplary PVA/PVP hydrogel coating with or without HP/thymol are presented in FIG. 28. PVA/PVP hydrogel neat coatings introduce rough surface, while the HP loaded ones have smooth texture with average size of 1.27±0.44μ round shape holes. The SEM images verify the presence of thymol particles entrapped in the PVA/PVP hydrogel coatings. The thymol average particle size was 146±57μ. The entrapped thymol particles introduce web and hollow with inner discs and flakes shapes. The HP and thymol loaded hydrogel has smooth texture with dense net fiber shape. Inner view exposes continuous rods shape with average width of 1.43±0.23μ. HP, thymol, HP and thymol form different coating morphologies.

The PVA/PVP hydrogel coating effect on the thymol thermal stability as measured by TGA-MS

All the pure thymol loss occurred during one step, starting from 2 (70° C.) and ended in 5 (130° C.) minutes. The continuous thymol reading after 5 minutes, is a result of retained thymol residues on the MS detector. The thymol entrapped in the PVA/PVP hydrogel loss was in one step begins from 22 (470° C.) and end in 25 (530° C.) minutes. The HP and thymol entrapped in the PVA/PVP hydrogel, thymol loss occurred in one step begin from 21.5 (460° C.) and end in 25 (530° C.) minutes.

### HP, Thymol, HP and Thymol PVA/PVP Hydrogel Coatings Anti-Mold Activity

The test was done on petri dishes. The coated poly ethylene (PE) sheets were located on the petri dish top and the hay was placed on the bottom. Spacer nets were placed beneath the coated PE sheets and the hay. The mold growth exposed to the various sheets, was monitored. Un-coated PE show complete mold development within 12 days. Thymol, HP and thymol loaded PVA/PVP hydrogel coated PE sheets prevent any mold growth, while the HP loaded coating introduce temporary partial inhibition followed with fully mold growth. Complementary test was conducted, in order to identify minimal mold inhibition concentration of thymol, HP and thymol. HP and thymol (0.625% w of each) enable mold development (30%) within 6 days from the test beginning. The exposed mold to the thymol (1.25% w) loaded coating, completely eliminated mold development. Therefore, the coatings loaded with thymol only, were found as more effective than those loaded with HP and thymol. The thymol minimal mold inhibition concentration was furthered tested. Thymol (0.625% w) loaded coating introduce mold growth (10%) after 11 days from the test beginning.

The hay which was exposed to the non- coated PE (control) sheet, introduced fully developed mold within 8 days, while all the HP and thymol loaded hydrogel coated PE treatments, prevent any mold growth, even with each low as 1.25% w HP and thymol.

### Example 3

### Preparation of PVA/PVP Hydrogel Beads

PVA/PVP hydrogel was prepared by dissolving 10 gr of PVA in 100 gr of DDW and stirred for 1h at 95° C. to form homogenous solution. 4 gr of PVP was added to the PVA solution and was left at 95° C.until complete dissolution. The solution was then cooled to 25° C., which followed by the addition of 5% NaOH solution until pH was 10. Then, alkali PVA/PVP hydrogel was added dropwise to cross-linked solution via syringe (cross-linked solution containing 4% of Boric acid and 3% of CaCl₂) and spherical beads were formed. The spherical PVA/PVP beads were covalent and cross-linking for different time periods (5 min, 10 min, 15 min, 30 min, and 60 min). The gel beads were taken out carefully and washed with deionized water till pH was neutral. The obtained PVA/PVP hydrogel beads were stored in a refrigerator in 4° C. The diameter of the PVA/PVP beads could be controlled (0.2 mm up to ca. 15 mm) by changing conditions such as the ratio of PVA/PVP, needle size and type (glass or plastic such as teflone), etc.

### Preparation of PVA/PVP/Thymol Hydrogel Beads

PVA/PVP hydrogel was prepared by dissolving 10 gr of PVA in 100 gr of DDW and stirred for 1h at 95° C. to form homogenous solution. 4 gr of PVP was added to the PVA solution and was left at 95° C. until complete dissolution. The solution was then cooled to 60° C., which followed by the addition of Thymol that dissolved 5% NaOH solution. Then, alkali PVA/PVP/Thymol hydrogel was added dropwise to cross-linked solution via syringe (cross-linked solution containing 4% of Boric acid and 3% of CaCl₂) and spherical beads were formed. The spherical PVA/PVP/Thymol beads were covalent and cross-linking for different time periods (5 min, 10 min, 15 min, 30 min, and 60 min). The gel beads were taken out carefully and washed with deionized water till pH was neutral. The obtained PVA/PVP/Thymol hydrogel beads were stored in a refrigerator in 4° C.

### Preparation of PVA/PVP/SA Hydrogel B eads

PVA/PVP/SA hydrogel was prepared by dissolving 1 gr of Sodium Alginate in 100 gr of DDW and stirred at 95° C. to form homogenous solution. Then 10 gr of PVA was added to SA solution until PVA dissolved completely. 4 gr of PVP was added to the PVA/SA solution and was left at 95° C. until complete dissolution. The solution was then cooled to 25° C., which followed by the addition of 5% NaOH solution. Then, alkali PVA/PVP/SA hydrogel was added dropwise to cross-linked solution via syringe (cross-linked solution containing 4% of Boric acid and 3% of CaCl₂) and spherical beads were formed. The spherical PVA/PVP/SA beads were covalent and physically and cross-linking for different time periods (5 min, 10 min, 15 min, 30 min, and 60 min). The gel beads were taken out carefully and washed with deionized water till pH was neutral. The obtained PVA/PVP/SA hydrogel beads were stored in a refrigerator in 4° C.

### Preparation of PVA/PVP/SA/UHP Hydrogel Beads

PVA/PVP/SA hydrogel was prepared by dissolving 1 gr of Sodium Alginate in 100 gr of DDW and stirred at 95° C. to form homogenous solution. Then 10 gr of PVA was added to SA solution until PVA dissolved completely. Then 4 gr of PVP was added to the PVA/SA solution and was left at 95° C. until all ingredients dissolve completely to homogenous hydrogel. The hydrogel was then cooled to 25-30° C., which followed by the addition of UHP. Then, PVA/PVP/SA/UHP hydrogel was added dropwise to cross-linked solution via syringe (cross-linked solution containing 4% of Boric acid and 3% of CaCl₂) and spherical beads were formed. The spherical PVA/PVP/SA/UHP beads were covalent physically and cross-linking for different time periods (5 min, 10 min, 15 min, 30 min, and 60 min). The gel beads were taken out carefully and washed with deionized water till pH was neutral. The obtained PVA/PVP/SA/UHP hydrogel beads were stored in a refrigerator in 4° C.

### Preparation of PVA/PVP/SA/Thymol/UHP Hydrogel Beads

PVA/PVP/SA hydrogel was prepared by dissolving 1 gr of Sodium Alginate in 100 gr of DDW and stirred at 95° C. to form homogenous solution. Then 10 gr of PVA was added to SA solution until PVA dissolved completely. Then 4 gr of PVP was added to the PVA/SA solution and was left at 95° C. until all ingredients dissolve completely to homogenous hydrogel. The solution was then cooled to 50-60° C., which followed by the addition of Thymol and UHP. Then, PVA/PVP/SA hydrogel was added dropwise to cross-linked solution via syringe (cross-linked solution containing 4% of Boric acid and 3% of CaCl₂) and spherical beads were formed. The spherical PVA/PVP/SA/Thymol/UHP beads were covalent and physically cross-linking for different time periods (5 min, 10 min, 15 min, 30 min, and 60 min). The gel beads were taken out carefully and washed with deionized water till pH was neutral. The obtained PVA/PVP/SA/Thymol/UHP hydrogel beads were stored in a refrigerator in 4° C.

### Preparation of PVA/PVP/SA/Fluazinam Hydrogel Beads

PVA/PVP/SA hydrogel was prepared by dissolving 1 gr of Sodium Alginate in 100 gr of DDW and stirred at 95° C. to form homogenous solution. Then 10 gr of PVA was added to SA solution until PVA dissolved completely. Then 4 gr of PVP was added to the PVA/SA solution and was left at 95° C. until all ingredients dissolve completely to homogenous hydrogel. The hydrogel was then cooled to 25-30° C., which followed by the addition of Fluazinam. Then, PVA/PVP/SA/Fluazinam hydrogel was added dropwise to cross-linked solution via syringe (cross-linked solution containing 4% of Boric acid and 3% of CaCl₂) and spherical beads were formed. The spherical PVA/PVP/SA/Fluazinam beads were covalent physically and cross-linking for different time periods (5 min, 10 min, 15 min, 30 min, and 60 min). The gel beads were taken out carefully and washed with deionized water till pH was neutral. The obtained PVA/PVP/SA/Fluazinam hydrogel beads were stored in a refrigerator in 4° C.

### Phytotoxicity Tests

### Hydrogel Beads Phytotoxicity Indirect Test on Banana Clusters

200 gr of PVA/PVP/Thymol and PVA/PVP/Thymol/UHP hydrogel beads were prepared like described in section . For all hydrogel combinations were done 3 replicates. Every example anchored in the middle of banana clusters. The banana clusters were monitored twice by visual inspection for phytotoxicity. The test lasted for three months. Phytotoxicity range was determined between 0-5 levels.

### PVA/PVP/Thymol Emulsions Phytotoxicity Direct Test

The emulsions were sprayed on different varieties of seedlings. Phytotoxicity monitoring was done by visual inspection during the 48-72 h from the test beginning. The treated seedlings demonstrated wide range of phytotoxicity effects, started from none or minor signs to more dominant damages.

### Example 4

### PVA/PVP/HP/THYMOL Hydrogel Capsule Preparation

The PVA/PVP hydrogel was, by dissolving 26.6 gr of PVA in 140 gr of distilled water. The PVA solution was placed in a silicon oil bath at 95° C. until complete dissolution. 10.6 gr of PVP was added to the PVA solution and reacted for 1.5 hours. The solution was cooled to 78° C., then 16.6 gr of urea-hydrogen peroxide (UHP) and 6 gr of thymol was added and short mixed until homogeneous white suspension obtained. The hydrogel suspension was molded into a 250 ml beaker. The obtained PVA/PVP/HP/thymol mold was then placed in a freezer (approximately −18° C.) overnight and then thawed (freeze/thaw cycle). The hydrogel capsule was pulled out from the beaker and was wrapped with perforated PE sheet. The hydrogel capsule was then stored in 4° C.

### Preparation of Thymol-Based Oil in Water Emulsions

### Thymol Emulsion

Required amount of PVP (and/or PVP and PVA) was dissolved in distilled water. Appropriate amount of thymol was suspended into the PVP (or PVP/PVA) solution by applying heat (75° C.) until milky white emulsion obtained and then let cool down to room temperature.

### Silver Hydrogen Peroxide

Predetermined volume of hydrogen peroxide solution was mixed with silver nitrate solution with the desired final ratio.

### Silver Hydrogen Peroxide and Thymol Emulsion

Silver hydrogen peroxide solution and thymol emulsion were mixed with homogenizer.

### Phytotoxicity Tests

The emulsions were sprayed on different varieties of seedlings. Phytotoxicity monitoring was done by visual inspection during the 48-72 h from the test beginning. The treated seedlings demonstrated wide range of phytotoxicity effects, started from none or minor signs to more dominant damages.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. An article comprising a coated substrate, wherein the coated substrate comprises a polymeric substrate, wherein at least one surface of said substrate is in contact with a coating layer comprising a hydrogel, wherein:
said hydrogel comprises PVA and PVP at a w/w PVA:PVP ratio between about 15:1 and about 1:1;
a water content of said hydrogel is between 50 and 90% w/w
and wherein said coating layer is characterized by a dry thickness between 1 μm and 500 μm.

2. The article of claim 1, wherein said substrate comprises at least partially oxidized surface, comprising a plurality of surface modifications selected from hydroxy, carbonyl, and carboxy, including any combination thereof.

3. (canceled)

4. The article of claim 1, wherein the coating layer further comprises an active agent, optionally wherein the active agent has an antimicrobial activity, a plant stimulating activity, a pesticidal activity or any combination thereof.

5. The article of claim 4, wherein the active agent comprises hydrogen peroxide (HP), or a precursor thereof, halogen gas precursor, an essential oil, a fertilizer, a pesticide, and a plant hormone, or any combination thereof, and wherein a weight portion of said active agent within said coating is between 0.01 and 20%.

6. (canceled)

7. The article of claim 4, characterized by a sustained release of the active agent from said article, wherein said sustained comprises a substantial release of said active agent within a time period between 1 day and 12 months.

8. The article of claim 1, wherein the substrate is stably adhered to the coating layer; and wherein said coating layer is characterized by a visible light transmission (VLT) between 60% and 99%.

9. (canceled)

10. The article of claim 1, wherein said polymeric substrate comprises a polymer selected from polyolefin (e.g. polyethylene (PE), polypropylene (PP), high-density polyethylene (HDPE), low-density polyethylene (LDPE), very low-density polyethylene (VLDPE)), polyethylene terephthalate (PET), PET derivatives, polymethylmethacrylate(PMMA), polystyrene (PS), polyvinyl alcohol (PVA), polycarbonate (PC), silicon rubber polyester, polyvinyl chloride (PVC), polyacetal, cellulose, cellulose derivatives, poly(2-hydroxyethyl methacrylate) (pHEMA), nylon, including any copolymer and any combination thereof.

11. The article of 4, wherein the substrate comprises a polyolefin, the w/w PVA:PVP ratio is between about 4:1 and about 2:1, and wherein the active agent comprises (i) the essential oil and (ii) HP, or a precursor thereof; wherein a w/w concentration of the active agent is between about 1 and 10%; and wherein a w/w ratio between (i) and (ii) is between 2:1 and 1:2.

12. (canceled)

13. The article of claim 4, wherein said article is an antimicrobial article; optionally wherein said article is selected from: a film, a ribbon, a package, a wound dressing, a container, and a plant article.

14. (canceled)

15. An article comprising a hydrogel comprising PVA and PVP at a w/w PVA:PVP ratio between about 15:1 and about 1:1; wherein:
a water content of said hydrogel is between 50 and 90% w/w;
said hydrogel is cross-linked via a crosslinking agent.
said hydrogel further comprises an active agent incorporated within the hydrogel;
and a w/w concentration of the active agent within the hydrogel is between 0.1 and 20%

16. The article of claim 15, wherein said article is manufactured by molding or by casting an aqueous composition comprising the hydrogel; and wherein a w/w concentration of the cross-linking agent within the hydrogel is between 0.5 and 20%.

17. (canceled)

18. The article of claim 15, wherein the cross-linking agent is (i) a polyfunctional cross-linking agent comprising a plurality of moieties capable of reacting with PVA, optionally the cross-linking agent comprises a dialdehyde, or a polyaldehyde; or (ii) is a complexation agent capable of forming complexation interaction with PVA.

19. The article of claim 15, wherein said article is (i) in a form of a film, wherein the film is characterized by a dry thickness between 1 um and 500 um; or (ii) in a form of a plant article comprising a plurality of walls, wherein at least one wall comprises the hydrogel and is characterized by a dry thickness between 0.1 and 20 mm.

20. (canceled)

21. The article of claim 15, characterized by a sustained release of the active agent from said article into an ambient, wherein said sustained release comprises a substantial release of said active agent within a time period between 1 day and 12 months. wherein said article is light transparent, optionally characterized by a visible light transmission (VLT) between 60% and 99%; and wherein said article is an agricultural article, a pharmaceutical article, an antimicrobial article, or an injectable pharmaceutical composition, comprising an effective amount of the active agent.

22. (canceled)

23. (canceled)

24. A composition comprising a plurality of beads, each of the plurality of beads comprises a hydrogel comprising PVA and PVP at a w/w PVA:PVP ratio between about 15:1 and about 1:1; wherein:
a water content of said hydrogel is between 10 and 90% w/w;
said hydrogel further comprises an active agent incorporated within the hydrogel.
a w/w concentration of the active agent within the hydrogel is between 0.5 and 20%; said hydrogel is cross-linked via a cross-linking agent.

25. The composition of claim 24, wherein a w/w concentration of the cross-linking agent within the hydrogel is between 0.5 and 10%; wherein the cross-linking agent is selected from boric acid and a dialdehyde, or any combination thereof, and wherein a w/w PVA:PVP ratio between about 3:1 and about 1:1; and wherein each of the plurality of beads further comprises between 0.1 and 5% w/w of alginate.

26. (canceled)

27. (canceled)

28. The composition of claim 24, wherein the active agent comprises HP, or a precursor thereof, halogen gas precursor, an essential oil, a fertilizer, a pesticide, and a plant hormone, a viable organism, or any combination thereof; optionally wherein an average cross-section of the plurality of beads is between 1 and 50 mm.

29. (canceled)

30. The composition of claim 24, wherein said composition is an agricultural composition comprising an agriculturally effective amount of the active agent, and optionally comprises an agriculturally acceptable carrier, an additive, or both.

31. The composition of claim 24, being a pesticidal composition configured for (i) reducing or eradicating a pest, (ii) for preventing or reducing a plant disease associated with the pest, or both (i) and (ii), when applied to a plant part, or to an area under cultivation.

32. The composition of claim 30, wherein said agricultural composition is configured for (i) controlling plant growth or ripening; (ii) delivering an agricultural agent to the plant or both (i) and (ii), when applied to the plant to the plant part, or to an area under cultivation-reducing comprises at least 10% reduction.

33.-53. (canceled)
